(19) United States
(12) Patent Application Publication
(10) Pub. No.: US 2025/0381180 A1
(43) Pub. Date: Dec. 18, 2025

(54) METHODS OF TREATING OR PREVENTING OVERACTIVE BLADDER SYNDROME

(71) Applicant: Purdue Pharma L.P., Stamford, CT (US)

(72) Inventors: Garth WHITESIDE, Yardley, PA (US); Stephen HARRIS, Weston, CT (US)

(73) Assignee: Purdue Pharma L.P., Stamford, CT (US)

(21) Appl. No.: 18/878,591

(22) PCT Filed: Jun. 23, 2023
(86) PCT No.: PCT/US2023/026149

§ 371 Date: Dec. 23, 2024

Related U.S. Application Data
(60) Provisional application No. 63/366,975, filed on Jun. 24, 2022.

(51) Int. Cl. A61K 31/498; A61K 9/00; A61K 31/137; A61K 31/216; A61K 31/4025; A61K 31/46; A61K 31/4725; A61P 13/10

(52) U.S. Cl. CPC A61K 31/498; A61K 9/0053; A61K 31/137; A61K 31/216; A61K 31/4025; A61K 31/46; A61K 31/4725; A61P 13/10

(57) ABSTRACT

The disclosure provides a method of treating or preventing overactive bladder syndrome (OBS) in a human subject (with normal renal function, mild renal impairment, or mild to moderate renal impairment) in need of such treatment, comprising administering to the human subject a therapeutically effective amount of a compound of the formula (I) or a pharmaceutically acceptable salt thereof. In one embodiment, the method comprises administering the compound of formula (IA).

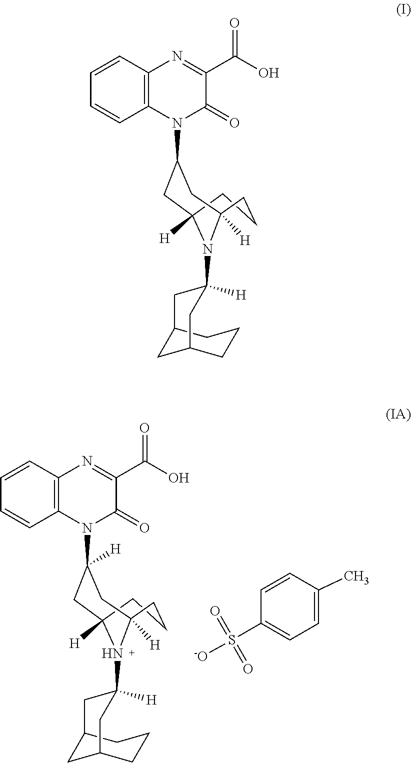

### BACKGROUND

Overactive bladder syndrome can occur when the muscles of the bladder begin to involuntarily contract, even if the volume of urine in the bladder is low. These muscle contractions create an urgent need to urinate. Patients suffering with overactive bladder syndrome (OBS) generally urinate more frequently than healthy individuals and often wake up one or more times at night to urinate (nocturia). Many patients with OBS experience urinary incontinence, which is defined as unintentional loss of urine.

OBS results from the abnormal and involuntary contractions of the detrusor muscle of the bladder, which is embedded by muscarinic receptors. As the bladder fills with urine, it begins to stretch. Such stretching, which is sensed by afferent neurons, results in the desire to urinate. Nerves in the muscular wall of the bladder release the neurotransmitter acetylcholine, which binds to the muscarinic receptors on the muscle walls of the bladder and causes the cells to contract, hence additionally increasing the desire to urinate. Stimulation of these muscarinic receptors by acetylcholine causes bladder contractions that lead to urination. Normally, the detrusor muscle remains at rest as the bladder fills with urine. However, in patients with OBS, the bladder contracts during the filling phase.

Common pharmacological treatments for OBS are antimuscarinic agents (i.e., muscarinic receptor antagonists) that inhibit the stimulation of muscle by the neurotransmitter acetylcholine. Several antimuscarinic agents have been approved for use in treating OBS, including oxybutynin, tolterodine, trospium, solifenacin and darifenacin. By blocking the effect of acetylcholine on the muscle cells, antimuscarinic agents slows the build-up of pressure in the bladder, reduces the sensation to urinate, and prevents uncontrolled urination.

Administration of antimuscarinic agents effects the efferent pathway associated with muscle contraction that results in urination. Less success has been realized with treatments affecting the sensory (afferent) pathways associated with urination. Afferent, nerve fibers travel from the lower urinary tract to the spinal cord through the pelvic, hypogastric and pudendal nerves. Afferent nerves in the lower urinary tract are made of two fibers (A-δ and C-fibers) that are associated with sensations of pressure and respond to the stretch of the bladder when the bladder fills with urine. The afferent neurons express different types of receptors and ion channels, including transient receptor potential channels, purinergic, muscarinic, endothelin, neurotrophic factor, and estrogen receptors. These receptors have various functions, many which involve enhancing or diminishing neuronal excitability. One role of these afferent fibers is to transmit information concerning bladder pressure to the central nervous system, hence initiating the efferent pathway.

There is a need for effective treatments of lower urinary tract disorders using small-molecule therapeutics that can be orally administered.

### SUMMARY

The present disclosure provides a method of treating or preventing overactive bladder syndrome (OBS) through administration of a small-molecule agonist of the nociception opioid peptide receptor, also referred to as the ORL-1 receptor.

In one aspect, the disclosure provides a method of treating or preventing OBS in a human subject identified in need of such treatment, comprising administering to the human subject a therapeutically effective amount of a compound of formula (I):

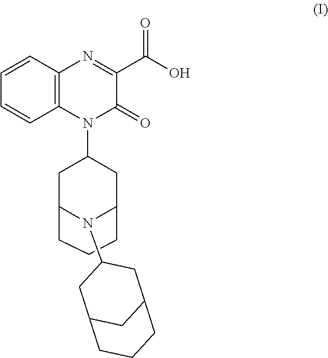 [SMILES: O=C(O)c1nc2ccccc2n(C2CC3CCCC(C2)N3C2CC3CCCC(C3)C2)c1=O]

or a pharmaceutically acceptable salt thereof. The compound of formula (I) includes all its stereoisomers (including enantiomers) and polymorphic forms thereof.

In some embodiments, the disclosure provides a method of treating or preventing overactive bladder syndrome (OBS) in a human subject identified in need of such treatment, comprising administering to the subject a therapeutically effective amount of a compound having the formula (I′):

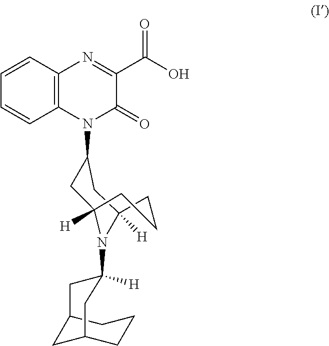

or a pharmaceutically acceptable salt thereof. As well appreciated by an ordinarily skilled person in the art, the compound of formula (I′) is a stereoisomer of the compound of formula (I).

In certain embodiments, the compound of formula (I) or formula (I′) is administered as a tosylate salt. For instance, in certain embodiments, the disclosure provides a method of treating or preventing overactive bladder syndrome (OBS) in a human subject in need of such a treatment, comprising administering to the subject a therapeutically effective amount of a compound of the formula (IA):

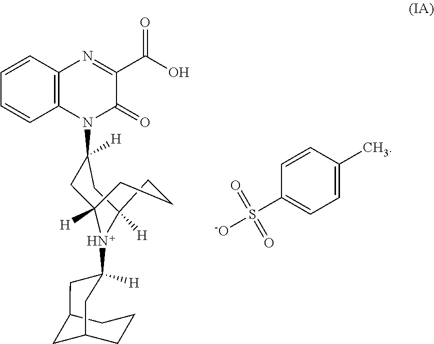

In certain embodiments, the disclosure provides methods of treating or preventing one or more symptoms associated with OBS in a human subject identified in need of such treatment by administering a compound of formula (I) or (I′), or a pharmaceutically acceptable salt thereof, wherein the one or more symptoms are selected from the group of urinary incontinence, urgency and enhanced urinary frequency. In some embodiments, the method comprises administering a compound of formula (IA).

In certain embodiments, the compound of formula (I) or (I′), or a pharmaceutically acceptable salt thereof, is administered orally. As referred to hereinafter, the compounds of the disclosure include the compounds of formulae (I) and (I′) (including all stereoisomers), pharmaceutically acceptable salts (e.g., the compound of formula (IA)), polymorphic forms, solvates, or hydrates thereof. A suitable effective dosage amount of a compound of the disclosure as a single dose for oral administration is from about 0.001 mg to about 30 mg, about 0.10 mg to about 10 mg, from about 0.50 mg to about 8 mg, from about 1 mg to about 6 mg, or from about 1 mg to about 3 mg. In some such embodiments, the compound to be administered is a compound of the formula (IA).

In particular embodiments, the compound of the disclosure, is administered orally. In certain embodiments, the compound to be administered is a compound of the formula (IA). It is understood that oral administration of the compound of the disclosure (e.g., the compound of formula (I), (I′), or (IA)) results in high concentrations of the compound in the bladder.

A particular advantage of the disclosed methods is that they are capable of simultaneously treating patients who suffer from both sleep disorders and lower urinary tract complications (e.g., OBS). Therefore, in one aspect, the disclosure provides methods of treating or preventing OBS in a human subject who also suffers from a sleep disorder, comprising administering to the subject a therapeutically effective amount of a compound of the disclosure. In certain embodiments, the compound is the compound of the formula (IA). In some embodiments, the human subject suffers from insomnia. In one embodiment, the human subject suffers from insomnia associated with alcohol cessation. In some embodiments, the compound of the disclosure is administered at nighttime.

### BRIEF DESCRIPTION OF FIGURES

FIG. 1A shows the basal value of rhythmic bladder contraction (RBC) frequency in anesthetized female rats following administration of vehicle, the compound of formula (IA) (marked as Cpd (IA)), or tolterodine; FIG. 1B shows the basal value of RBC amplitude in anesthetized female rats following administration of vehicle, the compound of formula (IA), or tolterodine. Results are expressed in mean±s.e.m.^{N.S.} p>0.05, one-way ANOVA.

FIG. 1C shows a diagram of a study designed to evaluate the effects of intravenous administration of the compound of formula (IA) on cystometric parameters in an isovolumetric model in anesthetized female rats.

FIG. 2A shows the effect of vehicle (1 mL/kg, i.v.) on RBC frequency in anesthetized female rats; FIG. 2B shows the effect of the compound of formula (1A) (3 mg/kg, i.v.) on RBC frequency in anesthetized female rats; FIG. 2C shows the effect of the compound of tolterodine (1 mg/kg, i.v.) on RBC frequency in anesthetized female rats. Results are expressed in mean±s.e.m., * p<0.05, ** p<0.01 and **** p<0.0001, versus basal, paired Student t-test or Wilcoxon test.

FIG. 3 shows the effect of vehicle (1 mL/kg, i.v.), the compound of formula (1A) (3 mg/kg, i.v.), and tolterodine (1 mg/kg, i.v.) on RBC frequency expressed as % of variation from basal levels in anesthetized female rats. Results are expressed in mean±s.e.m.^{N.S.} p>0.05 and ⁺⁺⁺⁺ p<0.0001, versus vehicle, Kruskal Wallis followed by Dunn's post-test.

FIG. 4A shows the effect of vehicle (1 mL/kg, i.v.) on RBC amplitude in anesthetized female rats; FIG. 4B shows the effect of the compound of formula (1A) (3 mg/kg, i.v.) on RBC amplitude in anesthetized female rats; FIG. 4C shows the effect of the compound of tolterodine (1 mg/kg, i.v.) on RBC amplitude in anesthetized female rats. Results are expressed in mean±s.e.m., * p<0.05 and ** p<0.01 versus basal, paired Student t-test or Wilcoxon test.

FIG. 5 shows the effect of vehicle (1 mL/kg, i.v.), the compound of formula (IA) (3 mg/kg, i.v.), and tolterodine (1 mg/kg, i.v.) on RBC amplitude expressed as % of variation from basal levels in anesthetized female rats. RBC amplitudes are expressed as % variation from basal values. Results are expressed in mean±s.e.m.^{N.S.} p>0.05 and p<0.001, versus vehicle, one-way ANOVA followed by Dunnett's post-test.

FIG. 6 shows the effect of vehicle (1 mL/kg, i.v.), the compound of formula (1A) (3 mg/kg, i.v.), and tolterodine (1 mg/kg, i.v.) on time of inhibition in anesthetized female rats. Results are expressed in mean±s.e.m.^{N.S.} p>0.05 and ** p<0.01, versus vehicle, one-way ANOVA followed by Dunnett's post-test.

FIG. 7 shows a recording of analyzed cystometric parameters for vehicle (1 mL/kg, i.v.) (7A), the compound of the formula (IA) (3 mg/kg, i.v.) (7B); and tolterodine (1 mg/kg, i.v.) (7C).

FIG. 8 demonstrates a typical recording of analyzed cystometric parameters.

FIG. 9 is a scheme of SCI study protocol design.

FIG. 10 presents the cystometric parameters measured in the SCI study.

FIG. 11 demonstrates effects of SCI on cystometric parameters: BC (11A), ThP (11B), AM (11C), NVC frequency (11D), and NVC amplitude (11E) and bladder weight (11F). Results are expressed as mean±s.e.m.^{ns}p>0.05, ^{§§} p<0.01, ^{§§§} p<0.001, ^{§§§§} p<0.0001, unpaired Student 1-test.

FIG. 12 demonstrates basal values of cystometric parameters: BC (12A), ThP (12B), AM (12C), NVC frequency (12D), and NVC amplitude (12E); and bladder weight (12F) in all SCI experimental rats. Results are expressed as mean±s.e.m.^{NS}p>0.05, one way ANOVA or Kruskal-Wallis test.

FIG. 13 shows effects of vehicle (5 mL/kg, i.g., n=12, once) in sham rats (13A), vehicle (5 mL/kg, i.g., n=11, once) in SCI rats (13B), mirabegron (10 mg/kg, i.g., n=13, once) in SCI rats (13C), and Cpd (IA) (30 mg/kg, i.g., n=12, once) in SCI rats (13D), versus Basal. Results are expressed as mean±s.e.m.^{NS} p>0.05, ** p<0.01 *** p<0.001 one-way ANOVA with repeated measures followed by Dunnett's post-test or Friedman test followed by Dunn's post-test vs Basal.

FIG. 14 shows effects of mirabegron (10 mg/kg, i.g.) and Cpd (IA) (30 mg/kg, i.g.) versus vehicle (5 mL/kg, i.g.) on BC expressed in mL (14A) and in % variation from basal value (14B). Results are expressed as mean±s.e.m.^{NS} p>0.05, * p<0.05, Kruskal Wallis test followed by Dunn's post-test, vs Vehicle.

FIG. 15 shows effects of vehicle (5 mL/kg, i.g.) in sham rats (15A) and in SCI rats (15B), mirabegron (10 mg/kg, i.g.) in SCI rats (15C) and Cpd (IA) (30 mg/kg, i.g.) in SCI rats (15D) on THP versus basal value. Results are expressed as mean±s.e.m.^{NS} p>0.05, * p<0.05, one way ANOVA with repeated measures or Friedman test followed by Dunn's post-test, vs Basal.

FIG. 16 shows effects of mirabegron (10 mg/kg, i.g.) and Cpd (IA) (30 mg/kg, i.g.) versus vehicle (5 mL/kg, i.g.) on ThP expressed in mmHg (16A) and in % variation from basal value (16B). Results are expressed as mean±s.e.m.^{NS} p>0.05 Kruskal Wallis test, vs Vehicle.

FIG. 17 shows effects of vehicle (5 mL/kg, i.g.) in sham rats (17A) and in SCI rats (17B), mirabegron (10 mg/kg, i.g.) in SCI rats (17C) and Cpd (IA) (30 mg/kg, i.g.) in SCI rats (17D) on AM versus basal value. Results are expressed as mean±s.e.m.^{NS} p>0.05, * p<0.05, one way ANOVA with repeated measures or Friedman test vs Basal.

FIG. 18 shows effects of mirabegron (10 mg/kg, i.g.) and Cpd (IA) (30 mg/kg, i.g.) versus vehicle (5 mL/kg, i.g.) on AM expressed in mmHg (18A) and in % variation from basal value (18B). Results are expressed as mean±s.e.m.^{NS} p>0.05 Kruskal Wallis test followed by Dunn's post-test, vs Vehicle.

FIG. 19 shows effects of vehicle (5 mL/kg, i.g.) in sham rats (19A) and in SCI rats (19B), mirabegron (10 mg/kg, i.g.) in SCI rats (19C) and Cpd (IA) (30 mg/kg, i.g.) in SCI rats (19D) on NVC frequency versus basal value. Results are expressed as mean #s.e.m.^{NS} p>0.05, *p<0.05, ** p<0.01, one-way ANOVA with repeated measures followed by Dunnett's post-test or Friedman test followed by Dunn's post-test vs Basal.

FIG. 20 shows effects of mirabegron (10 mg/kg, i.g.) and Cpd (IA) (30 mg/kg, i.g.) versus vehicle (5 mL/kg, i.g.) on NVC frequency expressed in NVC/min (20A) and NS p>0.05, in % variation from basal value (20B). Results are expressed as mean±s.e.m. NS one-way ANOVA, vs Vehicle.

FIG. 21 shows effects of vehicle (5 mL/kg, i.g.) in sham rats (21A) and in SCI rats (21B), mirabegron (10 mg/kg, i.g.) in SCI rats (21C) and Cpd (IA) (30 mg/kg, i.g.) in SCI rats (21D) on NVC amplitude versus basal value. Results are expressed as mean±s.e.m.^{NS} p>0.05, *p<0.05, *** p<0.01, one-way ANOVA with repeated measures followed by Dunnett's post-test or Friedman test followed by Dunn's post-test, vs Basal

FIG. 22 shows effects of mirabegron (10 mg/kg, i.g.) and Cpd (IA) (30 mg/kg, i.g.) versus vehicle (5 mL/kg, i.g.) on NVC amplitude expressed in mmHg (22A) and in % variation from basal value (22B). Results are expressed as mean±s.e.m.^{NS} p>0.05, one-way ANOVA or Kruskal Wallis test, vs Vehicle.

FIG. 23A-F show basal values of cystometric parameters and bladder weights in all BOO experimental rats. Results are expressed as mean #s.e.m.^{NS} p>0.05, unpaired Student 1-test.

FIG. 24 shows effects of vehicle (1 mL/kg, i.v.) (24A) and Cpd (IA) (3 mg/kg, i.v.) (24B) on BC in BOO rats for the 0-90 min entire period versus Basal. Results are expressed as mean±s.e.m.^{ns} p>0.05, paired Student/test or Wilcoxon test vs Basal.

FIG. 25 shows effects of Cpd (IA) (3 mg/kg, i.v.) versus vehicle (1 mL/kg, i.v.) on BC expressed in mL (25A) and in % variation from basal value (25B) for the 0-90 min entire period. Results are expressed as mean±s.e.m.^{ns} p>0.05 and ** p<0.01, unpaired Student/test or Mann-Whitney test, vs Vehicle.

FIG. 26 shows effects of vehicle (1 mL/kg, i.v.) (26A) and Cpd (IA) (3 mg/kg, i.v.) (26B) on BC in BOO rats for the 60-90 min entire period vs Basal. Results are expressed as mean±s.e.m.^{ns} p>0.05, paired Student/test or Wilcoxon test vs Basal.

FIG. 27 shows effects of Cpd (IA) (3 mg/kg, i.v.) versus vehicle (1 mL/kg, i.v.) on BC expressed in mL (27A) and in % variation from basal value (27B) for the 60-90 min entire period. Results are expressed as mean±s.e.m.^{NS}s p>0.05, unpaired Student t test or Mann-Whitney test vs Vehicle.

FIG. 28 shows effects of vehicle (1 mL/kg, i.v.) (28A) and Cpd (IA) (3 mg/kg, i.v.) (28B) on ThP in BOO rats for the 0-90 min entire period vs Basal. Results are expressed as mean±s.e.m.^{ns} p>0.05, paired Student/test vs Basal.

FIG. 29 shows effects of Cpd (IA) (3 mg/kg, i.v.) versus vehicle (1 mL/kg, i.v.) on ThP expressed in mmHg (29A) and in % variation from basal value (29B) for the 0-90 min entire period. Results are expressed as mean±s.e.m.^{NS}$ p>0.05, unpaired Student t test vs Vehicle.

FIG. 30 shows effects of vehicle (1 mL/kg, i.v.) (30A) and Cpd (IA) (3 mg/kg, i.v.) (30B) on ThP in BOO rats for the 60-90 min entire period vs Basal. Results are expressed as mean #s.e.m.^{ns}p>0.05, paired Student/test vs Basal.

FIG. 31 shows effects of Cpd (IA) (3 mg/kg, i.v.) versus vehicle (1 mL/kg, i.v.) on ThP expressed in mmHg (31A) and in % variation from basal value (31B) for the 60-90 min entire period. ^{ns}p>0.05 and ⁺p<0.05, unpaired Student/test vs Vehicle.

FIG. 32 shows effects of vehicle (1 mL/kg, i.v.) (32A) and Cpd (IA) (3 mg/kg, i.v.) (32B) on AM in BOO rats for the 0-90 min entire period vs Basal. Results are expressed as mean±s.e.m.^{ns} p>0.05, paired Student/test or Wilcoxon test vs Basal.

FIG. 33 shows effects of Cpd (IA) (3 mg/kg, i.v.) versus vehicle (1 mL/kg, i.v.) on AM expressed in mmHg (33A) and in % variation from basal value (33B) for the 0-90 min entire period. Results are expressed as mean±s.e.m.^{ns} p>0.05, paired Student/test or Mann-Whitney test vs Basal.

FIG. 34 shows effects of vehicle (1 mL/kg, i.v.) (34A) and Cpd (IA) (3 mg/kg, i.v.) (34B) on AM expressed in mmHg in BOO rats for the 60-90 min entire period vs Basal. Results are expressed as mean±s.e.m.^{ns}p>0.05, paired Student/test or Wilcoxon test vs Basal.

FIG. 35 shows effects of Cpd (IA) (3 mg/kg, i.v.) versus vehicle (1 mL/kg, i.v.) on AM expressed in mmHg (35A) and in % variation from basal value (35B) for the 60-90 min entire period. Results are expressed as mean±s.e.m.^{ns} p>0.05, paired Student/test or Mann-Whitney test vs Basal.

FIG. 36 shows effects of vehicle (1 mL/kg, i.v.) (36A) and Cpd (IA) (3 mg/kg, i.v.) (36B) on NVC frequency in BOO rats for the 0-90 min entire period vs Basal. Results are expressed as mean #s.e.m.^{ns}p>0.05, paired Student/test vs Basal.

FIG. 37 shows effects of Cpd (IA) (3 mg/kg, i.v.) versus vehicle (1 mL/kg, i.v.) on NVC frequency expressed in NVC/min (37A) and in % variation from basal value (37B) for the 0-90 min entire period. Results are expressed as mean±s.e.m.^{ns}p>0.05, paired Student/test or Mann-Whitney test vs Basal.

FIG. 38 shows effects of vehicle (1 mL/kg, i.v.) (38A) and Cpd (IA) (3 mg/kg, i.v.) (38B) on NVC frequency in BOO rats for the 60-90 min entire period vs Basal. Results are expressed as mean±s.e.m.^{ns}p>0.05, paired Student/test or Wilcoxon test vs Basal

FIG. 39 shows effects of Cpd (IA) (3 mg/kg, i.v.) versus vehicle (1 mL/kg, i.v.) on NVC frequency expressed in NVC/min (39A) and in % variation from basal value (39B) for the 60-90 min entire period. Results are expressed as mean±s.e.m.^{ns} p>0.05, one-way ANOVA followed by Dunnett's post-test vs vehicle.

FIG. 40 shows effects of vehicle (1 mL/kg, i.v.) (40A) and Cpd (IA) (3 mg/kg, i.v.) (40B) on NVC amplitude in BOO rats for the 0-90 min entire period vs Basal. Results are expressed as mean #s.e.m.^{ns}p>0.05, paired Student/test or Wilcoxon test vs Basal.

FIG. 41 shows effects of Cpd (IA) (3 mg/kg, i.v.) versus vehicle (1 mL/kg, i.v.) on NVC amplitude expressed in mmHg (41A) and in % variation from basal value (41B) for the 0-90 min entire period. Results are expressed as mean±s.e.m.^{ns} p>0.05, paired Student/test or Mann-Whitney test vs Basal.

FIG. 42 shows effects of vehicle (1 mL/kg, i.v.) (42A) and Cpd (IA) (3 mg/kg, i.v.) (42B) on NVC amplitude in BOO rats for the 60-90 min entire period vs Basal. Results are expressed as mean±s.e.m.^{ns}p>0.05, paired Student/test or Wilcoxon test vs Basal.

FIG. 43 shows effects of Cpd (IA) (3 mg/kg, i.v.) versus vehicle (1 mL/kg, i.v.) on NVC amplitude expressed in mmHg (43A) and in % variation from basal value (43B) for the 60-90 min entire period. Results are expressed as mean±s.e.m.^{ns}p>0.05, unpaired Student/test or Mann-Whitney test vs Vehicle.

FIG. 44 shows a diagram of a phase 1, nonrandomized, open-label, parallel-group, single-dose study design.

FIG. 45A shows mean plasma concentrations versus time profiles on a linear scale for subjects with normal renal function (n=10) and subjects with mild renal impairment (n=10).

FIG. 45B shows mean plasma concentrations versus time profiles on a semi-logarithmic scale on a linear scale for subjects with normal renal function (n=10) and subjects with mild renal impairment (n=10).

FIG. 46A shows plasma concentrations versus time profiles on a linear scale for individual subjects with mild renal impairment (n=10).FIG. 46B shows plasma concentrations versus time profiles on a semi-logarithmic scale for individual subjects with mild renal impairment (n=10).

FIG. 47A shows plasma concentrations versus time profiles on a linear scale for individual subjects with normal renal function (n=10).

FIG. 47B shows plasma concentrations versus time profiles on a semi-logarithmic scale for individual subjects with function (n=10).

### DETAILED DESCRIPTION

The disclosure provides methods of treating or preventing pathological conditions associated with overstimulation of afferent nerves associated with the lower urinary tract. Such pathological conditions include overactive bladder system (OBS), urinary urgency, increased voiding frequency, nocturia and urinary incontinence. Specifically, the disclosure provides methods of treating or preventing pathological conditions associated with overstimulation of afferent nerves through administration of an agonist of the nociceptin opioid peptide receptor, also referred to as the ORL-1 receptor.

Identification of the ORL-1 receptor as distinct from the three long-known major classes of opioid receptors in the central nervous system-mu, kappa, and delta-resulted from experimentation on these opioid receptor classes. The ORL-1 receptor was identified and classified as an opioid receptor based only on amino acid sequence homology, as the ORL-1 receptor did not exhibit overlapping pharmacology with the classic mu opioid receptor. It was initially demonstrated that non-selective ligands having a high affinity for mu, kappa, and delta receptors had low affinity for the ORL-1 receptor. This characteristic, along with the fact that an endogenous ligand had not yet been discovered, led to the term “orphan receptor.” See, e.g., Henderson et al., “The orphan opioid receptor and its endogenous ligand-nociceptin/orphanin FQ,” Trends Pharmacol. Sci. 18 (8): 293-300 (1997). Subsequent research led to the isolation and structure of the endogenous ligand of the ORL-1 receptor (i.e., nociceptin; also known as orphanin FQ or OFQ), a seventeen amino acid peptide structurally similar to members of the opioid peptide family. For a general discussion of ORL-1 receptors, see Calo′ et al., “Pharmacology of nociceptin and its receptor: a novel therapeutic target,” Br. J. Pharmacol. 129:1261-1283 (2000).

The endogenous ligand for the ORL-1 receptor is a 17-amino acid peptide referred to as nociceptin. Through interaction with ORL-1, nociceptin possesses inhibitory activity of the micturition reflux in various animal models. Direct administration of nociception and peptide analogs of nociception into the bladder has been studied as a means of alleviating urinary incontinence, presumably through diminishing afferent signaling (Lazzeri et al., 2003 Urology; Lazzeri et al., 2006 J. Urology; & Del Popolo et al., 2011).

The inventors have discovered unexpectedly that the pathological conditions associated with overstimulation of afferent nerves associated with the lower urinary tract can be ameliorated or treated through the administration of a therapeutically effective amount of a compound of formula (I):

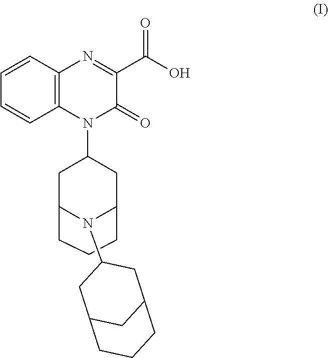 [SMILES: O=C(O)c1nc2ccccc2n(C2CC3CCCC(C2)N3C2CC3CCCC(C3)C2)c1=O]

or a pharmaceutically acceptable salt thereof. The compound of formula (I) includes all stereoisomers (including enantiomers) and polymorphic forms thereof.

In certain embodiments, the compound of formula (I) is a single stereoisomer, that is, the compound of formula (I′) having the structure depicted below:

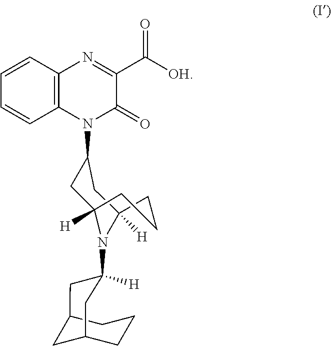

The compounds of formula (I) and formula (I′), or pharmaceutically acceptable salts thereof, can be prepared as described in U.S. Pat. No. 8,476,221, which is hereby incorporated by reference.

Accordingly, in one aspect, the disclosure provides a method of treating or preventing overactive bladder syndrome (OBS) in a human subject in need of such treatment, comprising administering to the subject a therapeutically effective amount of a compound of the disclosure, e.g., the compound of the formula (I) or (I′), or a pharmaceutically acceptable salt (e.g., the compound of formula (IA)) thereof. In certain embodiments, administration of the compound of the disclosure also improves (e.g., alleviates the severity of) symptoms associated with OBS, including, but not limited to, urinary urgency, increased voiding frequency, nocturia and urinary incontinence.

In one embodiment, administration of the compound of the disclosure increases the pressure threshold for micturition by from about 10% to about 99.5%. In another embodiment, administration of the compound of the disclosure increases the pressure threshold for micturition by from about 20% to about 90%. In another embodiment, administration of the compound of the disclosure increases the pressure threshold for micturition by from about 30% to about 80%.

In another aspect, the disclosure provides method of reducing occurrences of nocturia in a human subject in need of such treatment, comprising administering to the subject a therapeutically effective amount of a compound of the disclosure. In some embodiments, nighttime administration refers to administration of the compound prior to bedtime. In some embodiments, the compound of the disclosure can be administered anytime from about three hours prior to bedtime until right before bedtime of the human subject. In one embodiment, instances of nighttime urination are reduced from 2 or more times each night to less than 2 times following nightly administration of a compound of the disclosure. For instance, the number of times the subject urinates at night can be reduced from 2 or more times to 0 or 1 time following nightly administration of a compound of the disclosure.

In certain embodiments, the method of this disclosure comprises administering to the human subject a therapeutically effective amount of a compound of the disclosure once every two nights. In other embodiments, the method comprises administering to the human subject a therapeutically effective amount of a compound of the disclosure once every three nights. In another embodiment, the method comprises administering to the human subject a therapeutically effective amount of a compound of the disclosure once every week. In other embodiments, the method comprises administering to the human subject a therapeutically effective amount of a compound of the disclosure once every three nights. In still another embodiment, the method comprises administering to the human subject a therapeutically effective amount of a compound of the disclosure twice every week.

In another aspect, the disclosure provides method of treating or preventing urinary incontinence in a human subject identified in need of such treatment, comprising administering to the subject a therapeutically effective amount of a compound of the disclosure.

In another aspect, the disclosure provides methods of treating or preventing OBS in a human subject by administering a therapeutically effective amount of a compound of the disclosure, wherein the compound works through inhibiting contraction of the detrusor muscle in the bladder of the human subject. In some embodiments, contraction of the detrusor muscle in the bladder can be delayed (in time interval) by at least 20% following administration of a compound of the disclosure. In other embodiments, contraction of the detrusor muscle in the bladder can be delayed by at least 30% following administration of a compound of the disclosure. In other embodiments, contraction of the detrusor muscle in the bladder can be delayed by at least 50% following administration of a compound of the disclosure. In other embodiments, contraction of the detrusor muscle in the bladder can be delayed by at least 70% following administration of a compound of the disclosure. In other embodiments, contraction of the detrusor muscle in the bladder can be delayed by at least 80% following administration of a compound of the disclosure. In other embodiments, contraction of the detrusor muscle in the bladder can be delayed by at least 90% following administration of a compound of the disclosure. In other embodiments, contraction of the detrusor muscle in the bladder can be delayed by from about 20% to about 90%, from about 30% to about 85%, from about 40% to about 80%, or from about 50% to about 70% following administration of a compound of the disclosure

In another aspect, the disclosure provides methods of treating or preventing OBS in a human subject by administering a compound of the disclosure (e.g., the compound of formula (I) or (I′), or a pharmaceutically acceptable salt thereof), wherein the compound works by decreasing the frequency of contractions of the detrusor muscle in the bladder of the subject. In some embodiments, the frequency of contraction of the detrusor muscle in the bladder decreases by at least 20% following administration of a compound of the disclosure. In other embodiments, the frequency of contraction of the detrusor muscle in the bladder decreases by at least 30% following the administration of a compound of the disclosure. In other embodiments, the frequency of contraction of the detrusor muscle in the bladder decreases by at least 50% following the administration of a compound of the disclosure. In other embodiments, the frequency of contraction of the detrusor muscle in the bladder decreases by at least 70% following administration of a compound of the disclosure. In other embodiments, the frequency of contraction of the detrusor muscle in the bladder decreases by at least 80% following the administration of a compound of this disclosure. In other embodiments, the frequency of contraction of the detrusor muscle in the bladder decreases by at least 90% following the administration of a compound of the disclosure. In certain embodiments, the frequency of contraction of the detrusor muscle in the bladder decreases by from about 20% to about 90%, from about 30% to about 85% m from about 40% to about 80%, or from about 50% to about 70% following the administration of a compound of the disclosure.

The terms “treatment of,” “treating,” and related terms as used herein include the amelioration, alleviation, reduction, slowing, or cessation of a Condition or a symptom thereof by administration of an effective amount of a compound of the disclosure, e.g., the compound of formula (I) or (I′), or a pharmaceutically acceptable salt (e.g., the compound of formula (IA)) thereof. In some embodiments, treating includes inhibiting (for example, decreasing the overall frequency of) episodes of a Condition (e.g., OBS) or a symptom (e.g., urinary incontinence or nocturia) thereof, or reducing the severity of a Condition or a symptom thereof.

The terms “prevention of,” “preventing,” and related terms as used herein include the avoidance of the onset of a Condition or a symptom thereof by administration of an effective amount of a compound of formula (I), or a pharmaceutically acceptable salt thereof.

The term “effective amount,” when used in connection with methods of the disclosure, refers to an amount of a compound, when administered to an animal (e.g., a human subject), that provides a partial or full therapeutic effect that is desired by a skilled artisan (e.g., a physician) through such an administration.

The term “therapeutically effective amount,” when used in connection with methods of the disclosure, refers to an amount of the compound administered to an animal (e.g., a human subject) that provides a desired therapeutic effect.

Without wishing to be bound by any theory, it is believed that the compound of the disclosure exerts its beneficial effects through modulation of the ORL-1 receptor expressed on the afferent nerve fibers/endings in the lower urinary tract. The terms “modulate,” “modulating,” and related terms as used herein with respect to the ORL-1 receptor mean the mediation of a pharmacodynamic response (e.g., OBS) in an animal from (i) inhibiting or activating the receptor, or (ii) directly or indirectly affecting the normal regulation of the receptor activity. Compounds that modulate the receptor activity include agonists, partial agonists, biased agonists, antagonists, mixed agonists/antagonists, mixed partial agonists/antagonists and compounds which directly or indirectly affect regulation of the receptor activity. The compound of formula (I) and (I′), and pharmaceutically acceptable salts (e.g., the compound of formula (IA)) thereof, are partial agonists. As used herein, a compound that binds to a receptor and is only partly effective as an agonist, as compared to another agonist including the native ligand, is defined as a “partial agonist.” It is believed that the partial agonists of the disclosure can achieve the desired therapeutic effects (e.g., treatment of OBS), without any or with fewer concurrent side effects often associated with the administration of full agonists.

As used herein, the term “eGFR” refers to an estimated glomerular filtration rate (eGFR) that is calculated with the formula shown below:
eGFR=142 ×min (standardized Scr/k,1)α×max (standardized Scr/k, 1)-1200×0.9938Age×1.012 [if female] where k is 0.7 for females and 0.9 for males, α is −0.241 for females and −0.302 for males, min indicates the minimum of Scr/k or 1, and max indicates the maximum of Scr/k or 1.

As used herein, a human subject with “no renal insufficiency” or with “normal renal function” means that the human subject has an eGFR of 90 mL/min or more.

As used herein, a human subject with “mild renal impairment” means that the human subject has an eGFR of about 60 mL/min to about 89 mL/min.

As used herein, a human subject with “mild to moderate renal impairment” means that the human subject has an eGFR of about 45 mL/min to about 59 mL/min.

The compound of the disclosure can be administered as a component of a composition that comprises a pharmaceutically acceptable carrier or excipient. Routes of administration include, but are not limited to, oral, intravesical. Intradermal, intramuscular, intraperitoneal, parenteral, intravenous, subcutaneous, intranasal, epidural, transmucosal, buccal, gingival, sublingual, intraocular, intracerebral, intravaginal, transdermal (e.g., via a patch), rectal, by inhalation, or topical. In another embodiment, routes of administration include, but are not limited to, intravenous, intravesical, oral, or by inhalation. In another embodiment, the route of administration is oral. In another embodiment, the route of administration is intravesical. In another embodiment, the route of administration is intravenous. In another embodiment, the route of administration is by inhalation.

In yet another embodiment, a compound of the disclosure can be delivered in a controlled-release system or sustained-release system. As well understood in the art (e.g., pharmaceutical industry), controlled- or sustained-release pharmaceutical compositions can improve drug therapy over that achieved by their non-controlled or non-sustained-release counterparts (e.g., immediate-release formulations). In one embodiment, a controlled- or sustained-release composition comprises a therapeutically effective amount of a compound of formula (I) or (I′), or a pharmaceutically acceptable salt thereof, to treat or prevent OBS or a symptom thereof for an extended amount of time. Advantages of controlled- or sustained-release compositions include extended activity of the drug, reduced dosage frequency, and increased compliance.

Administration of a compound of the disclosure can be by controlled-release or sustained-release means or by delivery devices that are known to those in the art. Examples include, but are not limited to, those described in U.S. Pat. Nos. 3,845,770, 3,916,899, 3,536,809, 3,598,123, 4,008,719, 5,674,533, 5,059,595, 5,591,767, 5,120,548, 5,073,543, 5,639,476, 5,354,556, and 5,733,566, each of which is incorporated herein by reference. Numerous other controlled-release or sustained-release delivery devices that are known to those in the art (see, e.g., Goodson, “Dental Applications,” in Medical Applications of Controlled Release, Vol. 2, Applications and Evaluation, Langer and Wise, eds., CRC Press, Chapter 6, pp. 115-138 (1984), hereafter “Goodson”). Other controlled- or sustained-release systems discussed in the review by Langer, Science 249:1527-1533 (1990) can be used. In one embodiment, a pump can be used (Langer, Science 249:1527-1533 (1990); Sefton, “Implantable Pumps,” in (′R C Crit. Rev. Biomed. Eng. 14 (3): 201-240 (1987); Buchwald et al., “Long-term, Continuous Intravenous Heparin Administration by an Implantable Infusion Pump in Ambulatory Patients with Recurrent Venous Thrombosis,” Surgery 88:507-516 (1980); and Saudek et al., “A Preliminary Trial of the Programmable Implantable Medication System for Insulin Delivery,” New Engl. J. Med. 321:574-579 (1989)). In another embodiment, polymeric materials can be used (see Goodson; Smolen et al., “Drug Product Design and Performance,” Controlled Drug Bioavailability Vol. 1, John Wiley and Sons, New York (1984); Langer et al., “Chemical and Physical Structure of Polymers as Carriers for Controlled Release of Bioactive Agents: A Review,” J. Macromol. Sci. Rev. Macromol. Chem. C23 (1): 61-126 (1983); Levy et al., “Inhibition of Calcification of Bioprosthetic Heart Valves by Local Controlled-Release Diphosphonate,” Science 228:190-192 (1985); During et al., “Controlled Release of Dopamine from a Polymeric Brain Implant: In Vivo Characterization,” Ann. Neurol. 25:351-356 (1989); and Howard et al., “Intracerebral drug delivery in rats with lesion-induced memory deficits,” J. Neurosurg. 71:105-112 (1989)).

Suitable dosage forms can be used to provide controlled- or sustained-release of one or more active ingredients using, for example, hydroxypropyl methyl cellulose, ethyl cellulose, other polymer matrices, gels, permeable membranes, osmotic systems, multilayer coatings, microparticles, multiparticulates, liposomes, microspheres, or a combination thereof to provide the desired release profile in varying proportions. Suitable controlled- or sustained-release formulations known to those in the art, including those described herein, can be readily selected for use with the active ingredients of the disclosure. The disclosure thus encompasses single unit dosage forms suitable for oral administration such as, but not limited to, tablets, capsules, gelcaps, and caplets that are adapted for controlled- or sustained-release.

The compositions can optionally, but preferably, further comprise a suitable amount of a pharmaceutically acceptable excipient to provide the form for proper administration to the animal. Such a pharmaceutical excipient can be a diluent, suspending agent, solubilizer, binder, disintegrant, preservative, coloring agent, lubricant, and the like. The pharmaceutical excipient can be a liquid, such as water or an oil, including those of petroleum, animal, vegetable, or synthetic origin, such as peanut oil, soybean oil, mineral oil, sesame oil, and the like. The pharmaceutical excipient can be saline, gum acacia, gelatin, starch paste, talc, keratin, colloidal silica, urea, and the like. In addition, auxiliary, stabilizing, thickening, lubricating, and coloring agents can be used. In one embodiment, the pharmaceutically acceptable excipient is sterile when administered to an animal. Water is a particularly useful excipient when a compound of formula (I), or a pharmaceutically acceptable salt thereof, is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The compositions, if desired, can also contain minor amounts of wetting or emulsifying agents, or pH buffering agents. Specific examples of pharmaceutically acceptable carriers and excipients that can be used to formulate oral dosage forms are described in the Handbook of Pharmaceutical Excipients, (Amer. Pharmaceutical Ass′n, Washington, DC, 1986), incorporated herein by reference. Other examples of suitable pharmaceutical excipients are described by Radebough et al., “Preformulation,” pp. 1447-1676 in Remington's Pharmaceutical Sciences Vol. 2 (Gennaro, ed., 19th Ed., Mack Publishing, Easton, PA, 1995), incorporated herein by reference.

In one embodiment, the compound of the disclosure is formulated in accordance with routine procedures as a composition adapted for oral administration to human beings. A compound of the disclosure to be orally delivered can be in the form of tablets, capsules, gelcaps, caplets, lozenges, aqueous or oily solutions, suspensions, granules, microparticles, multiparticulates, powders, emulsions, syrups, or elixirs, for example. When a compound of formula (I) or (I′), or a pharmaceutically acceptable salt thereof, is incorporated into oral tablets, such tablets can be compressed, tablet triturates, enteric-coated, sugar-coated, film-coated, multiply compressed, or multiply layered. Techniques and compositions for making solid oral dosage forms are described in Pharmaceutical Dosage Forms: Tablets (Lieberman et al., eds., 2^{nd} Ed., Marcel Dekker, Inc., 1989 and 1990). Techniques and compositions for making tablets (compressed and molded), capsules (hard and soft gelatin) and pills are also described by King, “Tablets, Capsules, and Pills,” pp. 1553-1593 in Remington's Pharmaceutical Sciences (Osol, ed., 16th Ed., Mack Publishing, Easton, PA, 1980).

Liquid oral dosage forms include aqueous and non-aqueous solutions, emulsions, suspensions, and solutions and/or suspensions reconstituted from non-effervescent granules, optionally containing one or more suitable solvents, preservatives, emulsifying agents, suspending agents, diluents, sweeteners, coloring agents, flavoring agents, and the like. Techniques and composition for making liquid oral dosage forms are described in Pharmaceutical Dosage Forms: Disperse Systems (Lieberman et al., eds., 2^{nd} Ed., Marcel Dekker, Inc., 1996 and 1998).

An oral pharmaceutical composition containing a compound of the disclosure (e.g., the compound of formula (I) or (I′), or a pharmaceutically acceptable salt thereof) can contain one or more excipients, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

The compositions can take the form of solutions, suspensions, emulsions, tablets such as an orally disintegrating tablet (ODT) or a sublingual tablet, pills, pellets, capsules, capsules containing liquids, powders, sustained-release formulations, suppositories, emulsions, aerosols, sprays, suspensions, microparticles, multiparticulates, rapidly dissolving films or other forms for oral or mucosal administration, or any other form suitable for use. In one embodiment, the composition is in the form of an ODT (see, e.g., U.S. Pat. Nos. 7,749,533 and 9,241,910). In another embodiment, the composition is in the form of a sublingual tablet (see, e.g., U.S. Pat. Nos. 6,572,891 and 9,308,175). In another embodiment, the composition is in the form of a capsule (see, e.g., U.S. Pat. No. 5,698,155). In another embodiment, the composition is in a form suitable for buccal administration, e.g., as a tablet, lozenge, gel, patch, or film, formulated in a conventional manner (see, e.g., Pather et al., “Current status and the future of buccal drug delivery systems,” Expert Opin. Drug Deliv. 5 (5): 531-542 (2008)). In another embodiment, the composition is in a form suitable for gingival administration, e.g., as a polymeric film comprising polyvinyl alcohol, chitosan, polycarbophil, hydroxypropylcellulose, or Eudragit S-100, as disclosed by Padula et al., “In Vitro Evaluation of Mucoadhesive Films for Gingival Administration of Lidocaine,” AAPS PharmSciTech 14 (4): 1279-1283 (2013). In another embodiment, the composition is in a form suitable for intraocular administration.

In one embodiment, the compound of the disclosure is formulated for parenteral administration. When a compound of the disclosure is to be injected parenterally, it can be, e.g., in the form of an isotonic sterile solution.

When a compound of the disclosure is administered parenterally, the formulation for parenteral administration can be in the form of a suspension, solution, emulsion in an oily or aqueous vehicle. Such formulations can further comprise pharmaceutically necessary additives such as one or more stabilizing agents, suspending agents, dispersing agents, buffers, and the like. A compound of the disclosure can also be in the form of a powder for reconstitution as an injectable formulation.

In another embodiment, the compounds of the disclosure can be formulated for intravenous administration. In certain embodiments, compositions for intravenous administration comprise sterile isotonic aqueous buffer. Where necessary, the compositions can also include a solubilizing agent. An intravenous composition containing a compound of the disclosure can optionally include a local anesthetic such as benzocaine or prilocaine to lessen pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where a compound of the disclosure is to be administered by infusion, it can be dispensed, for example, with an infusion bottle containing sterile pharmaceutical grade water or saline. Where a compound of the disclosure is administered by injection, an ampule of sterile water for injection or saline can be provided so that the ingredients can be mixed prior to administration.

The compound of the disclosure, e.g., the compound of formula (I) or (I′), is primarily excreted from the urine largely unchanged (See Example 2 below). Therefore, the compound of formula (I) or (I′) mostly concentrates in the bladder following administration. For instance, when administered orally, the compound of formula (I) or (I′) excreted in the urine ranges from about 30% to about 95%, depending on the dose administered. Additionally, the concentration of the compound of formula (I) or (I′) in the urine remains high for at least 24 hours following oral administration. In particular embodiments, the concentration of the compound of formula (I) or (I′) after 12 hours following oral administrations is greater than 100 nM. In other embodiments, the concentration of the compound of formula (I) or (I′) at the 12-hour mark following oral administrations is greater than 500 nM. In other embodiments, the concentration of the compound of formula (I) or (I′) at the 12-hour mark following oral administrations is greater than 1,000 nM. In other embodiments, the concentration of the compound of formula (I) or (I′) at the 12-hour mark following oral administrations is greater than 5,000 nM. In other embodiments, the concentration of the compound of formula (I) or (I′) 12 hours following oral administrations is greater than 10,000 nM. In particular embodiments, the concentration of the compound of formula (I) or (I′) 12 hours after oral administration can range from about 100 nM to about 30,000 nM. In other embodiments, the concentration of the compound of formula (I) or (I′) 12 hours after oral administration can range from about 500 nM to about 15,000 nM. In other embodiments, the concentration of the compound of formula (I) or (I′) 12 hours after oral administration can range from about 1,000 nM to about 10,000 nM.

In some embodiments, the compound of formula (I) or (I′) is administered in the form of a pharmaceutically acceptable salt. The term “pharmaceutically acceptable salt,” as used herein, is any pharmaceutically acceptable salt (including both inorganic salts and organic salts) that can be prepared from a compound of formula (I). Illustrative salts include, but are not limited, to sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. In one embodiment, the pharmaceutically acceptable salt is a hydrochloride salt, a sulfate salt, a sodium salt, a potassium salt, a benzene sulfonic acid salt, a para-toluenesulfonic acid salt, or a fumaric acid salt. In another embodiment, the pharmaceutically acceptable salt is a hydrochloride salt or a sulfate salt. In another embodiment, the pharmaceutically acceptable salt is a hydrochloride salt. In another embodiment, the pharmaceutically acceptable salt is a sulfate salt. In another embodiment, the pharmaceutically acceptable salt is a sodium salt. In another embodiment, the pharmaceutically acceptable salt is a potassium salt. In another embodiment, the pharmaceutically acceptable salt is a fumaric acid salt. In another embodiment, the pharmaceutically acceptable salt is a p-toluenesulfonate, that is, a para-toluenesulfonic acid salt (also known as, “tosylate salt”). In another embodiment, the pharmaceutically acceptable salt is a choline salt.

In another embodiment, the pharmaceutically acceptable para-toluenesulfonic acid salt contains one equivalent of a compound of formula (I) or (I′) and about 1.0 equivalent of para-toluenesulfonic acid, e.g., from about 0.8 to about 1.2 equivalents of para-toluenesulfonic acid in one embodiment, from about 0.9 to about 1.1 equivalents of para-toluenesulfonic acid in another embodiment, from about 0.93 to about 1.07 equivalents of para-toluenesulfonic acid in another embodiment, from about 0.95 to about 1.05 equivalents of para-toluenesulfonic acid in another embodiment, from about 0.98 to about 1.02 equivalents of para-toluenesulfonic acid in another embodiment, or from about 0.99 to about 1.01 equivalents of para-toluenesulfonic acid in another embodiment. In another embodiment, the pharmaceutically acceptable para-toluenesulfonic acid salt contains about one equivalent of a compound of formula (I′) and about one equivalent of para-toluenesulfonic acid, i.e., is a mono-tosylate salt. In another embodiment, the pharmaceutically acceptable para-toluenesulfonic acid salt contains one equivalent (relative to the amount of p-toluenesulfonic acid) of a compound of formula (I). In another embodiment, the pharmaceutically acceptable para-toluenesulfonic acid salt contains one equivalent of a compound of formula (I′). The mono-tosylate salt of the compound of formula (I′), i.e., the compound of formula (IA), is as follows:

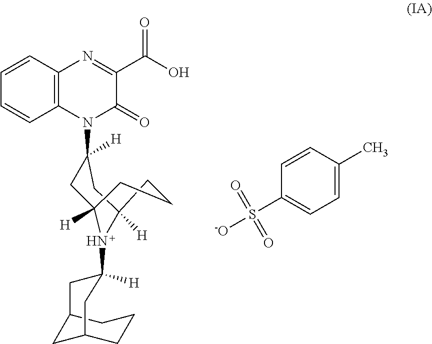

The methods of the disclosure provided herein also encompass the use of any solvate of the compound of formula (I) or (I′), or a pharmaceutically acceptable salt thereof. “Solvate” are generally known in the art, and are considered herein to be a combination, physical association and/or solvation of a compound of formula (I) or (I′), or a pharmaceutically acceptable salt thereof. This physical association can involve varying degrees of ionic and covalent bonding, including hydrogen bonding. When the solvate is of the stoichiometric type, there is a fixed ratio of the solvent molecule to the compound of formula (I) or (I′), or a pharmaceutically acceptable salt thereof. A compound of formula (I) or (I′), or a pharmaceutically acceptable salt thereof can be present as a solvated form with a pharmaceutically acceptable solvent, such as water (e.g., a hydrate), methanol, ethanol, and the like.

The methods of the disclosure provided herein also encompass the use of any crystalline form (or polymorphic form) of the compounds of formula (I) or (I′), or a pharmaceutically acceptable salt thereof. The term “crystalline” and related terms used herein, when used to describe a substance, component or product, means that the substance, component or product is substantially crystalline as determined by X-ray diffraction, microscopy, polarized microscopy, or other known analytical procedure known to those skilled in the art. The term “polymorph,” as used herein, refers to crystalline forms of a compound having different unit cell structures in crystals, originating from a variety of molecular conformations and molecular packing. Polymorphs of a single compound can have one or more different chemical, physical, mechanical, electrical, thermodynamic, and/or biological properties from each other. Differences in physical properties exhibited by polymorphs can affect pharmaceutical parameters such as storage stability, compressibility, density (important in composition and product manufacturing), dissolution rates (an important factor in determining bio-availability), solubility, melting point, chemical stability, physical stability, powder flowability, water sorption, compaction, and particle morphology. Differences in stability can result from changes in chemical reactivity (e.g., differential oxidation, such that a dosage form discolors more rapidly when comprised of one polymorph than when comprised of another polymorph), or mechanical changes (e.g., crystal changes on storage as a kinetically favored polymorph converts to a thermodynamically more stable polymorph) or both (e.g., one polymorph is more hygroscopic than the other.

In certain embodiments, the compound of formula (IA) has the crystalline form referred to as Form A, Form B, Form C, Form D, or Form E, as described in WO 2020/157691, the contents of which are incorporated herein by reference. In some embodiments, the compound of formula (IA) is of crystalline Form A. In other embodiments, the compound of formula (IA) is of crystalline Form B. In other embodiments, the compound of formula (IA) is of crystalline Form C. In other embodiments, the compound of formula (IA) is of crystalline Form D. In other embodiments, the compound of formula (IA) is of crystalline Form E.

The amount by weight of the administered “dose,” “dosage,” and related terms as used herein refers to the free acid and free base form of a compound of formula (I) or (I′), i.e., the no-salt form. For example, a 10.0 mg dose means that 10.0 mg of the no-salt form of the compound of formula (I) or (I′) is actually administered. However, by way of example, a 10.0 mg dose of, e.g., the monohydrochloride or the 1:1 by moles hydrochloric acid salt of the compound of formula (I) or (I′) means that 10.84 mg of said compound is actually administered, which 10.84 mg provides 10.00 mg of the no-salt form of the compound of formula (I) or (I′) (0.0229 mmoles) and 0.84 mg of hydrochloric acid (0.0229 mmoles). Likewise, a 10.00 mg dose of, e.g., the mono-tosylate salt (1:1 by moles para-toluenesulfonic acid salt) of the compound of formula (IA), means that 13.93 mg of said compound is actually administered, which 13.93 mg provides 10.00 mg of the no-salt form of the compound of formula (I) or (I′) (0.0229 mmoles) and 3.93 mg of para-toluenesulfonic acid (0.0229 mmoles).

In terms of a method for treating or preventing a Condition or a symptom in a human subject, suitable effective dosage amounts of a compound of the disclosure are from about 0.0002 mg/kg to about 10 mg/kg of body weight of the human subject per day in one embodiment, from about 0.00025 mg/kg/day to about 5 mg/kg/day in another embodiment, from about 1.5 mg/kg/day to about 3 mg/kg/day in another embodiment, from about 0.2 mg/kg/day to about 2 mg/kg/day in another embodiment, from about 2.5 mg/kg/day to about 10.0 mg/kg/day in another embodiment, and from about 3.0 mg/kg/day to about 5.0 mg/kg/day in another embodiment. In another embodiment, the effective dosage amount is about 10 mg/kg/day or less. In certain embodiments, suitable effective dosage amounts of the compound of formula (I) or (I′), or a pharmaceutically acceptable salt thereof, are from about 0.0002 mg/kg/day to about 10 mg/kg/day, from about 0.001 mg/kg/day to about 10 mg/kg/day, from about 0.002 mg/kg/day to about 10 mg/kg/day, from about 0.003 mg/kg/day to about 10 mg/kg/day, from about 0.0005 mg/kg/day to about 5.0 mg/kg/day, from about 0.001 mg/kg/day to about 2.5 mg/kg/day, from about 0.002 mg/kg/day to about 2.0 mg/kg/day, or from about 0.002 mg/kg/day to about 1.0 mg/kg/day. In another embodiment, the effective dosage amount is about 1.0 mg/kg/day or less. In certain other embodiments, suitable effective dosage amounts of the compound of formula (I) or (I′), or a pharmaceutically acceptable salt thereof, are from about 0.001 mg/kg/day to about 1.0 mg/kg/day, from about 0.002 mg/kg/day to about 0.8 mg/kg/day, from about 0.0025 mg/kg/day to about 0.5 mg/kg/day, from about 0.003 mg/kg/day to about 0.15 mg/kg/day, from about 0.006 mg/kg/day to about 0.12 mg/kg/day, or from about 0.010 mg/kg/day to about 0.10 mg/kg/day. It is to be understood that for these dosage amounts, the term “day” means a 24-hour cycle beginning at the time of administration of a compound of the disclosure, e.g., the compound of formula (I) or (I′), or a pharmaceutically acceptable salt thereof.

In embodiments where the compound of formula (I) or (I′), or a pharmaceutically acceptable salt (e.g., the compound of formula (IA)) thereof, is administered orally, a suitable effective dosage amount of the compound as a single dose is from about 0.001 mg to about 300 mg, from about 0.005 mg to about 250 mg, from about 0.01 mg to about 200 mg, from about 0.05 mg to about 150 mg, from about 0.075 mg to about 50 mg, or from about 0.10 mg to about 10 mg. In one embodiment, the compound of the disclosure is administered as a single dose in a non-controlled or non-sustained release formulation (e.g., an immediate-release formulation). In another embodiment, the effective dosage amount of the compound of the disclosure is administered as multiple doses in non-controlled or non-sustained release formulations.

In certain embodiments, about 0.05 mg, about 0.06 mg, about 0.07 mg, about 0.08 mg, about 0.09 mg, about 0.100 mg, about 0.120 mg, about 0.125 mg, about 0.150 mg, about 0.175 mg, about 0.200 mg, about 0.225 mg, about 0.250 mg, about 0.275 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1.00 mg, about 1.25 mg, about 1.50 mg, about 1.75 mg, about 2.00 mg, about 2.25 mg, about 2.50 mg, about 2.75 mg, about 3.00 mg, about 3.25 mg, about 3.50 mg, about 3.75 mg, about 4.0 mg, about 4.5 mg, about 5.0 mg, about 5.5 mg, about 6.0 mg, about 6.5 mg, about 7.0 mg, about 7.5 mg, about 8.0 mg, about 9.0 mg, or about 10 mg of the compound of formula (I) or (I′), or a pharmaceutically acceptable salt (e.g., the compound of formula (IA)) thereof, is administered orally to a human subject in need thereof. In some embodiments, about 1 mg of the compound of formula (I) or (I′), or an equivalent amount of a pharmaceutically acceptable salt thereof, is administered orally to a human subject in need thereof. In some embodiments, about 1.5 mg of the compound of formula (I) or (I′), or an equivalent amount of a pharmaceutically acceptable salt thereof, is administered orally to a human subject in need thereof. As known to those in the art, for a human animal, a single daily dose (in mg) can be converted to a mg/kg/day dosage amount by dividing the mg dose by 60 kg, the art-recognized average mass of a human animal. For example, a single daily human dose of 12 mg is so-converted to a dosage amount of about 0.20 mg/kg/day.

In certain embodiments, a controlled-release composition comprising a therapeutically effective amount of a compound of the disclosure is administered as a single dose or in multiple doses. The controlled-release composition may contain up to 100 times of the dosage amount of the compound of formula (I) or (I′), or a pharmaceutically acceptable salt thereof (e.g., the compound of formula (IA)), that is used for a non-controlled or non-sustained-release formulation.

In some embodiments, the compound of formula (I) or (I′), or a pharmaceutically acceptable salt thereof, can be administered once daily. In some such embodiments, the compound of formula (I) or (I′) is administered nightly (e.g., before bedtime). As shown in Example 2, daily administration of the compound of formula (I) or (I′) results in urine concentration that is orders of magnitude greater than the in vitro activity of the Compound of formula (IA) as measured by Ki and EC₅₀.

In addition to its beneficial effects of treating or preventing conditions associated with overactive bladder, the compound of the disclosure, when administered at sufficient dose levels, is also capable of inducing drowsiness and treating sleep disorders. See U.S. Publication No. 2020/0345726, which is hereby incorporated by reference. Patients suffering from symptoms associated with overactive bladder often suffer from poor sleep quality, insomnia, and/or nocturia. In some embodiments, the patients suffering from sleep disorders are females 50 years of age or older. In other embodiments, the patients suffering from sleep disorders are males 50 years of age or older. Nightly administration of the compound of formula (I) or (I′), or a pharmaceutically acceptable salt thereof, improves both sleep quality and symptoms associated with overactive bladder. In certain embodiments, the compound of formula (I) or (I′), or a pharmaceutically acceptable salt thereof, is administered by the patient each night prior to sleep. For instance, the compound of formula (I) or (I′), or a pharmaceutically acceptable salt thereof, can be administered from about 1 minute to about 3 hours prior to sleep. In some embodiments, the compound of formula (I) or (I′), or a pharmaceutically acceptable salt thereof, can be administered from about 5 minutes to about 60 minutes prior to sleep. In other embodiments, the compound of formula (I) or (I′), or a pharmaceutically acceptable salt thereof, can be administered from about 10 minutes to about 30 minutes prior to sleep.

In one embodiment, an effective dose or dosage amount of a compound of the disclosure (e.g., the compound of formula (I) or (I′), or a pharmaceutically acceptable salt thereof) is administered about 60 minutes before a human's median habitual bedtime. In another embodiment, an effective dose or dosage amount is administered about 45 minutes before a human's median habitual bedtime. In another embodiment, an effective dose or dosage amount is administered about 30 minutes before a human's median habitual bedtime. In another embodiment, an effective dose or dosage amount is administered about 20 minutes before a human's median habitual bedtime. In another embodiment, an effective dose or dosage amount is administered about 20 minutes or less before a human's median habitual bedtime. In another embodiment, an effective dose or dosage amount is administered about 15 minutes before a human's median habitual bedtime. In another embodiment, an effective dose or dosage amount is administered about 15 minutes or less before a human's median habitual bedtime. In another embodiment, an effective dose or dosage amount is administered about 10 minutes before a human's median habitual bedtime. In another embodiment, an effective dose or dosage amount is administered about 10 minutes or less before a human's median habitual bedtime. In another embodiment, an effective dose or dosage amount is administered about 5 minutes before a human's median habitual bedtime.

In certain embodiments, the compound of formula (I) or (I′), or a pharmaceutically salt thereof, can be administered multiple times during the day. For instance, the compound of formula (I) or (I′), or a pharmaceutically salt thereof, can be administered twice daily or three times daily. In embodiments where the compound is administered multiple times daily, each dose may be administered in the same amount or in different amounts. In some embodiments, the dose of the compound of formula (I) or (I′), or pharmaceutically acceptable salt thereof, is administered at a higher dose than the other doses provided earlier in the day. In some embodiments, the compound of formula (I) or (I′), or pharmaceutically acceptable salt thereof, is administered twice daily, approximately every 12 hours. In other embodiments, the compound of formula (I) or (I′), or pharmaceutically acceptable salt thereof, is administered three times daily, approximately every 8 hours.

In certain embodiments, the compound of the disclosure is administered twice daily, wherein the second dose (i.e., the second therapeutically effective amount) is administered prior to bedtime, as set forth above. In some such embodiments, the second dose is administered at a higher amount than the first dose. For instance, the second dose can be administered at an amount of about 1.5-fold, 2-fold, 3-fold-, 5-fold, 10-fold, 20-fold, 50-fold. 100-fold or 1000-fold higher than the first dose. In some embodiments, the second dose can be administered in an amount from about 1.5-fold to about 10-fold higher than the first dose. In other embodiments, the second dose can be administered in an amount from about 1.5-fold to about 100-fold higher than the first dose. In other embodiments, the second dose can be administered in an amount from about 1.5-fold to about 1000-fold higher than the first dose. In other embodiments, the second dose can be administered in an amount from about 3-fold to about 100-fold higher than the first dose. In other embodiments, the second dose can be administered in an amount from about 3-fold to about 1000-fold higher than the first dose. In other embodiments, the second dose can be administered in an amount from about 5-fold to about 100-fold higher than the first dose. In other embodiments, the second dose can be administered in an amount from about 5-fold to about 1000-fold higher than the first dose. Such dosing schedules ensure that the first dose is effective at treating or preventing OBS and symptoms associated with OBS without causing residual drowsiness, whereas the second dose is effective at treating or preventing OBS and symptoms associated with OBS and causing drowsiness or inducing sleep in a human subject. In certain embodiments, both the first dose and the second dose are administered through (same or different) non-controlled or non-sustained release formulations. In other embodiments, the first dose is administered through a controlled or non-sustained release formulation, and the second dose is administered through a non-controlled or non-sustained release formulation.

In another embodiment, a composition comprising a compound of the disclosure is useful as a medicament in the treatment of human subjects suffering from both OBS and a particular sleep disorder. Such sleep disorders include, but are not limited to, an insomnia condition, a hypersomnia condition, a circadian rhythm sleep-wake disorder, an alcohol-induced sleep disorder, or any combination thereof. Other sleep disorders include an alcohol-induced sleep disorder (e.g., insomnia-type alcohol-induced sleep disorder, daytime sleepiness type alcohol-induced sleep disorder, parasomnia type alcohol-induced sleep disorder, and mixed type alcohol-induced sleep disorder); insomnia in alcohol use disorder; sleep disturbances associated with alcohol cessation (e.g., insomnia associated with alcohol cessation); or any combination thereof. In one embodiment, the human subject suffers from both OBS and insomnia associated with alcohol cessation.

The methods for treating or preventing a lower urinary tract disorder (e.g., OBS) in a human subject (e.g., a patient) in need thereof can further comprise co-administering to the human subject a compound of the disclosure (e.g., the compound of formula (I) or (I′), or a pharmaceutically acceptable salt thereof), and a second therapeutic agent. In one embodiment, the second therapeutic agent is administered in an effective amount for achieving the desired therapeutic effects according to the methods of the disclosure. In one embodiment, the second therapeutic agent is an antimuscarinic agent. In some such embodiments, the antimuscarinic agent is oxybutynin. In other such embodiments, the antimuscarinic agent is tolterodine. In other such embodiments, the antimuscarinic agent is trospium. In separate embodiments, the antimuscarinic agent is solifenacin. In certain embodiments, the antimuscarinic agent is darifenacin. In other such embodiments, the antimuscarinic agent is flavoxate. Also disclosed herein is a method of treating overactive bladder syndrome in a human subject in need thereof, comprising administering to the human subject an effective amount of a compound of formula (I):

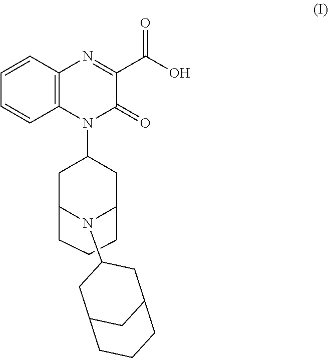 [SMILES: O=C(O)c1nc2ccccc2n(C2CC3CCCC(C2)N3C2CC3CCCC(C3)C2)c1=O]

or a pharmaceutically acceptable salt thereof, wherein the human subject has a mild or mild to moderate renal impairment. In some embodiments, the human subject with mild renal impairment can have an estimated glomerular filtration rate (eGFR) of about 60 mL/min to about 89 mL/min. In some embodiments, the human subject with mild to moderate renal impairment can have an eGFR of about 45 mL/min to about 59 mL/min.

In some embodiments, administering the compound of formula (I) to the subject can result in at least a mean AUC, Cₘₐₓ, Tₘₐₓ, T_{1/2}, or CL/F that is not statistically different from a corresponding mean AUC, Cₘₐₓ, Tₘₐₓ, T_{1/2}, or CL/F in a similarly situated human subject with no renal insufficiency (or with normal renal function). As used herein, a human subject is considered to be “similarly situated” when the human subject satisfy the same inclusion/exclusion criteria as the other human subject who is being treated with the methods of the disclosure, and also match other relevant characteristics in clinical studies, such as, gender, age, body weight, and body mass index.

As used herein, “AUC” refers to an area under the concentration-time curve. As used herein, “Cₘₐₓ” refers to a maximum observed plasma concentration. As used herein, “T max” refers to a time to reach the maximum observed plasma concentration. As used herein, “T_{1/2}” refers to an apparent terminal half-life. As used herein, “CL/F” refers to an apparent total body clearance.

In some embodiments, administering the compound of formula (I) to the subject can result in at least a mean AUC that is not statistically different from a corresponding mean AUC in a similarly situated human subject with no renal insufficiency (or with normal renal function). In some embodiments, administering the compound of formula (I) to the subject can result in at least a mean Cₘₐₓ that is not statistically different from a corresponding mean Cₘₐₓ in a similarly situated human subject with no renal insufficiency. In some embodiments, administering the compound of formula (I) to the subject can result in at least a mean Tₘₐₓ, that is not statistically different from a corresponding mean Tₘₐₓ in a similarly situated human subject with no renal insufficiency. In some embodiments, administering the compound of formula (I) to the subject can result in at least a mean T_{1/2} that is not statistically different from a corresponding mean T_{1/2} in a similarly situated human subject with no renal insufficiency. In some embodiments, administering the compound of formula (I) to the subject can result in at least a mean CL/F that is not statistically different from a corresponding mean CL/F in a similarly situated human subject with no renal insufficiency.

In some embodiments, administering the compound of formula (I) to the human subject can result in at least a mean Ae, Fe, or CL_{R} that is not statistically different from a corresponding mean Ae, Fe, or CL_{R} in a similarly situated human subject with no renal insufficiency. As used herein, “Ae” refers to an amount of drug excreted unchanged in urine after study drug dosing. As used herein, “Fe” refers to the fraction of the study drug excreted in urine, calculated as, e.g., (Ae₀₋₉₆/Dose). As used herein, “CL_{R}” refers to the estimated renal clearance of the study drug for the total collection interval (e.g., 0-96 hours), calculated as, e.g., (Ae₀₋₉₆/AUC_{inf}).

In some embodiments, administering the compound of formula (I) to the human subject can result in at least a mean Ae that is not statistically different from a corresponding mean Ae in a similarly situated human subject with no renal insufficiency. In some embodiments, administering the compound of formula (I) to the human subject can result in at least a mean Fe that is not statistically different from a corresponding mean Fe in a similarly situated human subject with no renal insufficiency. In some embodiments, administering the compound of formula (I) to the human subject can result in at least a mean CL_{R} that is not statistically different from a corresponding mean CL_{R} in a similarly situated human subject with no renal insufficiency.

In some embodiments, the compound of formula (I) is a compound of formula (I′):

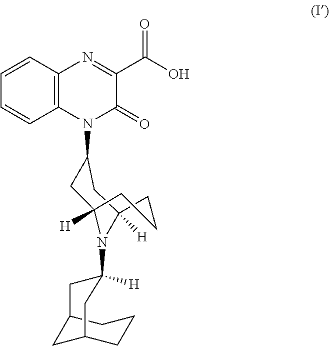

or a pharmaceutically acceptable salt thereof. In some embodiments, the method can comprise administering a pharmaceutically acceptable salt of the compound of formula (I), wherein the salt is selected from the group consisting of sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, and p-toluenesulfonate salts.

In some embodiments, the method can comprise administering a p-toluenesulfonic acid salt (i.e., a p-toluenesulfonate salt), a sulfate salt, a phosphoric acid salt, or a hydrochloride salt of the compound of formula (I). In some embodiments, a p-toluenesulfonic acid salt of the compound of formula (I) can be administered.

In some embodiments, the compound of formula (I) or the pharmaceutically acceptable salt thereof is a compound of formula (IA):

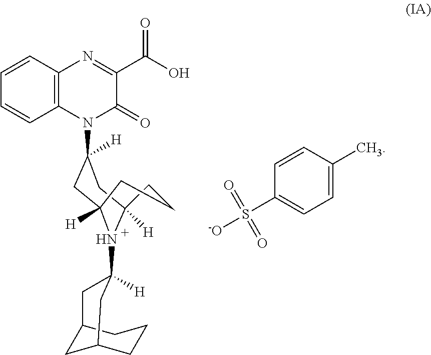

In some embodiments, the compound of formula (IA) can be administered orally, parenterally, intravenously, intramuscularly, buccally, or transdermally. In some embodiments, the compound of formula (IA) can be administered orally. In some embodiments, the effective amount of the compound of formula (IA) can be about 0.10 mg to about 10 mg. In some embodiments, the compound of formula (IA) can be administered once daily. In some embodiments, the compound of formula (IA) can be administered at nighttime. In some embodiments, the compound of formula (IA) can be administered prior to bedtime. In some embodiments, the compound of formula (IA) can be administered twice daily. In some embodiments, the compound of formula (IA) can be administered approximately every 12 hours.

In some embodiments, the method can comprise administering a first effective amount of the compound of formula (I) or pharmaceutically acceptable salt thereof during daytime, and administering a second effective amount of the compound of formula (I) or pharmaceutically acceptable salt thereof at nighttime prior to bedtime of the human subject. In some embodiments, the first effective amount can be a therapeutically effective amount and is the same as the second effective amount.

In some embodiments, the first effective amount and the second effective amount can be different. In some embodiments, the second effective amount can be about 2-fold greater than the first effective amount. In some embodiments, the second effective amount can be about 10-fold greater than the first effective amount.

In some embodiments, the administration of the compound of formula (I) or a pharmaceutically acceptable salt thereof can increase micturition pressure threshold in the human subject by about 30% to 80%. In some embodiments, the method can further comprises administering an effective amount of an antimuscarinic agent to the human subject. In some embodiments, the antimuscarinic agent can be oxybutynin, tolterodine, trospium, solifenacin, darifenacin, or a pharmaceutically acceptable salt of any of the foregoing.

The present disclosure provides the additional embodiments.
A) A method of treating or preventing overactive bladder syndrome in a human subject in need of such treatment, comprising administering to the human subject a therapeutically effective amount of a compound of formula (I):

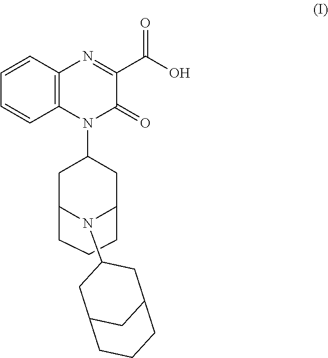 [SMILES: O=C(O)c1nc2ccccc2n(C2CC3CCCC(C2)N3C2CC3CCCC(C3)C2)c1=O]

or a pharmaceutically acceptable salt thereof.
B) The method of Embodiment A, wherein the compound is a compound of formula (I′):

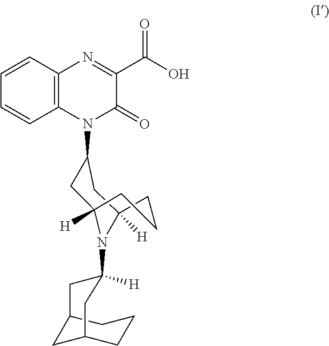

or a pharmaceutically acceptable salt thereof.
C) The method of Embodiment A or Embodiment B, wherein the method comprises administering a pharmaceutically acceptable salt of the compound, wherein the salt is selected from the group consisting of sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, and p-toluenesulfonate salts.
D) The method of any one of Embodiments A-C, wherein the method comprises administering a p-toluenesulfonic acid salt, a sulfate salt, a phosphoric acid salt, or a hydrochloride salt of the compound.
E) The method of any one of Embodiments A-D, wherein a p-toluenesulfonic acid salt of the compound is administered.
F) The method of any one of Embodiments A-E, wherein the method comprises administering a compound of formula (IA):

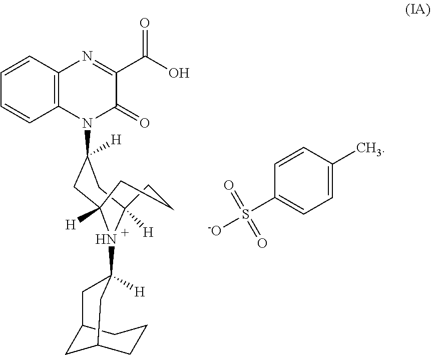

G) The method of any one of Embodiments A-F, wherein urinary frequency in the human subject is reduced.
H) The method of any one of Embodiments A-F, wherein episodes of nocturia are reduced in the human subject.
I) A method of reducing nocturia in a human subject in need of such treatment, comprising administering to the human subject a therapeutically effective amount of a compound of formula (I):

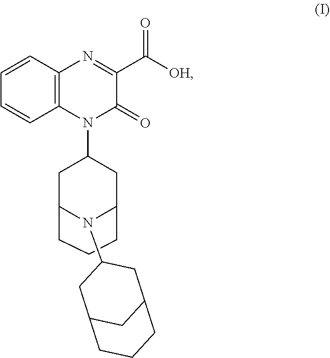 [SMILES: O=C(O)c1nc2ccccc2n(C2CC3CCCC(C2)N3C2CC3CCCC(C3)C2)c1=O]

or a pharmaceutically acceptable salt thereof.

J) The method of Embodiment I, wherein the compound is a compound of formula (I′):

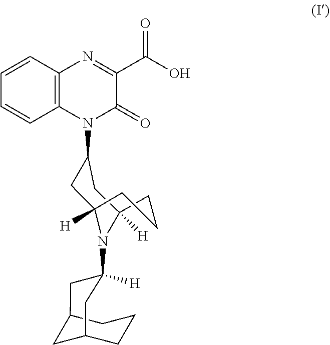

or a pharmaceutically acceptable salt thereof.
K) The method of Embodiment I or Embodiment J, wherein the method comprises administering a pharmaceutically acceptable salt of the compound, wherein the salt is selected from the group consisting of sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, and p-toluenesulfonate salts.
L) The method of any one of Embodiments I-K, wherein the method comprises administering a p-toluenesulfonic acid salt, a sulfate salt, a phosphoric acid salt, or a hydrochloride salt of the compound.
M) The method of any one of Embodiments I-L, wherein a p-toluenesulfonic acid salt of the compound is administered.
N) The method of Embodiment M, wherein the method comprises administering a compound of formula (IA):

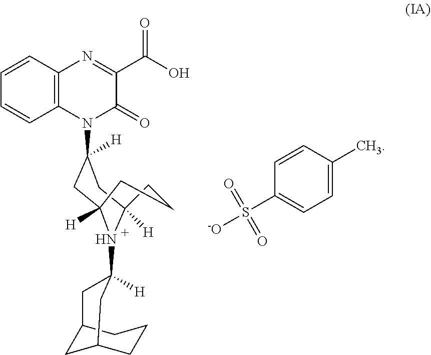

O) The method of any one of Embodiments A-N, wherein the compound or a pharmaceutically acceptable salt thereof is administered orally, parenterally, intravenously, intramuscularly, buccally, or transdermally.
P) The method of any one of Embodiments A-O, wherein the compound or a pharmaceutically acceptable salt thereof is administered orally.
Q) The method of any one of Embodiments A-P, wherein the therapeutically effective amount of the compound or a pharmaceutically acceptable salt thereof is from about 0.001 mg to about 300 mg.
R) The method of any one of Embodiments A-Q, wherein the therapeutically effective amount of the compound or a pharmaceutically acceptable salt thereof is from about 0.10 mg to about 10 mg.
S) The method of Embodiment R, wherein the compound or a pharmaceutically acceptable salt thereof is a compound of formula (IA):

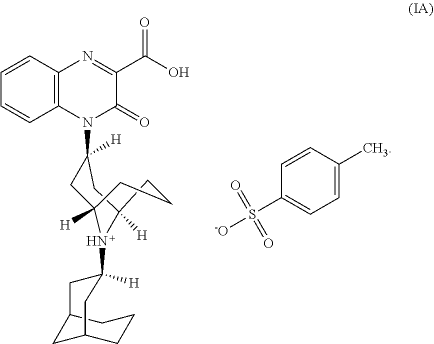

T) The method of any one of Embodiments A-S, wherein the compound or a pharmaceutically acceptable salt thereof is administered once daily.
U) The method of any one of Embodiments A-T, wherein the compound or a pharmaceutically acceptable salt thereof is administered at night.
V) The method of Embodiment U, wherein the compound or a pharmaceutically acceptable salt thereof is administered prior to bedtime.
W) The method of any one of Embodiment P-S, wherein the compound or a pharmaceutically acceptable salt thereof is administered twice daily.
X) The method of Embodiment W, wherein the compound or a pharmaceutically acceptable salt thereof is administered approximately every 12 hours.
Y) The method of Embodiment W or Embodiment X, wherein the method comprises administering a first therapeutically effective amount of the compound or a pharmaceutically acceptable salt thereof during daytime, and administering a second therapeutically effective amount at nighttime prior to bedtime of the human subject.
Z) The method of Embodiment Y, wherein the first therapeutically effective amount and the second therapeutically effective amount are the same.
AA) The method of Embodiment Y, wherein the first therapeutically effective amount and the second therapeutically effective amount are different.
BB) The method of Embodiment AA, wherein the second therapeutically effective amount is about 2-fold greater than the first therapeutically effective amount.
CC) The method of Embodiment AA, wherein the second therapeutically effective amount is about 10-fold greater than the first therapeutically effective amount.
DD) The method of any one of Embodiments A-CC, wherein the administration of the compound or a pharmaceutically acceptable salt thereof increases a micturition pressure threshold in the human subject by about 30% to 80%.
EE) The method of any one of Embodiments A-DD, further comprising administering an effective amount of an antimuscarinic agent to the human subject.
FF) The method of Embodiment EE, wherein the antimuscarinic agent is oxybutynin, tolterodine, trospium, solifenacin and darifenacin, or a pharmaceutically acceptable salt of any of the foregoing.
GG) A method of treating or preventing overactive bladder syndrome in a human subject in need of such treatment, comprising administering to the human subject a therapeutically effective amount of a compound of formula (I):

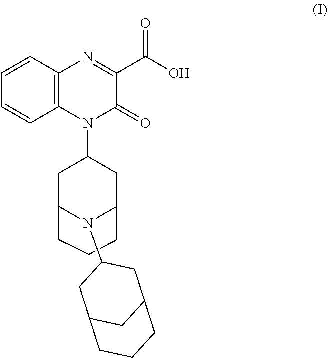 [SMILES: O=C(O)c1nc2ccccc2n(C2CC3CCCC(C2)N3C2CC3CCCC(C3)C2)c1=O]

or a pharmaceutically acceptable salt thereof, wherein the patient additionally suffers from a sleep disorder.
HH) The method of Embodiment GG, wherein the compound is a compound of formula (I′):

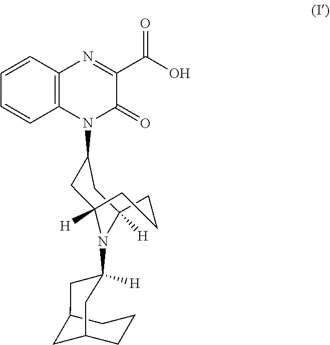

or a pharmaceutically acceptable salt thereof.
II) The method of Embodiment GG or Embodiment HH, wherein the method comprises administering a pharmaceutically acceptable salt of the compound, wherein the salt is selected from the group consisting of sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, and p-toluenesulfonate salts.
JJ) The method of any one of Embodiments GG-II, wherein the pharmaceutically acceptable salt is a p-toluenesulfonic acid salt, a sulfate salt, a phosphoric acid salt, or a hydrochloride salt.
KK) The method of any one of Embodiments GG-JJ, wherein the pharmaceutically acceptable salt a p-toluenesulfonic acid salt.
LL) The method of any one of Embodiments GG-KK, wherein method comprises administering the compound of formula (IA):

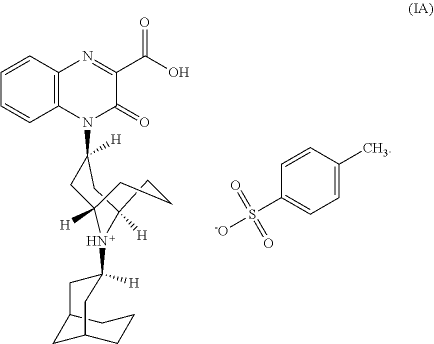

MM) The method of any one of Embodiments GG-LL, wherein the human subject is a female of 50 years of age or older.
NN) A method of treating a sleep disorder in a patient, comprising administering to the patient a therapeutically effective amount of a compound of formula (I):

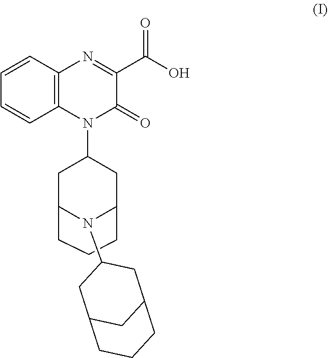 [SMILES: O=C(O)c1nc2ccccc2n(C2CC3CCCC(C2)N3C2CC3CCCC(C3)C2)c1=O]

or a pharmaceutically acceptable salt thereof, wherein said patient also suffers urinary incontinence.
OO) The method of Embodiment NN, wherein the compound is a compound of the formula (I′):

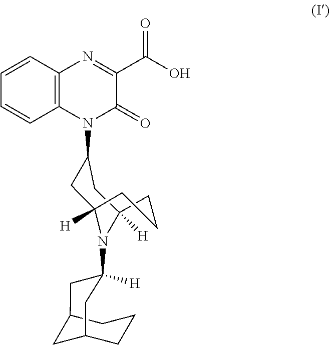

or a pharmaceutically acceptable salt thereof.
PP) The method of Embodiment NN or Embodiment OO, wherein the method comprises administering a compound of formula (IA):

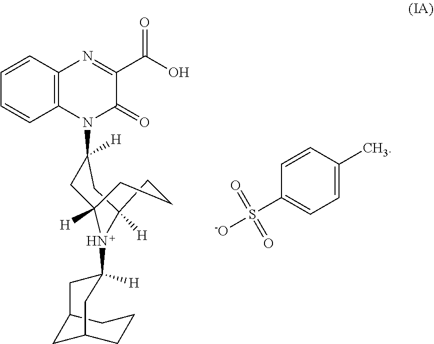

QQ) The method of any one of Embodiments NN-PP, wherein said sleep disorder is an insomnia condition, a hypersomnia condition, a circadian rhythm sleep-wake disorder, an alcohol-induced sleep disorder, insomnia associated with alcohol cessation, or any combination thereof.
RR) A method of treating overactive bladder syndrome in a human subject in need thereof, comprising administering to the human subject an effective amount of a compound of formula (I):

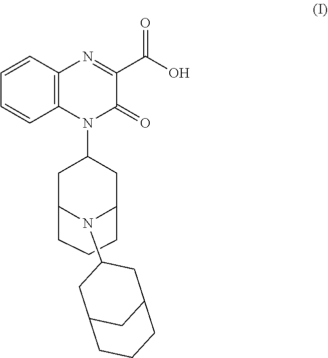 [SMILES: O=C(O)c1nc2ccccc2n(C2CC3CCCC(C2)N3C2CC3CCCC(C3)C2)c1=O]

or a pharmaceutically acceptable salt thereof,
   wherein the human subject has a mild or mild to moderate renal impairment.
SS) The method of Embodiment RR, wherein the human subject with mild renal impairment has an estimated glomerular filtration rate (eGFR) of about 60 mL/min to about 89 mL/min.
TT) The method of Embodiment RR, wherein the human subject with mild to moderate renal impairment has an eGFR of about 45 mL/min to about 59 mL/min.
UU) The method of any one of claims Embodiments RR-TT, wherein administering the compound of formula (I) to the subject results in at least a mean AUC, Cₘₐₓ, Tₘₐₓ, T_{1/2}, or CL/F that is not statistically different from a corresponding mean AUC, Cₘₐₓ, Tₘₐₓ, T_{1/2}, or CL/F in a similarly situated human subject with no renal insufficiency.
VV) The method of any one of Embodiments RR-UU, wherein administering the compound of formula (I) to the human subject results in at least a mean Ae, Fe, or CL_{R} that is not statistically different from a corresponding mean Ae, Fe, or CL_{R} in a similarly situated human subject with no renal insufficiency.
WW) The method of any one of Embodiments RR-VV, wherein the compound is a compound of formula (I′):

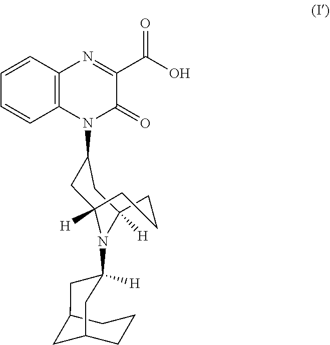

or a pharmaceutically acceptable salt thereof.
XX) The method of any one of Embodiments RR-WW, wherein the method comprises administering a pharmaceutically acceptable salt of the compound of formula (I), wherein the salt is selected from the group consisting of sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, and p-toluenesulfonate salts.
YY) The method of any one of Embodiments RR-XX, wherein the method comprises administering a p-toluenesulfonic acid salt, a sulfate salt, a phosphoric acid salt, or a hydrochloride salt of the compound of formula (I).
ZZ) The method of any one of Embodiments RR-YY, wherein a p-toluenesulfonic acid salt of the compound of formula (I) is administered.
AAA) The method of any one of Embodiments RR-ZZ, wherein the compound of formula (I) or the pharmaceutically acceptable salt thereof is a compound of formula (IA):

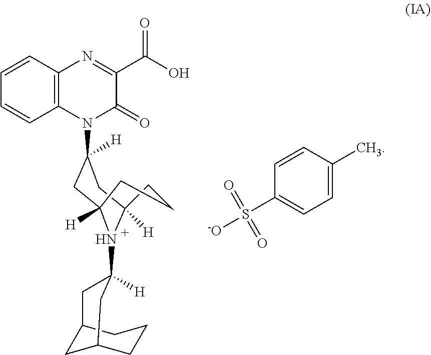

BBB) The method of Embodiment AAA, wherein the compound of formula (IA) is administered orally, parenterally, intravenously, intramuscularly, buccally, or transdermally.
CCC) The method of Embodiment AAA or Embodiment BBB, wherein the compound of formula (IA) is administered orally.
DDD) The method of Embodiment CCC, wherein the effective amount of the compound of formula (IA) is about 0.10 mg to about 10 mg.
EEE) The method of any one of Embodiments AAA-DDD, wherein the compound of formula (IA) is administered once daily.
FFF) The method of any one of Embodiments AAA-EEE, wherein the compound of formula (IA) is administered at nighttime.
GGG) The method of any one of Embodiments AAA-FFF, wherein the compound of formula (IA) is administered prior to bedtime.
HHH) The method of any one of Embodiments AAA-DDD, wherein the compound of formula (IA) is administered twice daily.
III) The method of Embodiment HHH, wherein the compound of formula (IA) is administered approximately every 12 hours.
JJJ) The method of Embodiment HHH or Embodiment III, wherein the method comprises administering a first effective amount of the compound of formula (I) or pharmaceutically acceptable salt thereof during daytime, and administering a second effective amount of the compound of formula (I) or pharmaceutically acceptable salt thereof at nighttime prior to bedtime of the human subject.
KKK) The method of Embodiment JJJ, wherein the first effective amount is a therapeutically effective amount and is the same as the second effective amount.
LLL) The method of Embodiment JJJ, wherein the first effective amount and the second effective amount are different.
MMM) The method of Embodiment LLL, wherein the second effective amount is about 2-fold greater than the first effective amount.
NNN) The method of Embodiment LLL, wherein the second effective amount is about 10-fold greater than the first effective amount.
OOO) The method of any one of Embodiments AAA-NNN, wherein the administration of the compound of formula (I) or a pharmaceutically acceptable salt thereof increases micturition pressure threshold in the human subject by about 30% to 80%.
PPP) The method of any one of Embodiments AAA-OOO, further comprising administering an effective amount of an antimuscarinic agent to the human subject.
QQQ) The method of Embodiment PPP, wherein the antimuscarinic agent is oxybutynin, tolterodine, trospium, solifenacin, darifenacin, or a pharmaceutically acceptable salt of any of the foregoing.

### EXAMPLES

### Example 1: Isovolumetric Model to Measure the Effect of the Compound of Formula (IA) on OBS

In rats, under isovolumetric condition, bladder distension induced rhythmic bladder contractions (RBC) (Aizawa et al, (2015) Effects of L-arginine, mirabegron, and oxybutynin on the primary bladder afferent nerve activities synchronized with reflexic, rhythmic bladder contractions in the rat. Neurourology & Urodynamics 34:368-374). This isovolumetric model allows the evaluation of compound on RBC. It is a closed system where the bladder is slowly infused through a urethral catheter with saline until the occurrence of spontaneous rhythmic bladder contractions.

The aim of this study was to evaluate the effects of intravenous administration of the compound of formula (IA) on cystometric parameters in the isovolumetric model in anesthetized female rats. Test substance effects were compared to that of the reference substance, tolterodine, an antimuscarinic receptor, marketed for overactive bladder in humans.

### Study Design

Protocol:
Animals were anesthetized with urethane (i.p.).
Ureters were ligated and sectioned close to the kidneys.
Intravesical catheter was inserted into the bladder though the urinary meatus.
Urinary meatus was closed.
A catheter was inserted into the jugular vein for compound administration.
Bladder was filled with saline until the occurrence of RBC.
After 30 min basal period, compounds were administered as described below.
The effect of the test substances was followed by 1-hour post administration.

### Experimental Groups:

Three (3) experimental groups were included as described in Table 1 below:

TABLE 1

| Group | Treatment (i.v.) | n |
|---|---|---|
| 1 | Vehicle (20% 2-hydroxypropyl-beta-cyclodextrin) | 10 |
| 2 | Compound of formula (IA) (3 mg/kg) | 10 |
| 3 | Tolterodine (1 mg/kg) | 10 |

Before the experiment starts, animals were randomly assigned to treatment groups. Randomization was designed to have at least one animal of each group on each experimental day. At the end of the experiments the excluded animals were replaced.

### Animals:

All experiments were conducted in accordance with the European Community Council Directive 2010/63/UE and the French Ministry for Agriculture, Agrifood and Forestry Decree 2013-118.

Female Sprague-Dawley rats were acclimated to the laboratory conditions for at least 3 days before the start of experiments. Animals were housed in groups of 3 in polysulfone type Sealsafe plus 1291H cages (Tecniplast, Lyon, France) on a bed of wood chips (Souralit, Girona, Spain) with free access to food (Rodent Maintenance Diet A04/10 from Safe) and water (0.2 μm filtered water). Species appropriate environmental enrichment (Aspen brick, Plexx, Uden, Netherlands) was added in the cages. The animal house was maintained under artificial lighting (12 h) between 7:00 am to 7:00 pm in a controlled ambient temperature of 22±2° C., and relative humidity maintained at 55±10%.

### Test Substances

The appropriate amount of the compound of formula (I) was weighed and dissolved in vehicle to obtain the dosing solution with the target concentration at 3 mg/mL (as free base). Dosing formulation (3 mL) was stored in the fridge for a maximum of 3 days. The mother solution was used as is for the 3 mg/kg dose.

The 20% (w/v) 2-hydroxypropyl-beta-cyclodextrin (HP-β-CD) aqueous solution was prepared by dissolving 10 g of HP-β-CD (Sigma-Aldrich, Saint-Quentin Fallavier, France, batch n° BCBV0722) in 50 mL of water for injection (WFI). Vehicle was prepared for one week.

Tolterodine was prepared fresh on the day of administration at a final concentration of 1 mg/mL (free base form). Appropriate mass of tolterodine was weighed and dissolved in vehicle (20% HP-β-CD) at room temperature.

Urethane was purchased from Sigma-Aldrich. Dolethal® was purchased from Vetoquinol via Centravet (Lapalisse, France). Saline was purchased from B-Braun via Centravet. WFI was supplied by Cooper (Melun, France).

### Study Protocols

Rats were anesthetized with an intraperitoneal administration of urethane (1 g/kg). The ureters were ligated and sectioned close to the kidneys. A catheter (0.30 and 0.70 mm of inner and outer diameters, respectively) was inserted through the urinary meatus into the bladder before urethral ligature. Another catheter (0.58 and 0.96 mm of inner and outer diameters, respectively) was inserted into the jugular vein for intravenous (i.v.) administration.

For all group, animals were treated by i.v. route (1 mL/kg) (slow bolus) after the basal period.

Cystometry assessment:

The vesical catheter was connected via a T-tube to a strain gauge (to measure intra-vesical pressure) and to a syringe. Isovolumetric bladder contractions were induced by stepwise injections of physiological saline at room temperature (100 μL every 5 min) until stable RBC occurred. Vesical pressure was continuously recorded. After a 30 min control period (basal values), substances or vehicle were i.v. administered. The effects of substances were followed during 60 min after administration. At the end of the experiments, animals were sacrificed by cervical dislocation.

### Laboratory Equipment:

Animals were weighed using a LS620C balance (Precisa, Dietikon, Switzerland).

Surgery was performed using SX45 binocular microscope (Fisher Scientific, Illkirch, France). Thermo-regulated system (TCAT-2LV Controller, Physitemp Instruments, Clifton, NJ, USA) set up at 37° C. was used during surgery.

For cystometry, intravesical pressure was measured with a strain gauge MX960P1 (Smiths Medical, Rungis, France) and recorded continuously using a PowerLab/8-30 or 8-35 data acquisition system (ADInstruments Pty Ltd) and LabChart® software version 7.3.7.

### Expression and Analysis of Results

The following parameters were analyzed:
Amplitude of RBC (mmHg)
Frequency of RBC (number/30 min)
Time of inhibition (sec)

As demonstrated in FIG. 8, cystometric parameters were analyzed for each RBC during the 30 minute period before administration (basal values) and the whole 60 min period following administration. The % variation from the basal values were calculated for RBC frequency and amplitude.

Statistical analysis:

Statistical analysis and graphs were performed using GraphPad Prism® (GraphPad Software Inc., La Jolla, CA, USA). A p value <0.05 was accepted for statistical significance. Basal values of RBC frequency and amplitude of all groups were compared using one way ANOVA followed by Turkey's test Basal period and treated period of RBC frequency and amplitude, for each group, were compared using paired Student t-test or Wilcoxon test The % variation from basal values of RBC frequency, time of inhibition or amplitude of inhibition of tolterodine and the compound of formula (IA) were compared to vehicle group using Kruskal Wallis or one-way ANOVA followed by Dunn's or Dunnett's tests.

### Results

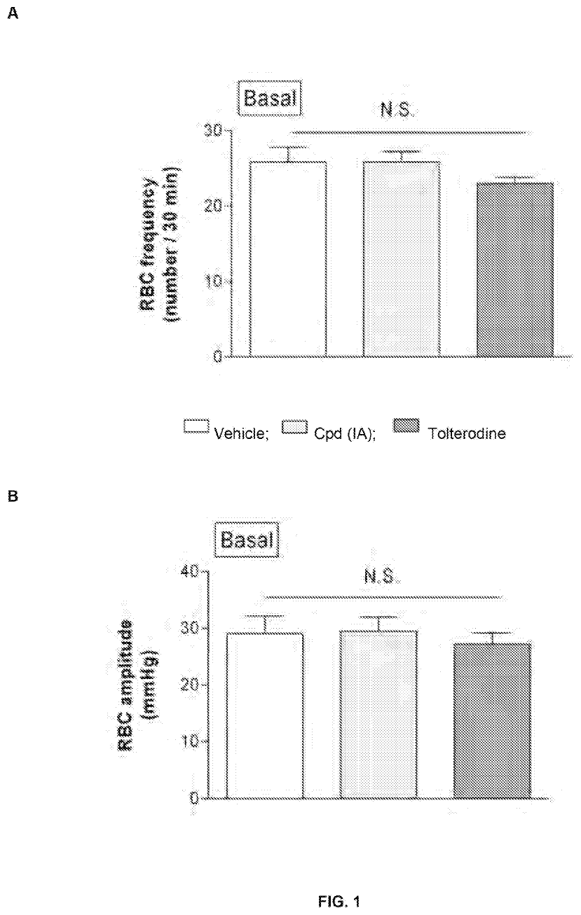

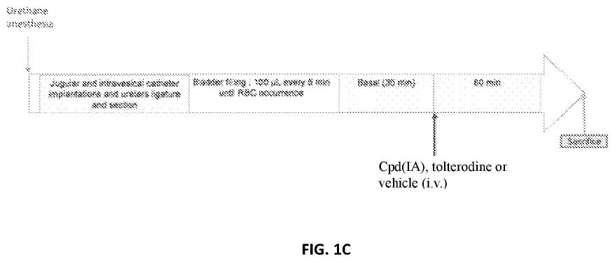

The basal values of RBC frequency in all experimental groups were not significantly different (p>0.05, FIG. 1A). The basal values of RBC frequency were 25.90±1.91, 25.90±1.36 and 23.00±0.87 within 30 min for the vehicle, the compound of formula (IA), and tolterodine groups, respectively (Tables 2 to 4).

The basal values of RBC amplitude in all experimental groups were not significantly different (p>0.05, FIG. 1B). The basal values of RBC amplitude were 29.13±3.02, 29.49±2.46 and 27.22±2.03 mmHg for the vehicle, the compound of formula (IA), and tolterodine groups, respectively (Tables 2 to 4).

TABLE 2
Effects of vehicle (1 mL/kg, i.v., n = 10) on RBC frequency,
RBC amplitude and time of inhibition in anesthetized female rats
Group 1: Vehicle

| | | Basal | Treated period | % variation |
|---|---|---|---|---|
| Parameters | | −30/0 min | 0/60 min | 0/60 min |
| Frequency | Mean | 25.90 | 18.50 | −28.18 |
| (nb of RBC/30 min) | SEM | 1.91 | 2.42 | 7.30 |
| RBC amplitude | Mean | 29.13 | 27.23 | −5.18 |
| (mmHg) | SEM | 3.02 | 2.54 | 2.12 |
| Inhibition | Mean | 422.89 | | |
| time (sec) | SEM | 113.60 | | |

TABLE 3
Effects of Cpd (IA) (3 mg/kg, i.v., n = 8-10) on RBC frequency,
RBC amplitude and time of inhibition in anesthetized female rats
Group 2: Cpd (IA) 3 mg/kg

| | | Basal | Treated period | % variation |
|---|---|---|---|---|
| Parameters | | ~30/0 min | 0/60 min | 0/60 min |
| Frequency | Mean | 25.90 | 3.05 | ~88.57 |
| (nb of RBC/30 min) | SEM | 1.36 | 1.02 | 3.68 |
| RBC amplitude | Mean | 29.49 | 27.57 | 1.25 |
| (mmHg) | SEM | 2.46 | 2.31 | 4.53 |
| Inhibition | Mean | 2860.51 | | |
| time (sec) | SEM | 272.38 | | |

Note:
for Cpd (IA) group, only 8 rats are averaged for RBC amplitude due to the absence of RBC after administration.

TABLE 4
Effects of Tolterodine (1 mg/kg, i.v., n = 10) on RBC frequency,
RBC amplitude and time of inhibition in anesthetized female rats
Group 3: Tolterodine 1 mg/kg

| | | Basal | Treated period | % variation |
|---|---|---|---|---|
| Parameters | | −30/0 min | 0/60 min | 0/60 min |
| Frequency | Mean | 23.00 | 15.30 | −33.93 |
| (nb of RBC/30 min) | SEM | 0.87 | 2.72 | 10.93 |
| RBC amplitude | Mean | 27.22 | 18.56 | −30.83 |
| (mmHg) | SEM | 2.03 | 1.53 | 5.24 |
| Inhibition | Mean | 87.04 | | |
| time (sec) | SEM | 31.06 | | |

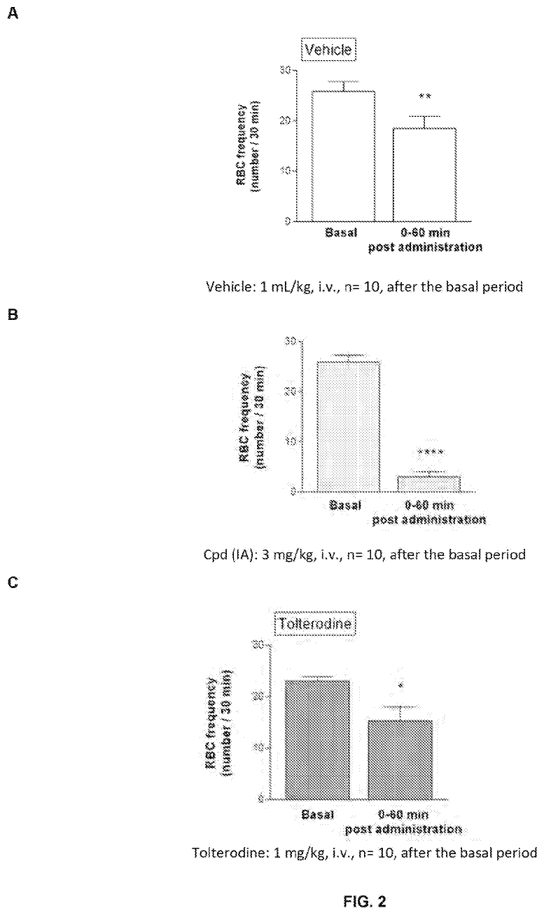

Vehicle (1 mL/kg, i.v.), the compound of formula (IA) (3 mg/kg, i.v.), and tolterodine (1 mg/kg, i.v.) significantly decreased RBC frequency (p<0.05 and p<0.0001, FIGS. 2A-B-C, Tables 2-4).

The values of RBC frequency during the 0/60 min post-treatment period were 18.50±2.42, 3.05±1.02 and 15.30±2.72 RBC/30 min after vehicle, the compound of formula (IA), and tolterodine treatment, respectively (FIGS. 2A-B-C, Tables 2-4).

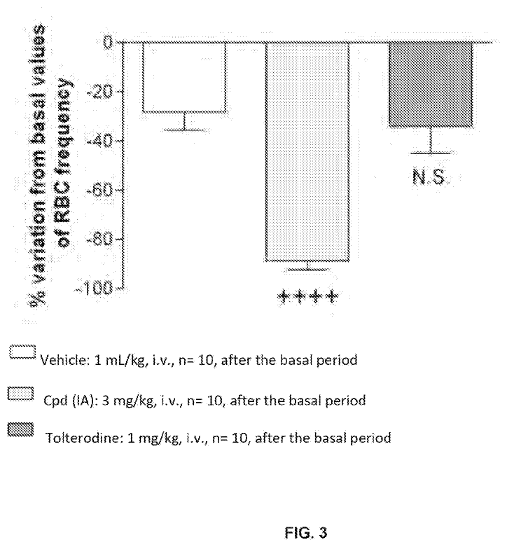

RBC frequency was significantly decreased after treatment of the compound of formula (IA) (3 mg/kg, i.v.) (p<0.0001, FIG. 3, Table 3), whereas tolterodine (1 mg/kg, i.v.) did not significantly modify RBC frequency (p>0.05, FIG. 3, table 4). The reduction of RBC frequency after treatment of the compound of formula (IA) reached-88.57±3.68% whereas the reduction after vehicle and tolterodine treatment were-28.18±7.30 and −33.93±10.93%, respectively (FIG. 3, Tables 2 to 4).

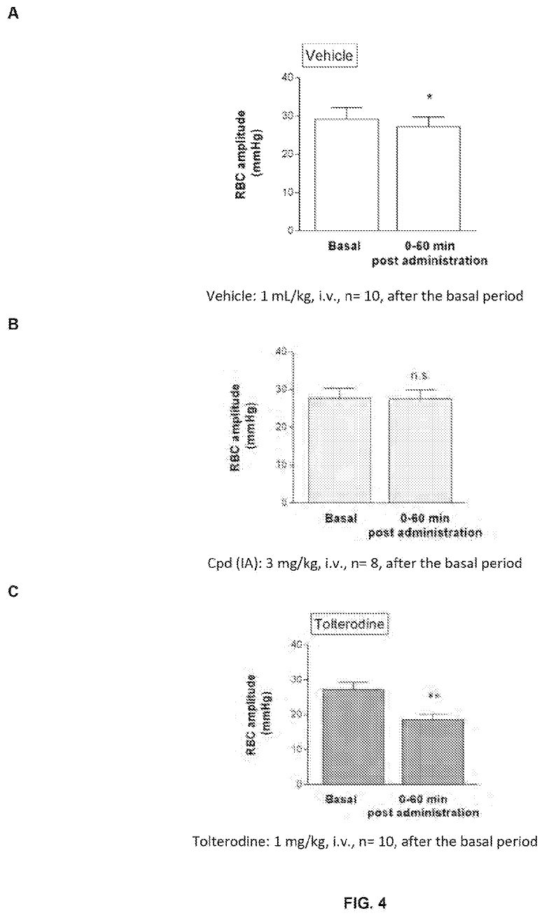

Compared to basal values, vehicle and tolterodine significantly reduced RBC amplitude (p<0.05 and p<0.01, FIGS. 4A-4C, Tables 2 to 4). No significant effect was observed on RBC amplitude after the compound of formula (IA) administration (p>0.05, FIG. 4B, Table 3).

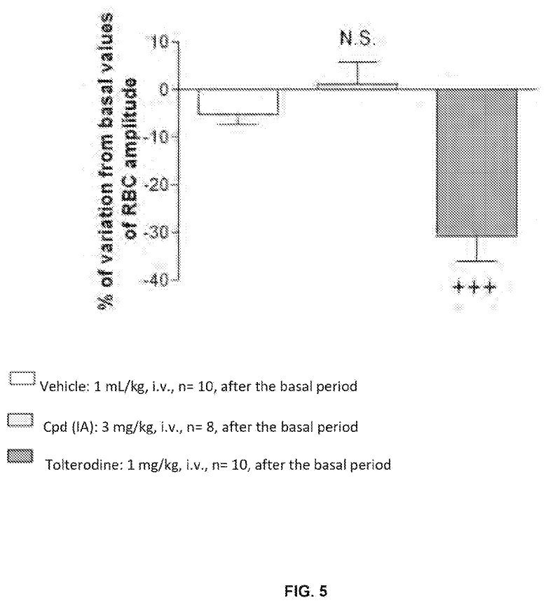

Compared to vehicle, tolterodine (1 mg/kg, i.v.) significantly reduced RBC amplitude (p<0.001, FIG. 5 & Tables 2 to 4) whereas the compound of formula (IA) (3 mg/kg) did not significantly modify RBC amplitude (p>0.05, FIG. 5 & Tables 2-3). The percentage of variation from basal values of RBC amplitude after tolterodine was −30.83±5.24 versus −5.18±2.12% for vehicle group.

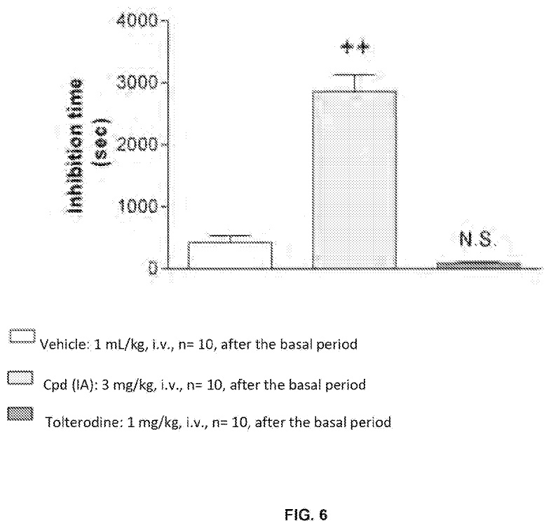

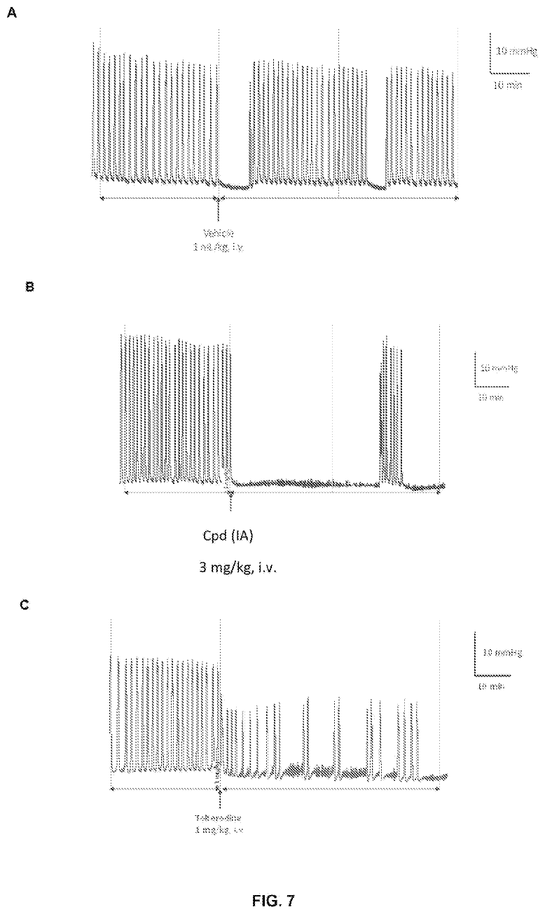

Compared to vehicle, the compound of formula (IA) (3 mg/kg, i.v.) significantly increased the time of inhibition (p<0.01, FIG. 6 & Table 3) whereas no significant difference on time of inhibition was observed after tolterodine treatment (p>0.05, FIG. 6 & Table 4). The times of inhibition were 422.89±113.60, 2860.51±272.38 and 87.04=31.06 sec for the vehicle, the compound of formula (IA) and tolterodine groups, respectively.

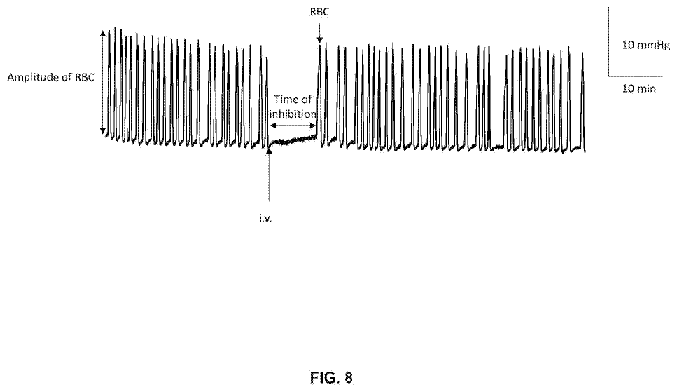

Typical recording of the effects of vehicle, the compound of formula (IA), and tolterodine are presented in FIGS. 7A, 7B and 7C, respectively.

The results in Example 1 show that the compound of Formula (IA) significantly decreased bladder afferent activity. On the other hand, tolterodine had no effect on afferent activity but effectively inhibited bladder efferent activity.

### Example 2: Pharmacokinetic Studies on the Compound of Formula (IA)

The urine concentrations were evaluated in humans (9 total) for up to 48 hours after administration of a single oral dose of the compound of formula (IA) (aka, Cpd (IA)) in the form of a methyl cellulose suspension. Urine samples (pooled serial samples of all urine voided) for determining concentrations of the compound of formula (IA) were collected for individual subjects at the following time intervals: 0 to 8 hours, 8 to 16 hours, 16 to 24 hours, 24 to 32 hours and 40 to 48 hours after administration. Individual subject and summaries of the urine concentration for the compound of formula (IA) by treatment and time interval are provided in Table 5, below.

TABLE 5
Individual subject and summaries of the urine concentration for Cpd (IA)

| | | Treatment/Concentration (nM) | | | | | | | |
|---|---|---|---|---|---|---|---|---|---|
| | | Cpd (IA) 0.2 mg | | Cpd (IA) 0.6 mg | | Cpd (IA) 2 mg | | Cpd (IA) 10 mg | |
| NOMTIME | Variable | Mean | SD | Mean | SD | Mean | SD | Mean | SD |
| 0 to 8 h | Urine Conc. | 2311 | 1736 | 1641 | 1196 | 10809 | 6322 | 30964 | 19832 |
| 8 to 16 h | Urine Conc. | 394 | 226 | 531 | 363 | 1440 | 768 | 14181 | 8646 |
| 16 to 24 h | Urine Conc. | BCL | BCL | BCL | BCL | 409 | 289 | 1004 | 805 |
| 24 to 32 h | Urine Conc. | BCL | BCL | BCL | BCL | BCL | BCL | 535 | 55 |
| 32 to 40 h | Urine Conc. | BCL | BCL | BCL | BCL | BCL | BCL | 240 | BCL |
| 40 to 48 h | Urine Conc. | BCL | BCL | BCL | BCL | BCL | BCL | BCL | BCL |

LLOQ = 100 ng/mL = 229 nM

In vitro activity against human nociception receptors; Ki=2.45 nM, EC₅₀=4.0 nM

Table 5 shows the mean urine concentration levels of the Compound of formula (IA) at particular time intervals and standard deviations after administration of particular doses (0.2 mg, 0.6 mg, 2 mg and 10 mg) of the Compound of formula (IA). Table 5 shows that even at the lowest dose of the compound of formula (IA) (0.2 mg), the urine concentration is orders of magnitude greater than the in vitro activity of the Compound of formula (IA) as measured by Ki and EC₅₀.

### Example 3: Effects of the Compound of Formula (Ia) on Cystometric Parameters in Female Rats (Sci Model)

Spinal cord injury (SCI) produces significant changes in urinary bladder function in multiple species including rat and human. In rats, SCI interrupts voluntary voiding and the reflex pathways that control bladder and sphincter function resulting in an areflexic bladder and the inability to void. Two weeks after spinal injury, micturition reflex reappears but there is a competition between bladder and urethral sphincter and a bladder distension inducing detrusor hyperreflexia. The development of a spinal reflex pathways that enable the rats to void demonstrating that neurotransmission reappears (Cheng et al., 1995, Yoshiyama et al., 1999).

The abnormal reflex pathways coupled with problems in the coordination between the bladder and sphincter lead to bladder hypertrophy, inefficient voiding and detrusor overactivity (de Groat & Yoshimura., 2010). SCI in rat, at the thoracic level, has been shown to increase non-voiding contractions (NVC) frequency and bladder capacity, and to decrease voiding efficiency (Yoshiyama et al., 1999, Kadekawa et al., 2017, Wada et al., 2017, Wada et al., 2018). SCI in rats is also characterized by bladder hypertrophy. Consequently, this model is widely used for the evaluation of substances dedicated to neurogenic bladder dysfunction. In addition, the β3-adrenoceptor agonist CL-316,243 was shown to significantly increase bladder capacity and to reduce the frequency of NVC (Beauval et al., 2015).

The aim of this study was to evaluate the effects of intragastric (i.g.) administration of the Compound of formula (IA) (“Cpd (IA)”) (30 mg/kg) on cystometric parameters in conscious SCI rats. Test substance effects were compared to those of the reference substance, the β3-adrenoceptor agonist mirabegron, marketed for overactive bladder.

### Study Design

### Protocol Design

At W-5, spinal cord injury was performed at T₈ level as described below.
Over W-5, rats were treated daily with gentamicin.
Over W-5 and W-4, bladder was emptied manually once a day.
From W-5 to W0, animals were weighed once per week.
At D-2 (W0), implantation of catheters was performed under isoflurane anaesthesia as described below.
At D0 (W0), cystometry was performed

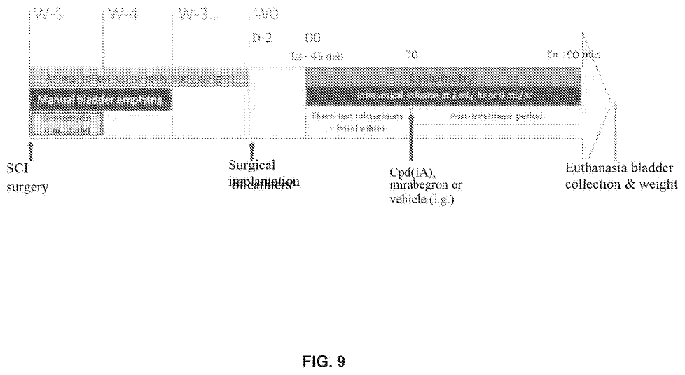

The scheme of the protocol design is provided in FIG. 9.

Experimental groups:

Four (4) experimental groups were included as described in the table below:

TABLE 6

| Group | Surgery | Treatment (i.g.) | n |
|---|---|---|---|
| 1 | Sham | Vehicle (5 mL/kg) | 12 |
| 2 | SCI | Vehicle (5 mL/kg) | 11 |
| 3 | SCI | Cpd (IA) (30 mg/kg) | 12 |
| 4 | SCI | Mirabegron (10 mg/kg) | 13 |

Before the experiment starts, animals were randomly assigned to treatment groups. Randomization was designed to have at least one animal of each group on each experimental day. At the end of the experiments, the follow up of animals was completed with missing animals.

Female Sprague-Dawley rats were acclimatized to the laboratory conditions for at least 3 days before the start of experiments. Animals were housed individually after SCI surgery for two weeks, then in group of 2 until catheter implantation and individually until cytometry in polysulfone type Sealsafe plus 1291H cages (Tecniplast, Lyon, France) on a bed of wood chips (Souralit, Girona, Spain) with free access to food (Rodent Maintenance Diet A04/10 from Safe) and water (0.2 μm filtered water). Species appropriate environmental enrichment (Aspen brick, Plexx, Uden, Netherlands) was added in the cages. The animal house was maintained under artificial lighting (12 h) between 7:00 am to 7:00 pm in a controlled ambient temperature of 22±2° C., and relative humidity maintained at 55±10%.

### Preparation of Test Materials:

A stock solution of the Compound of formula (IA) was prepared in vehicle at a final concentration of 6 mg/mL (free base form). Appropriate volume of vehicle was added to weighed the Compound of formula (IA) slowly in a porcelain mortar and powder was ground with a pestle until a suspension was obtained. Aliquots of suspension were made (1 aliquot/administration) and kept at +4° C. for a maximum of 3 days. On each experimental day suspension was allowed to equilibrate to room temperature for at least 30 min prior to administration.

Vehicle was 0.5% methyl-cellulose (MC). It was prepared in water for injection (WFI) and kept at 4° C. for one week. MC (batch n° SLBR8963V) was purchased from Sigma-Aldrich (Saint-Quentin Fallavier, France).

Mirabegron was prepared fresh on the day of administration at a final concentration of 2 mg/mL (free base form). Appropriate mass of mirabegron was weighed and dissolved in vehicle at room temperature.

### Study Protocol

SCI surgery:

Rats were anesthetized with isoflurane (3%). Body temperature was constantly maintained at 37° C. throughout the surgery by placing animals on a thermo-regulated hot plate. A laminectomy was performed and the spinal cord was sectioned with microscissors at T8 level. Care was taken to assure that the section was complete by verifying that a slight retraction of the two segments occurred. Then the muscle was sutured and the skin fixed with staples. The scar was cleaned by Vetedine. For the Sham group, the same surgery was performed without section of the spinal cord.

The urinary bladder was squeezed once a day for 14 days with a gentle massage of the abdomen until restoration of the micturition reflex. In addition, they were administered intramuscularly daily for one week with gentamicin (2 mg/mL, 0.2 ml/rat). Animals were euthanized if they reached humane endpoints: weight loss of greater than 20% of their body weight and/or abnormal behavioral changes indicating presence of pain and distress (prostration, self-mutilation, aggressiveness).

Surgery for cystomanometry:

Rats were anesthetized with isoflurane (3%). Body temperature was constantly maintained at 37° C. throughout the surgery by placing animals on a thermo-regulated hot plate. A polyethylene catheter (0.58 and 0.96 mm of inner and outer diameters, respectively) was implanted in the bladder through the dome for intravesical pressure recording. Another catheter (0.58 and 0.96 mm of inner and outer diameters, respectively) was implanted in the stomach for intra-gastric administration. Catheters were exteriorized at the scapular level. After surgery, each rat was housed individually up to the end of the protocol with free access to food and water.

Animals were held under partial restraint in a restraining device. Saline was infused into the bladder at a constant flow rate of 2 or 6 mL/h for sham or SCI rats, respectively. After at least 45 min (3 complete micturition cycles corresponding to basal values), test substances were administered by intra-gastric route. Then, intravesical pressure was recorded for 90 min.

At the end of the experiments, rats were sedated with Dolethal® (0.3-0.5 mL/rat of pentobarbital sodium at 182.2 mg/mL; i.p.) prior euthanasia by cervical dislocation. Then, bladder was collected and weighed.

### Results and Analysis

All raw data were entered into an Excel® spreadsheet. All data entered have been compared with raw data by two persons before data analysis. Results are expressed as mean values±standard error of the mean (s.e.m.).

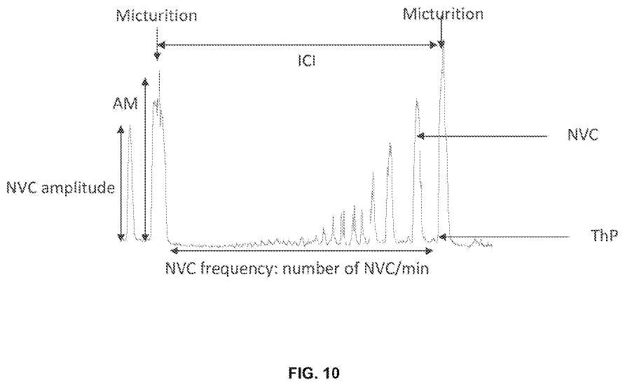

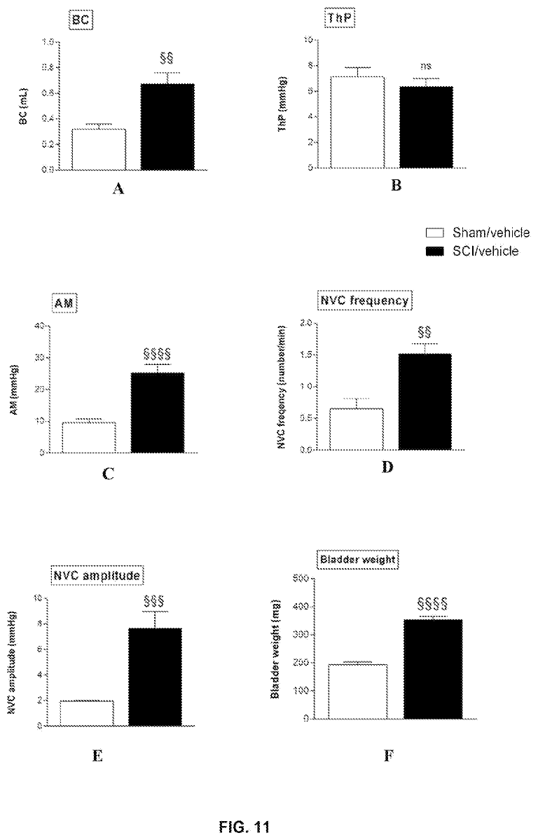

The following cystometric parameters were analyzed (see FIG. 10):
Amplitude of micturition (AM, mmHg).
Threshold pressure (ThP, mmHg).
Intercontraction interval (ICI, sec): time between basal pressure and ThP. ICI was not presented due to different infusion rate in sham and SCI rats. ICI was only analyzed to calculate the bladder capacity.
Bladder capacity (BC, mL) BC=ICI×infusion rate.
Amplitude of non voiding contractions (NVC, mmHg)
Frequency of NVC (number per min).

NVC were defined as an increase in intravesical pressure with an amplitude superior to 1.5 mmHg without urinary leakage.

Statistical analysis and graphs were performed using GraphPad Prism® (GraphPad Software Inc., La Jolla, CA, USA). Before carrying out any statistical test, the data were tested for normal distribution (Shapiro-Wilk normality test) and their variance evaluated (F test or Bartlett's test for two or more groups, respectively). The appropriate statistical test was consequently applied.

For each cystometric parameter and for each time period, the comparisons were performed as described below:
Basal values of each cystometric parameter of Sham/Vehicle and SCI/Vehicle groups were compared using unpaired student t test
Basal values of each cystometric parameter of all SCI groups were compared using one-way ANOVA or Kruskal-Wallis test
The 3 interval periods of 30 min post treatment of each cystometric parameter within the same group were compared to corresponding basal values using one-way ANOVA with repeated measures or Friedman test followed by Dunnett's or Dunn's post-test, respectively.
The 3 interval periods of 30 min post treatment of each cystometric parameter were compared between all SCI groups using one-way ANOVA or Kruskal Wallis test followed by Holm-Sidak's or Dunn's post-test versus Vehicle, respectively.

### Study Results:

The basal values of BC, AM, NVC frequency, NVC amplitude and bladder weight were significantly higher in SCI/Vehicle group compared to Sham/Vehicle group (p<0.01, FIG. 11). In contrast, basal values of ThP were not significantly different (p>0.05, FIG. 11).

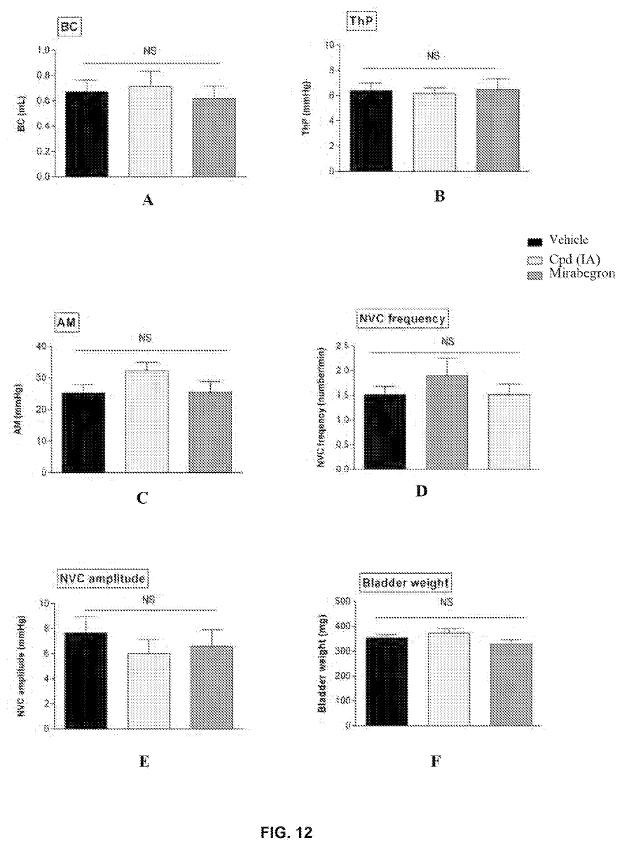

FIG. 12 shows that the basal values of BC, ThP, AM, NVC frequency and NVC amplitude were not significantly different in all experimental SCI groups (p>0.05).

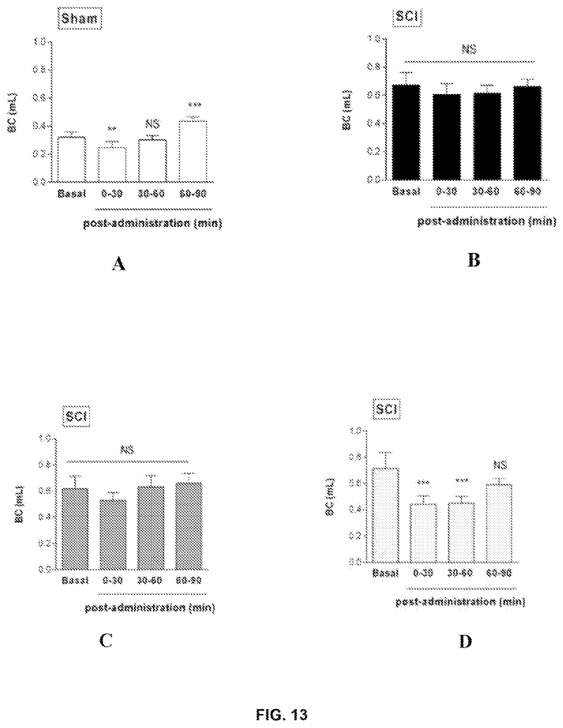

In sham rats, vehicle (5 mL/kg, i.g.) significantly decreased BC for the 0-30 min post-administration interval period and significantly increased BC for the last interval period (60-90 min) (p<0.01 and p<0.001, respectively, FIG. 13A).

In SCI rats, vehicle (5 ml/kg, i.g.) and mirabegron (10 mg/kg, i.g.) did not significantly modify BC (p>0.05, FIGS. 13B & C). In SCI rats, Cpd (IA) (30 mg/kg, i.g.) significantly decreased BC for the 0-30 and 30-60 min post-administration interval periods (p<0.001, FIG. 13D).

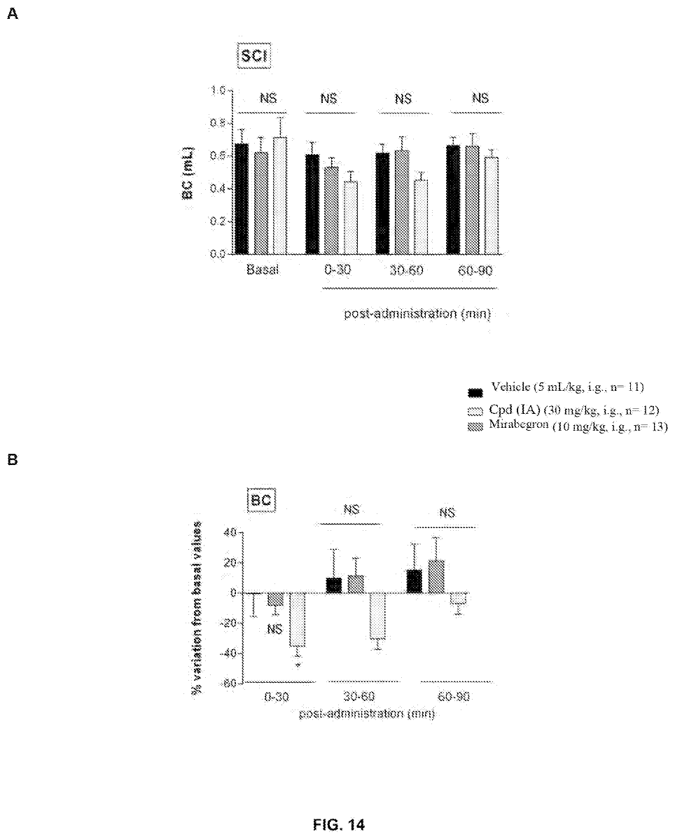

FIG. 14A shows that no significant difference in BC was observed in Mirabegron and Cpd (IA) groups (p>0.05, expressed in mL). FIG. 14B shows that Cpd (IA) (30 mg/kg, i.g.) significantly decreased BC for the 0-30 min post-administration interval period (p<0.05, expressed in % of variation from basal values). Indeed, the % of variation from basal values of BC was −35±7 and −0.4±15% in SCI/Cpd (IA) and SCI/Vehicle groups, respectively. After mirabegron treatment, BC was not significantly modified (p>0.05, FIG. 14B).

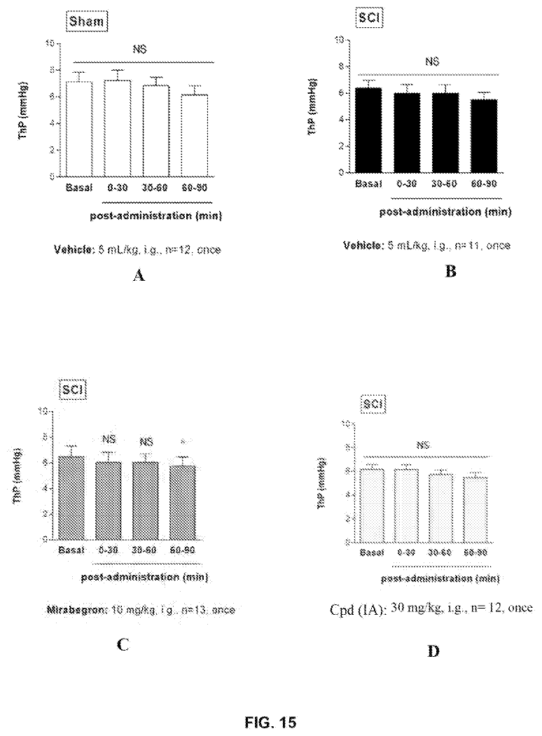

In sham and SCI rats, vehicle (5 mL/kg, i.g.) did not significantly modify ThP (p>0.05, FIGS. 15A & 15B). In SCI rats, mirabegron (10 mg/kg, i.g.) significantly decreased ThP for the 60-90 min post-administration interval period (p<0.05, FIG. 15C). In SCI rats, no significant effect on ThP was observed after Cpd (IA) treatment (p>0.05, FIG. 15D).

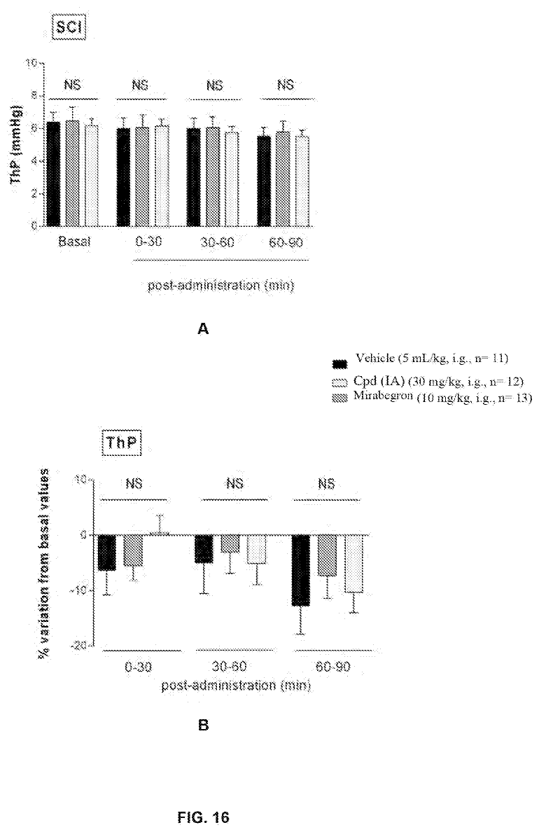

FIGS. 16A-B show that ThP was not significantly modified after mirabegron or Cpd (IA) treatment.

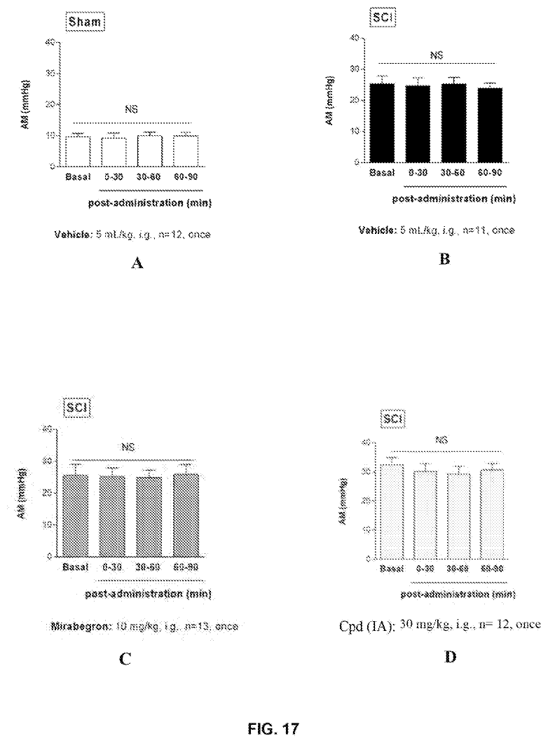

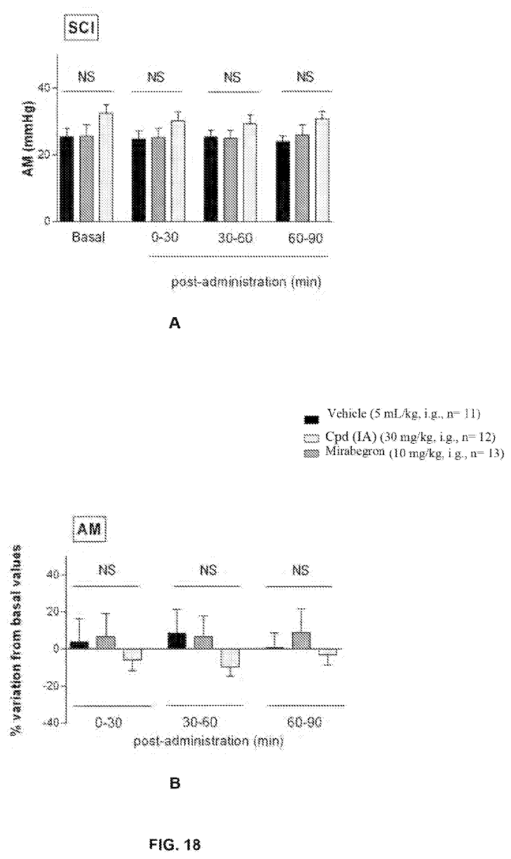

In sham and SCI rats, vehicle (5 mL/kg, i.g.) did not significantly affected AM (p>0.05, FIGS. 17A-B). In SCI rats, mirabegron (10 mg/kg, i.g.) and Cpd (IA) (30 mg/kg, i.g.) did not significantly modify AM (p>0.05, FIGS. 17C-D). AM was not significantly modified after mirabegron or Cpd (IA) treatment (p>0.05, FIGS. 18A-B).

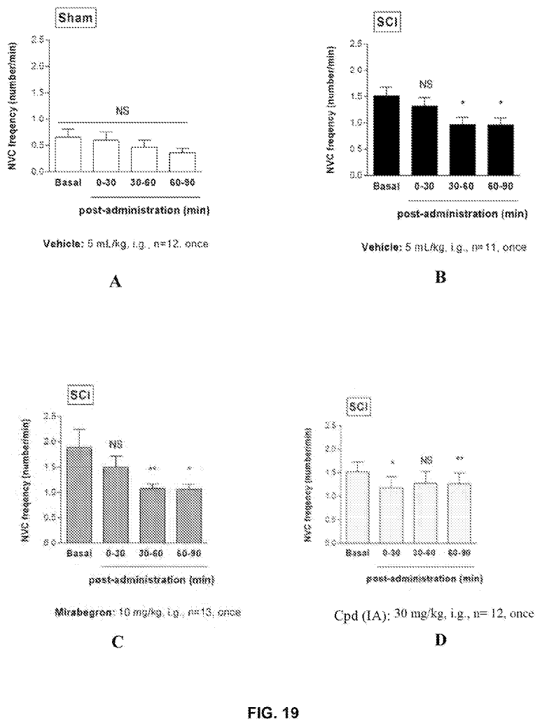

In sham rats, vehicle (5 mL/kg, i.g.) did not significantly modify NVC frequency (p>0.05, FIG. 19A). In SCI rats, vehicle (5 mL/kg, i.g.) significantly decreased NVC frequency for the 30-60 and 60-90 min interval periods (p<0.05, FIG. 19B). In SCI rats, mirabegron (10 mg/kg, i.g.) significantly decreased NVC frequency for the 30-60 and 60-90 min interval periods (p<0.01 and p<0.05, FIG. 19C). In SCI rats, Cpd (IA) (30 mg/kg, i.g.) significantly decreased NVC frequency for the 0-30 and 60-90 min interval periods (p<0.05 and p<0.01, FIG. 19D).

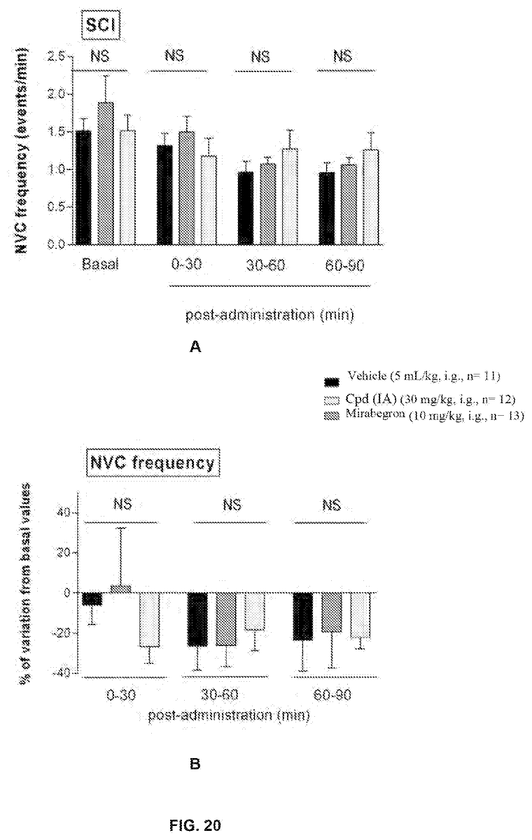

FIGS. 20A-B show that NVC frequency was not significantly modified after mirabegron or Cpd (IA) treatment.

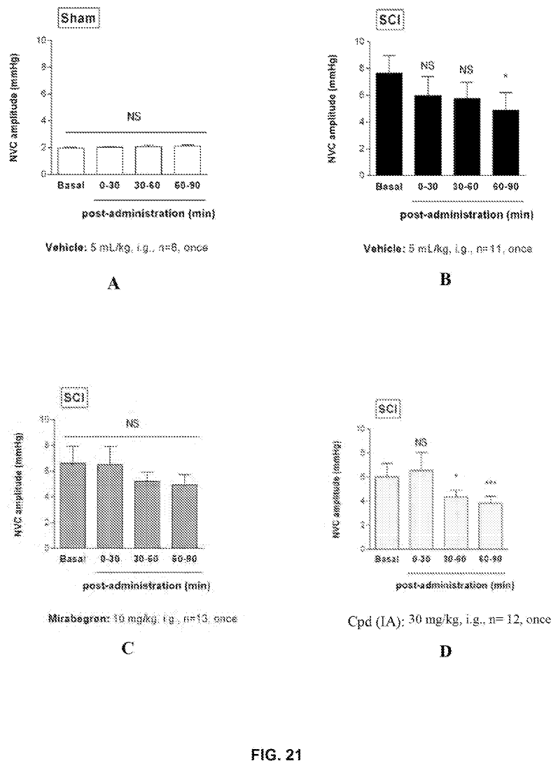

In sham rats, vehicle (5 mL/kg, i.g.) did not significantly modify NVC amplitude (p>0.05, FIG. 21A). In SCI rats, vehicle (5 mL/kg, i.g.) significantly decreased NVC amplitude for the 60-90 min post-administration interval period (p<0.05, FIG. 21B). In SCI rats, mirabegron (10 mg/kg, i.g.) did not significantly modified NVC amplitude (p>0.05, FIG. 21C). In SCI rats, Cpd (IA) (30 mg/kg, i.g.) significantly decreased NVC amplitude for the 30-60 and 60-90 min post-administration interval periods (p<0.05 and p<0.001, FIG. 21D).

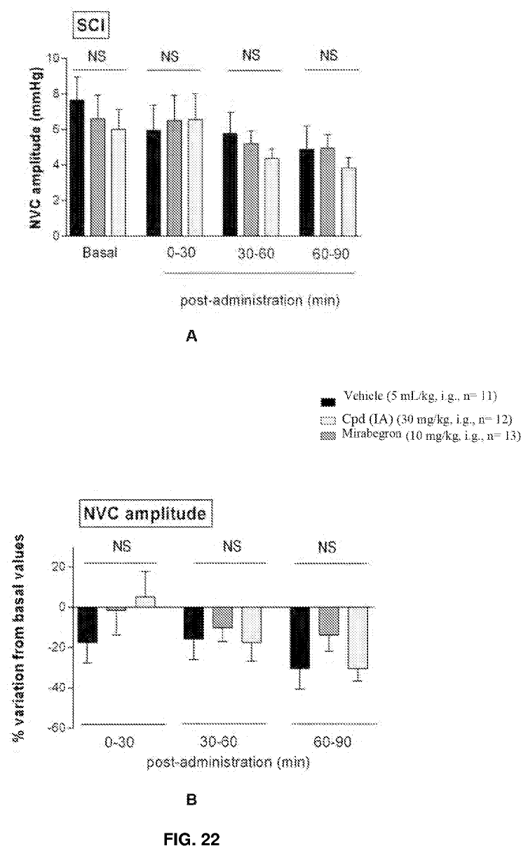

FIGS. 22A-B show that NVC amplitude was not significantly modified after mirabegron or Cpd (IA) treatment (p>0.05, Expressed in mmHg or in % variation from basal values).

In sham rats, compared to basal, vehicle (5 ml/kg, i.g.) decreased BC just after administration and increased BC for the 60-90 min interval period. The effects on BC after administration were probably due to an effect per se of the vehicle whereas the increase in BC for the 60-90 min post-administration interval period was due to a time effect. In SCI rats, vehicle (5 mL/kg, i.g.) had an effect on NVC frequency and NVC amplitude for the last post-administration interval periods confirming an effect per se of the vehicle or a time effect.

As expected, mirabegron (10 mg/kg, i.g.) slightly but significantly decreased NVC frequency compared to basal (Beauval et al., 2015).

The compound of formula (IA) (30 mg/kg, i.g.) significantly decreased NVC frequency compared to basal value for the 0-30 and 60-90 min post-administration interval periods. This is an expected effect for a substance dedicated to overactive bladder (OAB). Cpd (IA) also significantly reduced BC, compared to vehicle, for the 0-30 min post-administration interval period, which is not a desirable effect for the treatment of OAB.

### Example 4: Effects of the Compound of Formula (IA) on Cystometric Parameters in Female Rats (Boo Model)

Bladder outlet obstruction (BOO), in rats, is a well-known model of overactive bladder (OAB). In accordance with literature (Lluel et al., 1998), BOO induces bladder hypertrophy and bladder overactivity characterized by an increase in BC, AM and NVC frequency and amplitude. In this model, it was previously demonstrated that mirabegron, a β₃ receptor agonist, reduces the frequency of NVC (Gillespy et al., 2012).

The aim of this study was to evaluate the effects of intra-gastric (i.g.) administration of the Compound of Formula (IA) (“Cpd (IA)”) (30 mg/kg) on cystometric parameters in conscious BOO rats. Test substance effects were compared to those of the reference substance, mirabegron, marketed for overactive bladder.

### Study Protocol Design

At W-6, a partial ligature of the urethra was performed.
At W-6, during 3 consecutive days, rats were treated daily with gentamycin and Ketofen® The bladder was emptied manually once a day.
From W-6 to W-1 animals were monitored and weighed once per week.
At W0 (D-2), catheters were implanted under isoflurane anesthesia.
At D0, cystometry was performed.

### Animals and Test Substances:

Female Sprague-Dawley rats were acclimatized to the laboratory conditions for at least 3 days before the start of experiments. Animals were housed in groups of 2 or 3 and individually until cytometry in polysulfone type Sealsafe plus 1291H cages (Tecniplast, Lyon, France) on a bed of wood chips (Souralit, Girona, Spain) with free access to food (Rodent Maintenance Diet A04/10 from Safe) and water (0.2 μm filtered water). Species appropriate environmental enrichment (Aspen brick, Plexx, Uden, Netherlands) was added in the cages. The animal house was maintained under artificial lighting (12 h) between 7:00 am to 7:00 pm in a controlled ambient temperature of 22±2° C., and relative humidity maintained at 55±10%.

Appropriate amount of Cpd (IA) was weighed and dissolved in vehicle to obtain the dosing solution with the target concentration of 3 mg/mL (as free base). The mother solution was used as it for the 3 mg/kg dose.

Vehicle was 0.5% methyl-cellulose (MC). It was prepared in water for injection (WFI) and kept at 4° C. for one week. MC (batch n° SLBR8963V) was purchased from Sigma-Aldrich (Saint-Quentin Fallavier, France).

For intravenous administration, the 20% (w/v) 2-hydroxypropyl-beta-cyclodextrin (HP-β-CD) aqueous solution was prepared by dissolving 10 g of HP-β-CD (Sigma-Aldrich, batch n° BCBV0722) in 50 mL of WFI. Mirabegron (from Kemprotec) was prepared fresh on the day of administration at a final concentration of 2 mg/mL (free base form). Appropriate mass of mirabegron was weighed and dissolved in vehicle (0.5% MC) at room temperature.

Six (6) experimental groups were provided in the table below:

TABLE 7

| Group | Surgery | Treatment | n |
|---|---|---|---|
| 1 | Sham | Vehicle (5 mL/kg, i.g.) | 10 |
| 2 | BOO | Vehicle (5 mL/kg, i.g.) | 10 |
| 3 | BOO | Cpd (IA) (30 mg/kg, i.g.) | 10 |
| 4 | BOO | Mirabegron (10 mg/kg, i.g.) | 10 |
| 5 | BOO | Vehicle (1 mL/kg, i.v.) | 9 |
| 6 | BOO | Cpd (IA) (3 mg/kg, i.v.) | 9 |

### Results of Intra-Gastric (i.g.) Administration

The basal values of BC, AM, NVC frequency, NVC amplitude and bladder weight were significantly higher in BOO/Vehicle group compared to Sham/Vehicle group (p<0.05). In contrast, basal values of ThP were not significantly different (p>0.05)

The basal values of BC, ThP, AM, NVC frequency, NVC amplitude and bladder weight were not significantly different in all experimental BOO groups, including vehicle (5 mL/kg, i.g.), mirabegron (10 mg/kg, i.g.), and Cpd (IA) (30 mg/kg, i.g.).

For the 0-90 min entire period:

When compared to basal values, in sham and BOO rats, it was noted that vehicle had not significant effect on BC. In BOO rats, mirabegron did not significantly modify BC whereas Cpd (IA) significantly decreased BC. When compared to vehicle, expressed in mL, no significant difference in BC was observed in Mirabegron and Cpd (IA) groups. Expressed in % of variation from basal values, Cpd (IA) significantly decreased BC. The % of variation from basal values of BC was −13±6 and 11±6% in BOO/Cpd (IA) and BOO/Vehicle groups, respectively. After mirabegron treatment, BC was not significantly modified.

When compared to basal values, in sham and BOO rats, vehicle had no significant effect on ThP (p>0.05) whereas mirabegron and Cpd (IA) did not modify ThP (p>0.05). When compared to vehicle, expressed in mmHg, ThP was not significantly modified after mirabegron or Cpd (IA) treatment (p>0.05). Expressed in % variation from basal values, ThP was significantly decreased after Cpd (IA) treatment (p<0.05) whereas no significant effect was observed after mirabegron treatment (p>0.05).

When compared to basal values, in sham rats, vehicle significantly increased AM (p<0.01). In BOO rats, vehicle did not significantly affect AM (p>0.05) whereas mirabegron and Cpd (IA) did not significantly modify AM (p>0.05). When compared to vehicle, expressed in mmHg or in % variation from basal values, AM was not significantly modified after mirabegron or Cpd (IA) treatment (p>0.05).

When compared to basal values, in sham and BOO rats, vehicle did not significantly modify NVC frequency (p>0.05). In BOO rats, mirabegron significantly decreased NVC frequency (p<0.01). In BOO rats, Cpd (IA) did not affect NVC frequency (p>0.05). When compared to vehicle, expressed in number of NVC/min or in % variation from basal values, NVC frequency was not significantly modified after mirabegron or Cpd (IA) treatment (p>0.05).

When compared to basal values, in all experimental groups, vehicle, mirabegron and Cpd (IA) did not modify NVC amplitude (p>0.05). When compared to vehicle, expressed in number of mmHg, NVC amplitude was not significantly modified after mirabegron or Cpd (IA) treatment (p>0.05). Expressed in % variation from basal values, NVC amplitude was only significantly reduced after mirabegron treatment and remained unaffected by Cpd (IA) (p<0.05 and p>0.05).

For the 60-90 min interval period:

When compared to basal values, in sham rats, vehicle significantly increased BC (p<0.001). In BOO rats, vehicle did not significantly affect BC (p>0.05). In BOO rats, mirabegron significantly increased BC (p<0.05) whereas Cpd (IA) significantly decreased BC (p<0.05, FIG. 5D). When compared to vehicle, expressed in mL, no significant difference in BC was observed in Mirabegron and Cpd (IA) groups (p>0.05). Expressed in % of variation from basal values, Cpd (IA) significantly decreased BC (p<0.05) whereas no significant difference was observed after mirabegron treatment (p>0.05).

When compared to basal values, in Sham and BOO rats, vehicle did not significantly affect ThP (p>0.05). In BOO rats, mirabegron was without effect on ThP (p>0.05) whereas Cpd (IA) significantly decreased ThP (p<0.01). When compared to vehicle, expressed in mmHg, ThP was not significantly modified after mirabegron or Cpd (IA) treatment (p>0.05). Expressed in % variation from basal values, ThP was significantly decreased after mirabegron or Cpd (IA) treatment (p<0.05).

In sham rats, vehicle significantly increased AM (p<0.01) compared to basal values. In BOO rats, vehicle did not significantly affect AM (p>0.05) whereas mirabegron and Cpd (IA) did not significantly modify AM (p>0.05).When compared to vehicle, expressed in mmHg or in % variation from basal values, no effect on AM was observed after mirabegron or Cpd (IA) treatment (p>0.05).

In sham and BOO rats, no significant effect on NVC frequency was observed after vehicle (p>0.05) compared to basal values. In BOO rats, mirabegron significantly decreased NVC frequency (p<0.01) and Cpd (IA) did not affect NVC frequency (p>0.05). When expressed to vehicle, expressed in number of NVC/min or in % variation from basal values, NVC frequency was unaffected after mirabegron or Cpd (IA) treatment (p>0.05).

Vehicle did not significantly modified NVC amplitude (p>0.05) in sham and BOO rats compared to basal values. In BOO rats, mirabegron significantly reduced NVC amplitude (p<0.05), whereas Cpd (IA) was without any effect on NVC amplitude. When compared to vehicle, expressed in mmHg, NVC amplitude was not significantly modified after mirabegron or Cpd (IA) treatment (p>0.05). Expressed in % variation from basal values, NVC amplitude was not significantly modified after Cpd (IA) treatment whereas mirabegron significantly decreased NVC amplitude (p>0.05 and p<0.05).

### Results of Intravenous (i.v.) Administration

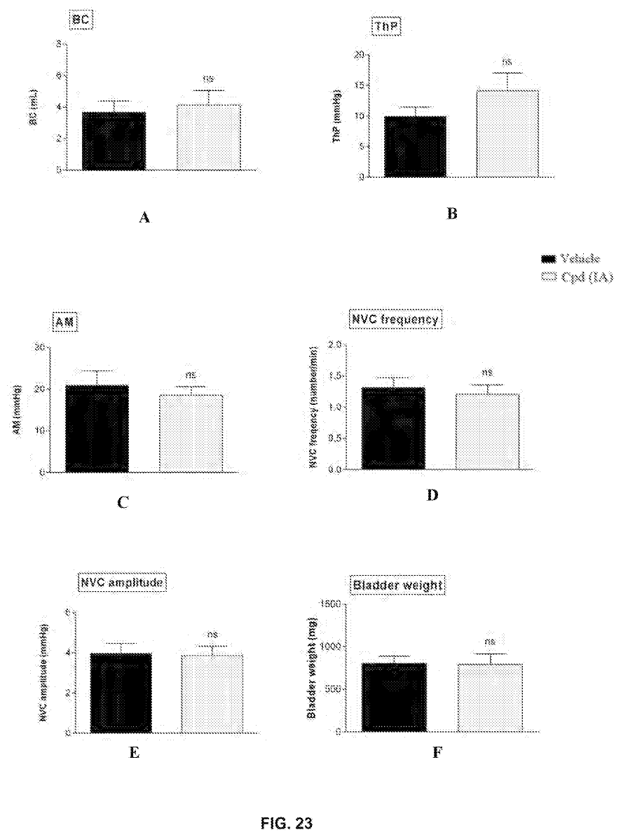

The basal values of BC, ThP, AM, NVC frequency, NVC amplitude and bladder weight were not significantly different in both experimental BOO groups (p>0.05, FIG. 23), including vehicle (1 mL/kg, i.v.), and Cpd (IA) (3 mg/kg, i.v.) groups.

Results from the 0-90 min entire period:

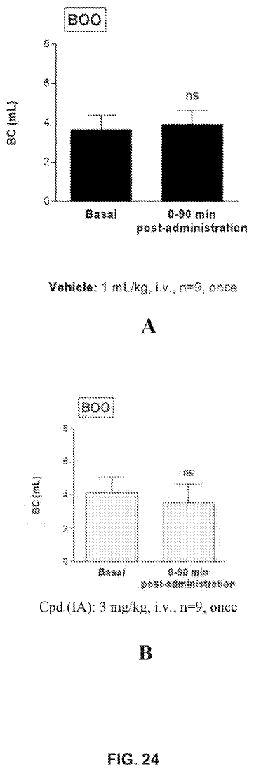

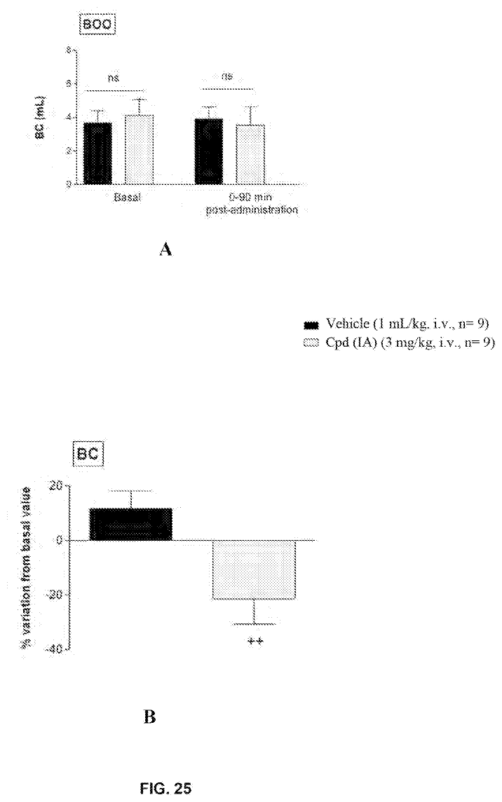

When compared to basal values, in BOO rats, vehicle and Cpd (IA) did not significantly modify BC (p>0.05, FIGS. 24A and 24B). When compared to vehicle, expressed in mL, no significant difference in BC was observed in Cpd (IA) group (p>0.05, FIG. 25A). Expressed in % of variation from basal values, Cpd (IA) significantly decreased BC (p<0.01, FIG. 25B).

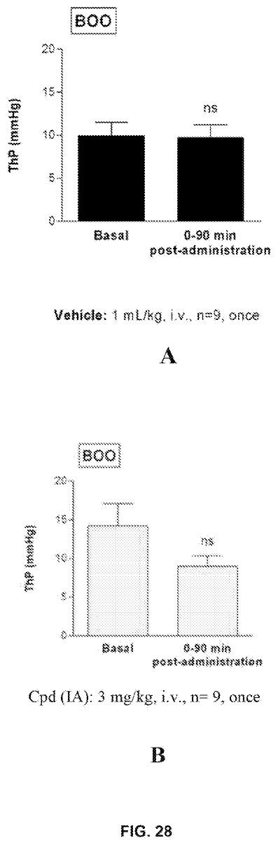

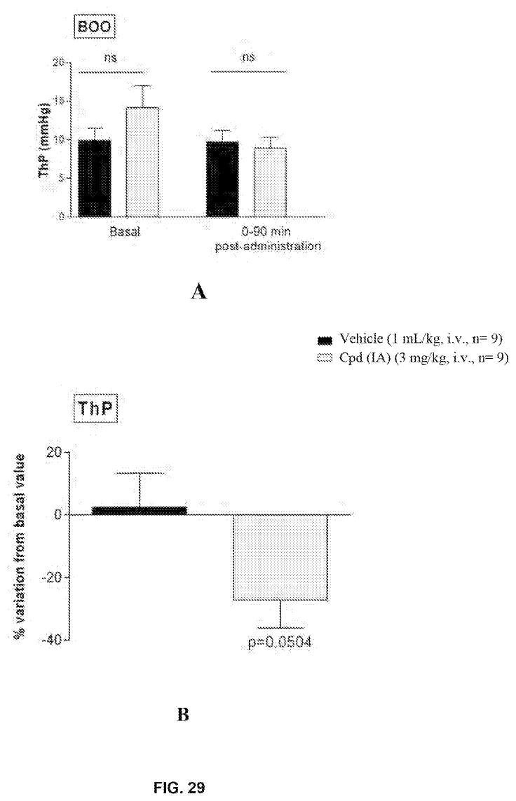

When compared to basal values, no significant effect on ThP was observed after vehicle and Cpd (IA) treatments (p>0.05, FIGS. 28A and 28B). Compared to vehicle, expressed in mmHg, ThP was not significantly modified after Cpd (IA) treatment (p>0.05, FIG. 29A). Expressed in % variation from basal values, ThP was decreased after Cpd (IA) treatment, the p value was close to the significance (p=0.0504, FIG. 29B).

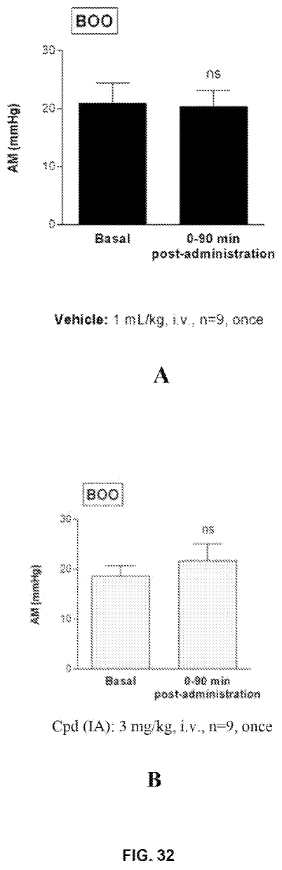

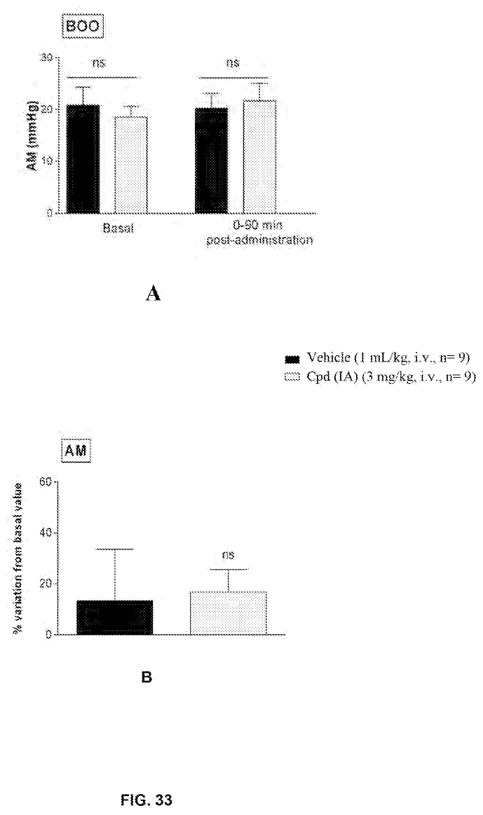

When compared to basal values, in BOO rats, vehicle and Cpd (IA) did not significantly modify AM (p>0.05, FIGS. 32A and 32B). Compared to vehicle, expressed in mmHg or in % variation from basal values, AM was not significantly modified after Cpd (IA) treatment (p>0.05, FIGS. 33A and 33B).

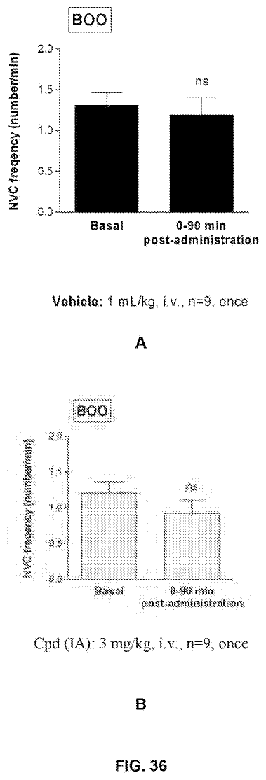

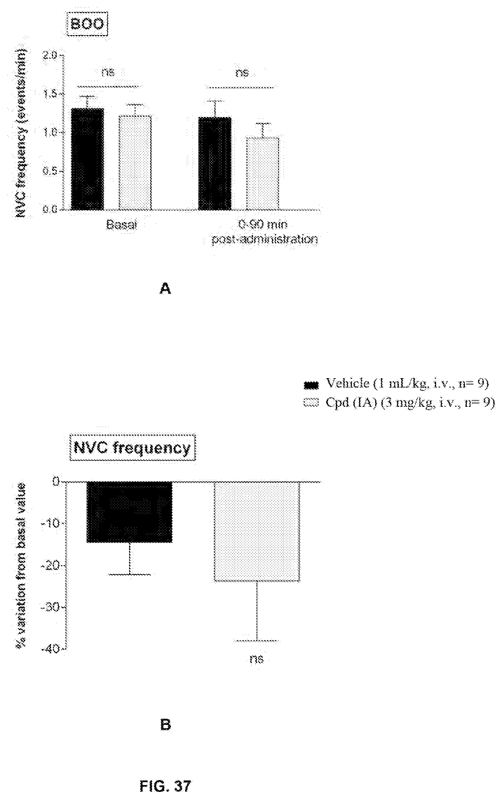

When compared to basal values, in BOO rats, vehicle and Cpd (IA) did not affect NVC frequency (p>0.05, FIGS. 36A and 36B). Compared to vehicle, expressed in number of NVC/min or in % variation from basal values, NVC frequency was not significantly modified after Cpd (IA) treatment (p>0.05, FIGS. 37A and 37B).

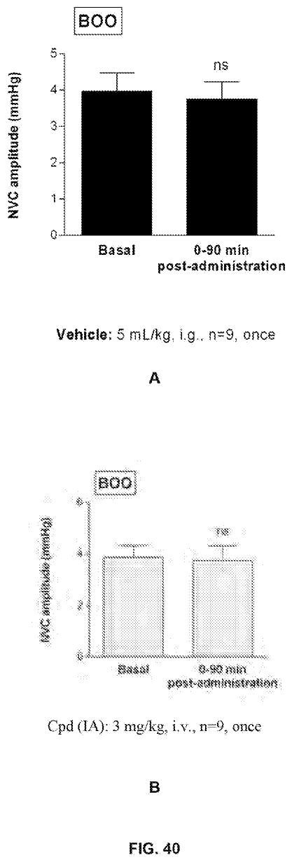

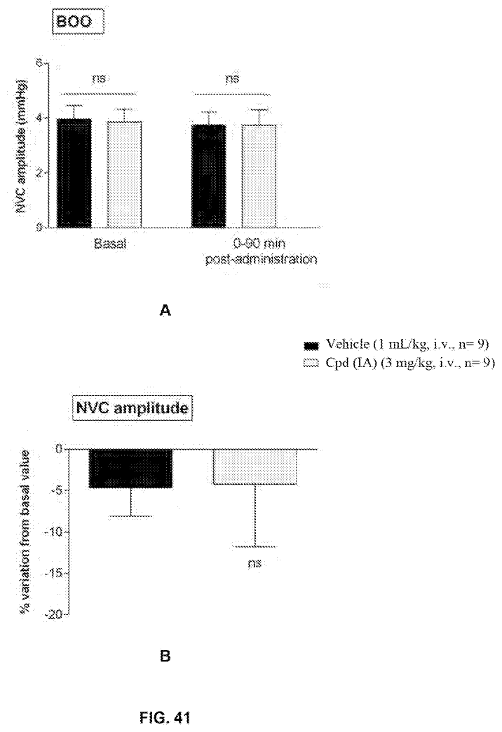

When compared to basal values, vehicle and Cpd (IA) did not modify NVC amplitude (p>0.05, FIGS. 40A and 40B). Compared to vehicle, expressed in number of mmHg and in % variation from basal values, NVC amplitude was not significantly modified after Cpd (IA) treatment (p>0.05, FIGS. 41A and 41B).

Results from the 60-90 min interval period:

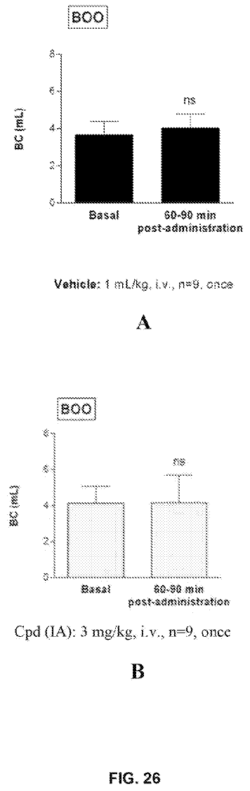

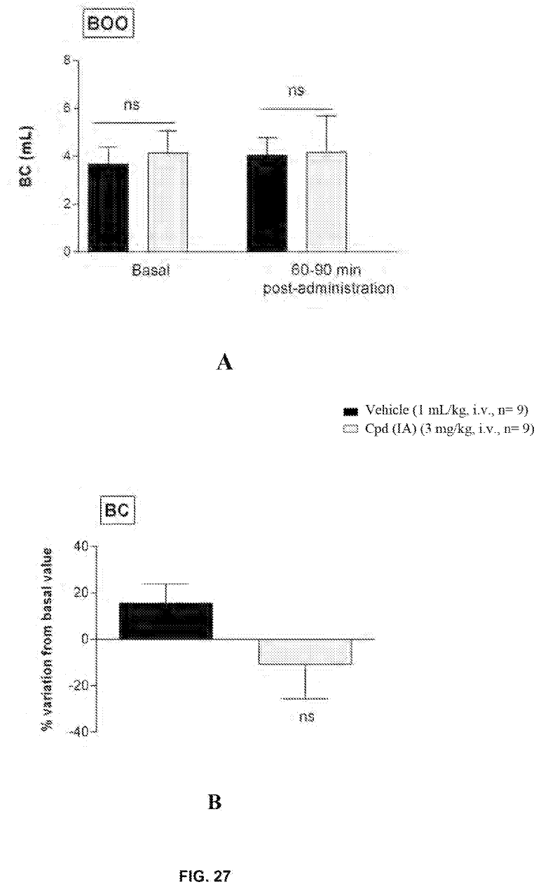

When compared to basal values, In BOO rats, neither vehicle nor Cpd (IA) had a significant effect on BC (p>0.05, FIGS. 26A and 26B). When compared to vehicle, expressed in mL and in % of variation from basal values, no significant difference in BC was observed in Cpd (IA) group (p>0.05, FIGS. 27A and 27B).

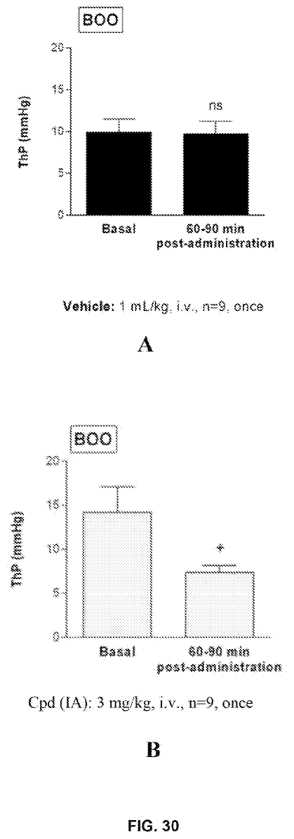

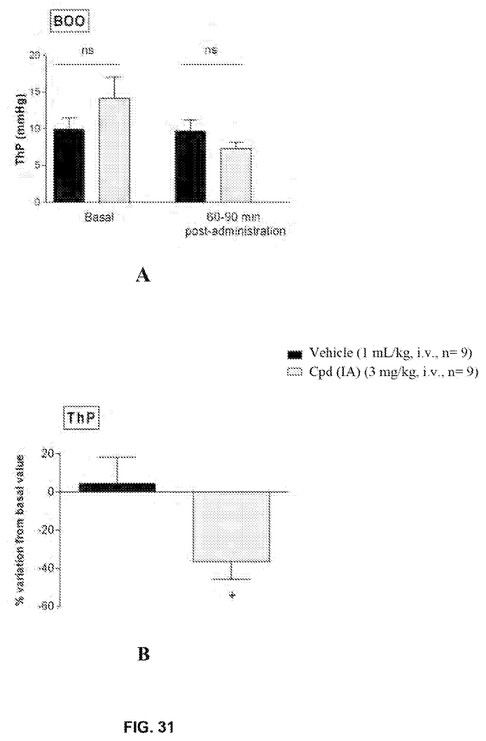

When compared to basal values, in BOO rats, vehicle did not significantly affect ThP (p>0.05, FIG. 30A). In contrast, Cpd (IA) significantly decreased ThP (p<0.05, FIG. 30B). Compared to vehicle, expressed in mmHg, ThP was not significantly modified after Cpd (IA) treatment (p>0.05, FIG. 31A). Expressed in % variation from basal values, ThP was significantly decreased after Cpd (IA) treatment (p<0.05, FIG. 31B).

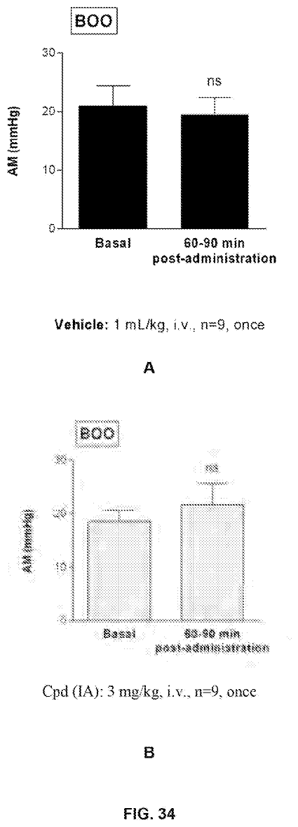

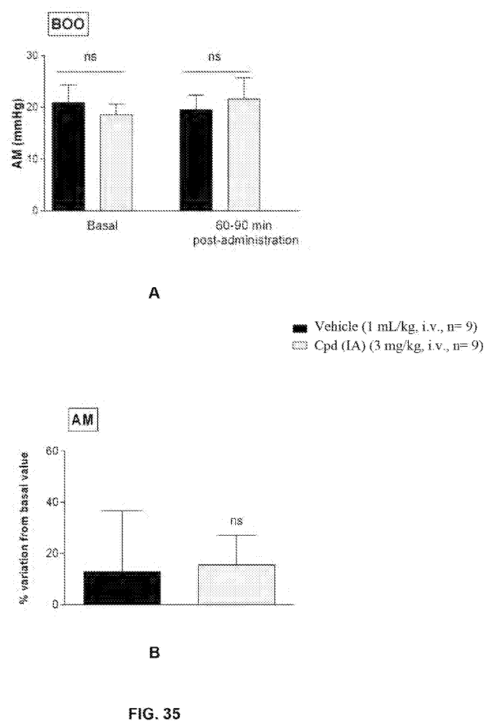

When compared to basal values, in BOO rats, vehicle and Cpd (IA) did not significantly modify AM (p>0.05, FIGS. 34A and 34B). Compared to vehicle, expressed in mmHg or in % variation from basal values, no effect on AM was observed after Cpd (IA) treatment (p>0.05, FIGS. 35A and 35B).

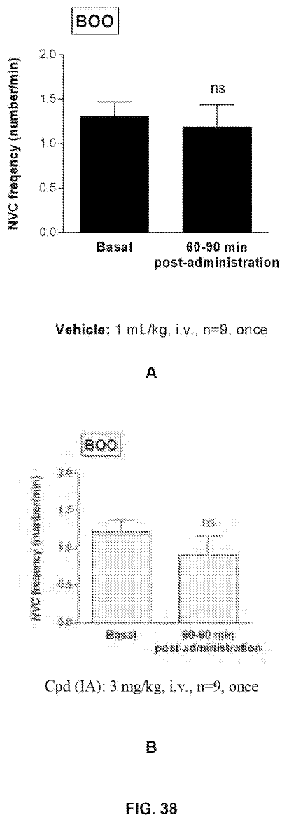

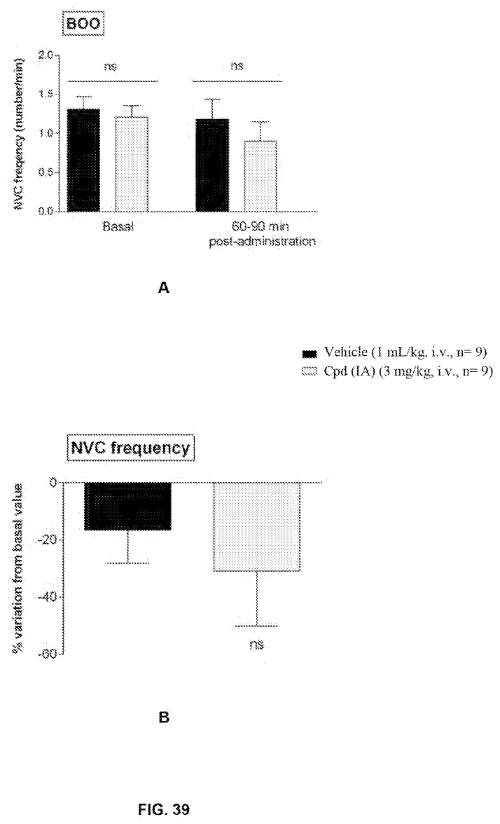

When compared to basal values, in BOO rats, no significant effect on NVC frequency was observed after vehicle and Cpd (IA) (p>0.05, FIGS. 38A and 38B). Compared to vehicle, expressed in number of NVC/min or in % variation from basal values, NVC frequency was unaffected after Cpd (IA) treatment (p>0.05, FIGS. 39A and 39B).

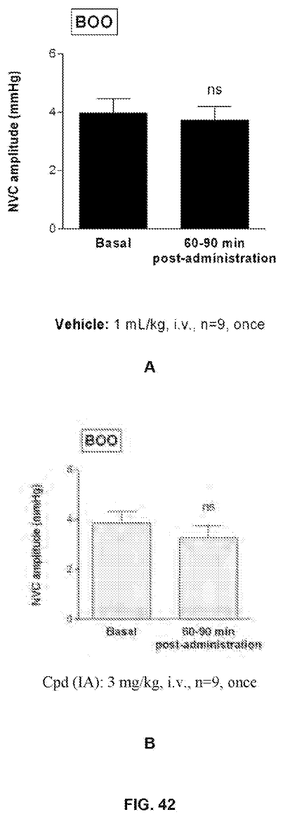

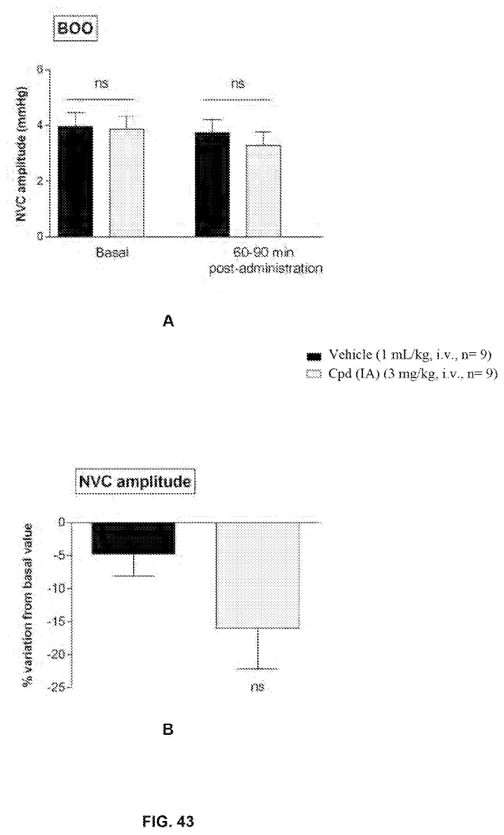

When compared to basal values, in BOO rats, vehicle and Cpd (IA) were without any effect on NVC amplitude (p>0.05, FIGS. 42A and 42B). Compared to vehicle, expressed in mmHg and in % variation from basal values, NVC amplitude was not significantly modified after Cpd (IA) treatment (p>0.05, FIGS. 43A and 43B).

### Conclusions

In sham rats, compared to basal, vehicle (5 mL/kg, i.g.) increased BC just for the 60-90 min interval period. These effects on BC after administration were probably due to a time effect. Vehicle also significantly increased AM for the entire period (90 min) and for the last interval period (60-90 min) compared to basal values. In BOO rats, vehicle (5 mL/kg, i.g.) had no effect on all cystometric parameters allowing confounding factors for the evaluation of test substances.

As expected, mirabegron (10 mg/kg, i.g.) significantly increased BC compared to basal values and decreased NVC amplitude and frequency compared to vehicle and basal values, respectively (Gillespy et al., 2012).

As observed in SCI model study, Cpd (IA) (i.g.) significantly decreased BC and ThP compared to vehicle. The effect observed on ThP could explain the decrease in bladder capacity since micturition occurs at lower intravesical pressure. The significant decrease in BC after the treatment was similar to the results obtained in SCI rats.

In BOO rats, vehicle (1 mL/kg, i.v.) had no effect on all cystometric parameters allowing confounding factors for the evaluation of test substances. As observed in SCI study and in BOO rats with intra-gastric administration, Cpd (IA) (i.v.) significantly decreased BC and ThP compared to vehicle and had no effect on all other cystometric parameters.

These results suggest that the difference of effects observed in SCI and BOO rats compared to normal rats (isovolumetric model) is probably due to a different involvement of afferent fibers due to a remodeling in pathologic models (SCI and BOO rats) (De Groat, 1995, De Groat & Yoshimura, 2010 and Aizawa et al., 2017).

### Example 5: Phase 1b, Blinded, Placebo-Controlled Crossover Study Assessing the Effects of Oral Administration of the Compound of Formula (IA) in Female Human Subjects with Overactive Bladder

The study includes a Screening/Washout Period (up to 4 weeks), Single-blind Placebo Run-in Period (2 weeks), Double-blind Treatment Period (8 weeks), Single-blind Placebo Washout Period (1 week), and Follow-up Period (up to 1 week). The study design is summarized in Table 8:

TABLE 8

| | Pre-randomization | | | Post-randomization | | | | | | | | | |
|---|---|---|---|---|---|---|---|---|---|---|---|---|---|
| Phase | Screening/ | Single-blind | | | | | | | | | | Single-blind | Follow- |
| Period | washout | Run-in | | Double-blind Treatment | | | | | | | | washout | up |
| Day | −42 to −16 | −15 | −1^{A} | 1 | 14 | 15 | 28 | 35 | 42 | 49 | 56 | 63 | 70 |
| Clinic Visit | V1 | V2 | V3^{A} | | V4 | | V5 | | V6 | | V7 | V8 | V9 |
| Phone call ^{B} | | | | P1 | | P2 | | P3 | | P4 | | | |

| | | | | |
|---|---|---|---|---|
| Tx Sequence A | Placebo | Placebo | Compound of Formula (IA) | Placebo |

| | | | | |
|---|---|---|---|---|
| Tx Sequence B | Placebo | Compound of Formula (IA) | Placebo | Placebo |

^{A}Visit 3, Day −1, is end of Single-blind Run-in Period and start of the Double-blind Treatment Period. At visit 3, a subject that has met all inclusion/exclusion criteria, has met all randomization eligibility criteria, and has completed all study procedures, will be dispensed double-blind study-drug with instruction to ingest first dose that evening at bedtime.
^{B} Phone contact between clinic visits to assess tolerability and review diary/dosing instructions.

Screening/Washout Period: Informed consent is obtained from each subject before any study procedures are performed in this study. The assessment of study eligibility criteria is initiated at the Screening visit (Visit 1) and includes medical history, physical examination, vital signs, clinical laboratory tests, urine culture, pregnancy test, and drug screen. If a washout of prohibited medications is required, this washout is completed during the Screening/Washout period.

The subject stops current medication treatment for OAB while continuing any ongoing non-clinical treatments in a similar and consistent manner throughout study (which can include timed voiding and behavioral modification therapy, dietary restrictions, and stress reduction).

There are no minimum number of days of screening and a subject can be entered into the Run-In Period as soon as they satisfy the eligibility criteria.

Single-blind Run-in Period: Eligible subjects will be entered into the Single-blind Run-in Period (Visit 2). The subject must have overactive bladder symptoms after wash-out of previous overactive bladder medications. During the Run-In Period, subjects are instructed to ingest 1 tablet of study drug each evening at bedtime.

The subject records their individual symptomology related to OAB using the Micturition Diary and other assessments. The subject completes a the Symptom Impact Sleep Questionnaire (SISQ) in the morning.

During the week that preceded the next clinic visit (Visit 3), the subject records the micturition time, type, and urgency for each episode for a minimum of 3 and up to 7-days.

At Visit 3, the subject is evaluated for eligibility to be randomized. The eligibility is confirmed to the site prior to randomization through an algorithm taking into account micturition components recorded during the run-in period. In addition, the PI confirms that the subject does not take any medication (other than study drug) for treatment of OAB symptoms during the run-in, and that all diaries are completed appropriately, according to protocol.

Visit 3, Day-1, is the end of Single-blind Run-in Period and start of the Double-blind Treatment Period.

Double-blind Treatment Period: At visit 3, a subject who continues to meet eligibility criteria and meets all randomization eligibility criteria is randomized into the study and dispensed double-blind study drug with instruction to ingest first dose that evening at bedtime. The subject attends clinic visits every 2 weeks, and phone contact is required on day 1 and 15 and at least once during each week between clinic visits to assess tolerability and review diary/dosing instructions.

During the week that preceded each clinic visit (Visit 4, Visit 5, Visit 6 and Visit 7 and Visit 8), the subject records the micturition time, type, and urgency for each episode for a minimum of 3 and up to 7-days.

At clinic visits, the subject completes the efficacy/safety and other assessments.

Single-blind Washout Period: The subject follows the assessments. During the week that preceded clinic visit (Visit 8), the subject records the micturition time, type, and urgency for each episode for a minimum of 3 and up to 7-days.

End of Treatment (EOT)/Early Termination (ET): Subjects undergo EOT procedures at either the end of Double-blind Treatment, or upon early discontinuation from the study. If assessments call for at EOT were performed on the same day of study completion or on the same day as ET and no study drug is administered after those assessments, those assessments are not repeated.

Follow-up Period/End of Study (EOS): A follow-up phone call is completed approximately one week after the last dose of study drug to monitor AEs and concomitant medication/therapy since the previous visit.

Inclusion criteria:
1. Be female, age ≥18 years and capable of voiding independently. A female is eligible to participate if she is not pregnant, not breastfeeding, and at least one of the following applies: a female is considered to be of childbearing potential unless she has had a hysterectomy, has undergone tubal ligation or is at least one year post-menopausal; or female participants of childbearing potential must agree to use a reliable method of contraception with a failure rate of less than 1% per year when used consistently and correctly such as implants, injectables, combined oral contraceptives, some intra uterine devices [IUDs], sexual abstinence or vasectomized partner during the trial and for 30 days following the end of study
2. Have symptoms of overactive bladder including urinary urgency and urinary frequency with or without incontinence for ≥3 months.
3. Be willing to stop current medication treatment for overactive bladder (including anticholinergics and beta-3 antagonists) and other prohibited medications (including antipsychotics, opioids, GABA receptor analogues/modulators/agonists, and renal transport inhibitors) while continuing any ongoing non-clinical treatments in a similar and consistent manner throughout study (which may include timed voiding and behavioral modification therapy, dietary restrictions, and stress reduction).
4 Be considered, in the opinion of the clinical site investigator, to be generally healthy based on the results of a medical history, physical examination, 12-lead ECG, and laboratory profile
5. Be able to understand the consent form and effectively communicate with the study staff.
6. Voluntarily provide written informed consent and be willing and able to complete all study procedures including electronic diaries and study related questionnaires.
   Exclusion criteria:
1. Had UTI, including bacterial cystitis, within the past 30 days, or has a history of recurrent UTI as defined as 3 or more culture documented episodes in the last 6 months.
2. Hematuria determined to be associated with bladder malignancy or other significant pathology.
3. Had surgical procedure at any time that affected bladder function, bladder incontinence surgery, urethral surgery, or received a botulinum toxin bladder injection within the past 6 months.
4. Has started using a sacral and/or pudendal nerve neuromodulation device (e.g. Interstim) in the last 9 months. Subjects with history of percutaneous nerve stimulation for OAB are eligible if the device was started >9 months ago and the subject has been on a stable setting for the past 6 months, and approval of medical monitor.
5. Has clinically significant outflow obstruction (at the discretion of the investigator).
6. Has an indwelling catheter or intermittent self-catheterization.
7. Has a neurological cause for abnormal detrusor activity and/or diabetic neuropathy, and/or has uncontrolled diabetes. Subjects with early stage or controlled diabetes are eligible on basis of A1C records and approval of medical monitor.
8 Has chronic inflammation (e.g., interstitial cystitis) bladder stones, previous pelvic radiation therapy or previous/current malignant disease of the pelvic organs (within confines of pelvis). Had cyclophosphamide or chemical cystitis, or tuberculosis, or pelvic radiation or active genital herpes or vaginitis.
9. Has Grade III/IV pelvic organ prolapse with/without cystocele or urethral diverticulum.
10. Has moderate to severe hepatic impairment defined as Child-Pugh Class B or C.
11. Has any history and/or current evidence of other medical (e.g., cardiac, respiratory, gastrointestinal, renal, malignancy other than basal cell carcinoma), neurological, or psychiatric conditions that, in the opinion of the investigator, could affect the subject's safety or interfere with the study assessments or might interfere with drug absorption, distribution, metabolism, or excretion (including any surgical interventions for weight loss).
12. Has positive findings on the Columbia-Suicide Severity Rating Scale (C-SSRS) or current or history of suicidal ideation/behavior. Positive findings on C-SSRS include but, are not limited to, the following: suicidal ideation associated with actual intent and a method or plan in the past year (“Yes” answer on items 4 or 5 of the C-SSRS); a previous history of suicidal behaviors in the past 5 years (“Yes” answer to any of the suicidal behavior items of the C-SSRS); or any lifetime history of serious or recurrent suicidal behavior.
13. Has current or recent history (within past 6 months) of drug abuse or dependence, or a diagnosis of substance use disorder.
14. Has current or history of clinically significant kidney disease or abnormal kidney function, or nephrolithiasis (findings or symptoms in last 3 years) or estimated glomerular filtration rate (eGFR)<60 mL/min/1.73 m2
15. Was dosed in a clinical drug study during the 30 days prior to the screening visit.

### Randomization Criteria

At Visit 3, subjects meet the following randomization criteria to be eligible for entry into the Double-blind Treatment Period: on at least 3 days in Void Diary, subject must have had (i) 8 or more voids per day (24 hours); and (ii) 1 or more voids per day (24 hours) with urgency grade ≥1.

Study treatments:

Details of the treatments administered are provided in Table 9.

TABLE 9
Treatments Administered

| | Compound of | |
|---|---|---|
| Study Treatment Name | Formula (IA) | Placebo |
| Dosage formulation | Tablet | Tablet |
| Unit dose strength(s) | 1 mg dose | NA |
| Route of Administration | Oral | Oral |
| Dosing instructions | Ingest 1 tablet daily | Ingest 1 tablet daily |
| | at bedtime (qhs) | at bedtime (qhs) |

Lifestyle considerations:

Sleep/wake pattern: Subjects are encouraged to continue a consistent sleep/wake pattern during the period of the study.

Meals and Dietary Restrictions: During the study, the subjects are instructed to keep a regular daily meal schedule and to not make changes to their usual diet (e.g., beginning a weight loss program).

Caffeine, Alcohol and Tobacco: Enrolled subjects are instructed to limit their caffeine and alcohol consumption. Subjects who are smokers will be encouraged not to vary the number of cigarettes they smoke per day during study participation.

Concomitant/Prohibited medications:

During screening/washout period, subject stopped all current medication treatment for OAB while continuing any ongoing behavioral therapies in similar/consistent manner throughout the study. Any OAB medication is discontinued at least 7 days prior to starting the Single-blind Run-in Period.

Any prohibited medications are discontinued at least 7 days prior to starting the Single-blind Run-in Period. If a medication does not fit into a class of medications noted, the Medical Monitor is consulted to determine whether it is permitted or not.

### Study Endpoints:

A list of study objectives and endpoints are described in Table 10.

TABLE 10
Objectives and Endpoints

| Objectives | Endpoints |
|---|---|
| Primary | |
| Assess effects of | Micturition time, type, and urgency rating will be recorded |
| Compound of Formula | for all episodes during the week preceding each scheduled |
| (IA) on micturition | investigative site clinic visit (minimum of 3 days and up to 7- |
| episode components in | days collection). |
| subjects with overactive | Episode time will be recorded using standard 12 hour |
| bladder syndrome | with am/pm |
| | Episode type will be categorized as either urinated |
| | (passed urine in toilet) or incontinence (involuntary |
| | release of urine). |
| | Episode urgency, defined as the sensation of a sudden, |
| | compelling desire to urinate that is difficult to defer, will |
| | be rated for each episode according to the Patient |
| | Perception of Intensity of Urgency Scale (PPIUS) that |
| | uses a 5-point categorical scale (0 = no urgency, 1-mild |
| | urgency, 2 = moderate urgency, 3 = severe urgency, and |
| | 4 = urge incontinence). |
| Secondary | |
| Assess effects of | Patient Perception of Bladder Condition (PPBC) |
| Compound of Formula | Patient Perception of Treatment Benefit Questionnaire |
| (IA) on other symptom | (OAB-q) |
| components of overactive | Subject Global Response Assessment (SGRA) |
| bladder syndrome. | Patient Perception of Intensity of Urgency Scale (PPIUS) |
| | Symptom Impact Sleep Questionnaire (SISQ) |
| Safety | |
| Assess safety and | Adverse events, Clinical laboratory values, |
| tolerability of Compound | Electrocardiograms, Physical examination, Vital signs, |
| of Formula (IA). | Columbia-Suicide Severity Rating Scale (C-SSRS), Epworth |
| | Sleepiness Scale (ESS) |

### Example 6: Phase 1, Nonrandomized, Open-Label, Parallel-Group Study to Assess the Effect of Mild Renal Impairment on the Single-Dose Pharmacokinetics and Safety of the Compound of Formula (IA)

### Study Design (Methodology):

This was a phase 1, nonrandomized, open-label, parallel-group, single-dose study. A total of 20 subjects (10 subjects with mild renal impairment and 10 healthy subjects with normal renal function) were studied.

At screening, subjects were assigned to a study group according to estimated glomerular filtration rate (eGFR), calculated using the 2021 Chronic Kidney Disease Collaboration (CKD-EPI 2021) formula as follows, in Table 11:

TABLE 11
Study Groups

| | Renal | eGFR |
|---|---|---|
| Group | Function | [(mL/min)/1.73 m²] |
| A | Normal (control) | ≥90 |
| B | Mild impairment | 60 to 89 |

Abbreviation: eGFR, estimated glomerular filtration rate.

Healthy subjects (group A) were matched to the mild renally impaired subjects (group B) according to gender distribution, mean age (±10 years), mean body weight (±15%) and mean body mass index (BMI; ±20%).

All subjects were screened within 28 days before check-in, entered the clinical site the day before study drug dosing (day-1), and received a single oral dose of the compound of formula (IA) on day 1.

A single oral dose of the compound of formula (IA) in an 1.0-mg immediate-release tablet was administered with 240 mL of water in the morning following an overnight fast of at least 10 hours. Subjects remained fasted for 4 hours after study drug dosing (except diabetic subjects, who were allowed a light breakfast at 2 hours after study drug dosing as judged by the investigator). Except as part of study drug administration, subjects restricted their consumption of water for 2 hours before dosing and for 2 hours after dosing. At all other times during the study, subjects were able to consume water ad libitum.

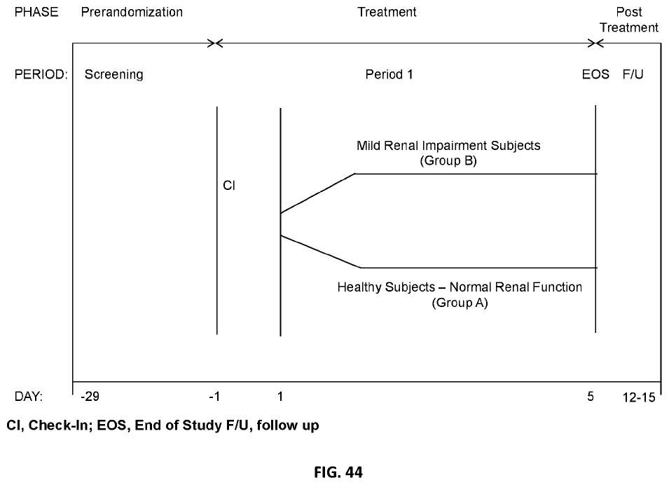

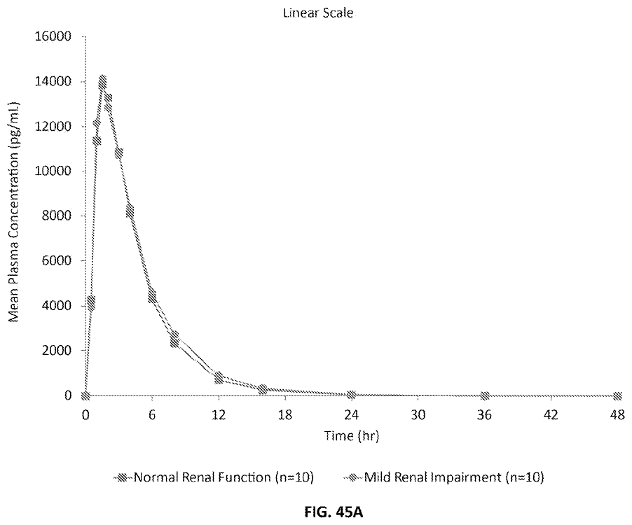

Subjects had end-of-study (EOS) procedures and were discharged from the clinical site on day 5 (or upon early discontinuation). Subjects received a follow-up telephone call 7 to 10 days after EOS. FIG. 44 shows a diagram of the study design.

### Criteria for Inclusion/Exclusion:

Male and female subjects aged 18 to 70 years, inclusive, considered suitable by the investigator to take part in this clinical study and with no clinically significant medical history, except (for subjects with renal impairment only) as associated with their renal impairment or other stable concomitant medical condition.

### Drug Concentration Measurements:

For all subjects, blood samples were collected for the analysis of concentrations of the compound of Formula (IA) before dosing and through 96 hours after dosing. Blood samples collected predose and at 0.5, 1, 1.5, 2, 3, 4, 6, 8, 12, 16, 24, 36, 48, 72 and 96 hours after dosing for determination of plasma concentrations of the compound of Formula (IA).

### Pharmacokinetic Results:

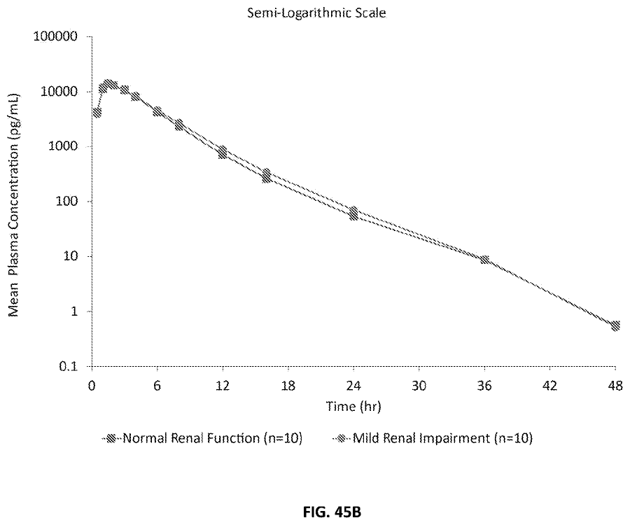

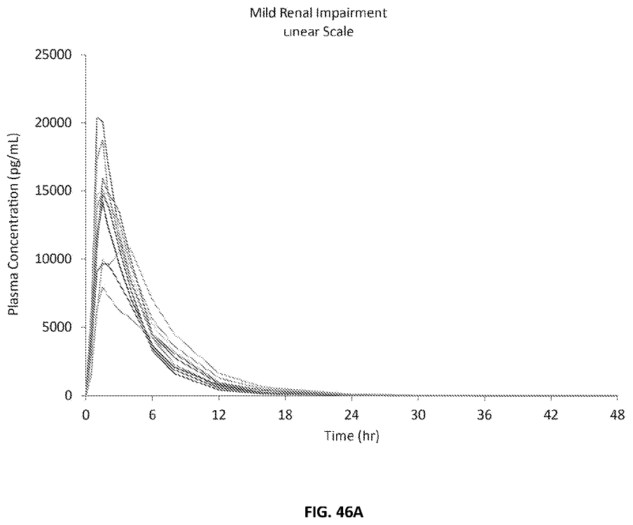

For all subjects, plasma concentrations at 72 and 96 hours were all below the limit of quantitation (concentration-0 pg/mL), therefore, FIGS. 45-47 describe plasma concentrations from 0 through 48 hours post treatment administration.

FIG. 45A describes mean plasma concentrations versus time profiles on a linear scale for subjects with normal renal function (n=10) and subjects with mild renal impairment (n=10). FIG. 45B describes mean plasma concentrations versus time profiles on a semi-logarithmic scale for subjects with normal renal function (n=10) and subjects with mild renal impairment (n=10).

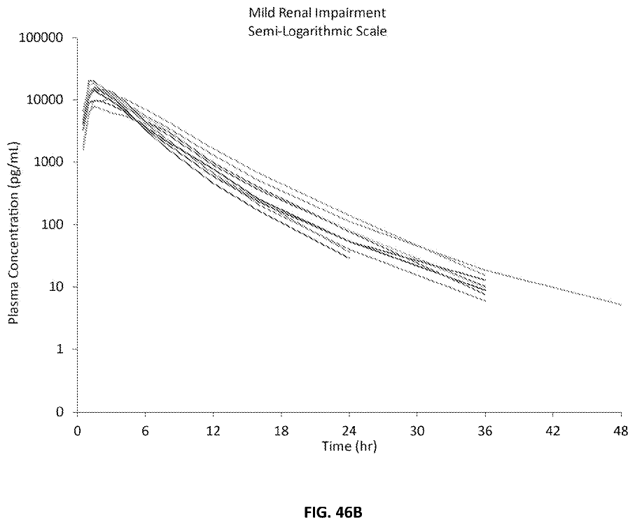

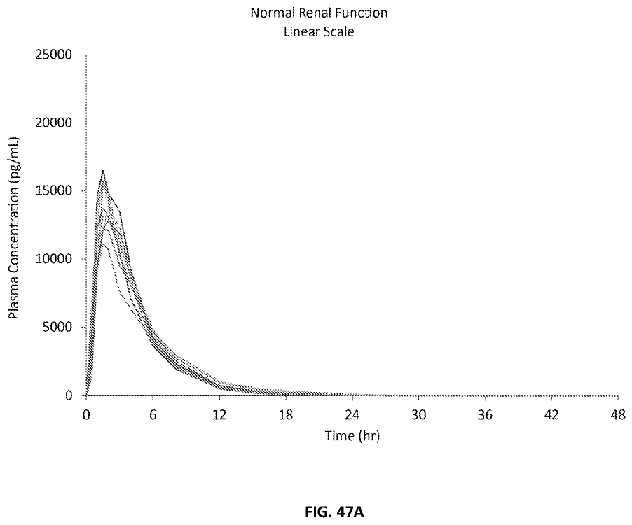

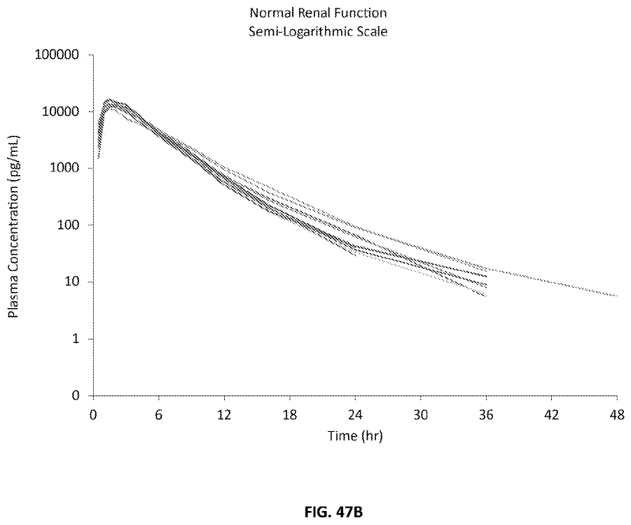

FIG. 46A describes plasma concentrations versus time profiles on a linear scale for individual subjects with mild renal impairment (n=10). FIG. 46B describes plasma concentrations versus time profiles on a semi-logarithmic scale for individual subjects with mild renal impairment (n=10). FIG. 47A describes plasma concentrations versus time profiles on a linear scale for individual subjects with normal renal function (n=10). FIG. 47B describes plasma concentrations versus time profiles on a semi-logarithmic scale for individual subjects with normal renal function (n=10).

As shown in FIGS. 45-47, following a single 1.0-mg immediate-release tablet of the compound of formula (IA), plasma concentrations over time in subjects with mild renal impairment were comparable to those in subjects with normal renal function.

While the subject matter of this disclosure has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

### REFERENCES

FDA 2019 Draft Guidance Document. Interstitial Cystitis Bladder Pain Syndrome (IC/BPS): Establishing Effectiveness of Drugs for Treatment Guidance for Industry.
Bosch P C. Examination of the Significant Placebo Effect in the Treatment of Interstitial Cystitis/Bladder Pain Syndrome. Urology 2014; 84 (2): 321-325
Beauval J B et al., 2015. Comparison of the effects of β3-adrenoceptor agonism on urinary bladder function in conscious, anesthetized, and spinal cord injured rats. Neurourol Urodyn. 34:578-585.
Cheng C et al., 1995. Effect of capsaicin on micturition and associated reflexes in chronic spinal rats. Brain Research 678:40-48.
De Groat W C, 1995. Mechanism underlying the recovery of lower urinary tract function following spinal cord injury. Paraplegia 33:413-501.
De Groat W C & Yoshimura N., 2010. Changes in afferent activity after spinal cord injury. Neurourol Urodyn. 29:63-76.
Kadekawa K et al., 2017. Effects of an alpha1A/D-adrenoceptor antagonist, naftopidil, and a phosphodiesterase type 5 inhibitor, tadalafil, on urinary bladder remodeling in rats with spinal cord injury. Neurourol Urodyn. 36:1488-1495.
Wada N et al., 2017. Combination effects of muscarinic receptor inhibition and β3-adrenoceptor stimulation on neurogenic bladder dysfunction in rats with spinal cord injury. Neurourol Urodyn. 36:1039-1045.
Wada N et al., 2018. Urodynamic effects of intravenous and intrathecal administration of E-series prostaglandin 1 receptor antagonist on detrusor overactivity in rats with spinal cord injury. Neurourol Urodyn. 37:132-137.
Yoshiyama M et al., 1999. Changes in micturition after spinal cord injury in conscious rats. Urology. 54:29-933.
Aizawa N et al., 2017. Characteristics of the Mechanosensitive Bladder Afferent Activities in Relation With Microcontractions in Male Rats With Bladder Outlet Obstruction. Sci Rep 7:7646.
De Groat W C, 1995. Mechanism underlying the recovery of lower urinary tract function following spinal cord injury. Paraplegia 33:413-501.
De Groat W C & Yoshimura N., 2010. Changes in afferent activity after spinal cord injury. Neurourol Urodyn. 29:63-76.
Gillespie J. et al., 2012 Modulation of non-voiding activity by the muscarinergic antagonist tolterodine and the β(3)-adrenoceptor agonist mirabegron in conscious rats with partial outflow obstruction. BJU International 110:132-142
Lluel P. et al., 1998. Experimental bladder instability following bladder outlet obstruction in the female rat. J Urol. 160:2253-2257.

What is claimed is:

1. A method of treating or preventing overactive bladder syndrome in a human subject in need of such treatment, comprising administering to the human subject a therapeutically effective amount of a compound of formula (I): 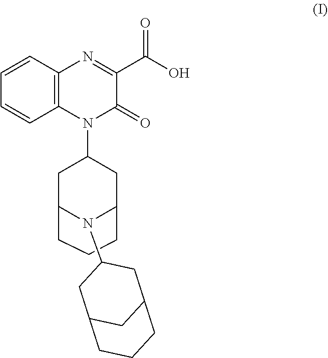 [SMILES: O=C(O)c1nc2ccccc2n(C2CC3CCCC(C2)N3C2CC3CCCC(C3)C2)c1=O]
or a pharmaceutically acceptable salt thereof.

2. The method of claim 1, wherein the compound is a compound of formula (I′): 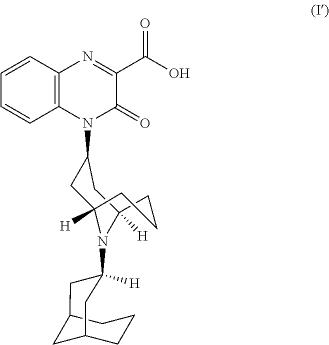
or a pharmaceutically acceptable salt thereof.

3. The method of claim 1 or claim 2, wherein the method comprises administering a pharmaceutically acceptable salt of the compound, wherein the salt is selected from the group consisting of sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, and p-toluenesulfonate salts.

4. The method of any one of claims 1 to 3, wherein the method comprises administering a p-toluenesulfonic acid salt, a sulfate salt, a phosphoric acid salt, or a hydrochloride salt of the compound.

5. The method of any one of claims 1-4, wherein a p-toluenesulfonic acid salt of the compound is administered.

6. The method of any one of claims 1-5, wherein the method comprises administering a compound of formula (IA):

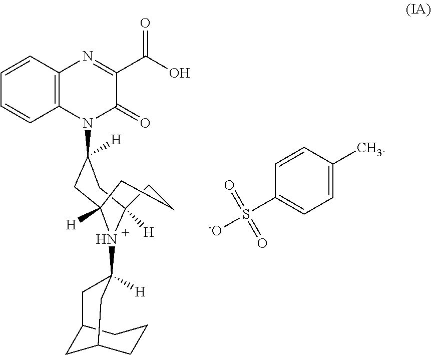

7. The method of any one of claims 1-6, wherein urinary frequency in the human subject is reduced.

8. The method of any one of claims 1-6, wherein episodes of nocturia are reduced in the human subject.

9. A method of reducing nocturia in a human subject in need of such treatment, comprising administering to the human subject a therapeutically effective amount of a compound of formula (I): 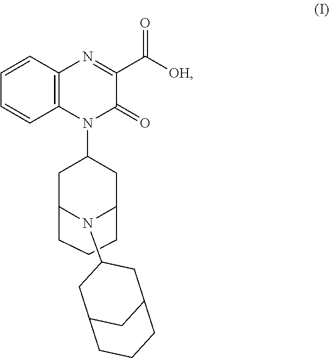 [SMILES: O=C(O)c1nc2ccccc2n(C2CC3CCCC(C2)N3C2CC3CCCC(C3)C2)c1=O]
or a pharmaceutically acceptable salt thereof.

10. The method of claim 9, wherein the compound is a compound of formula (I′): 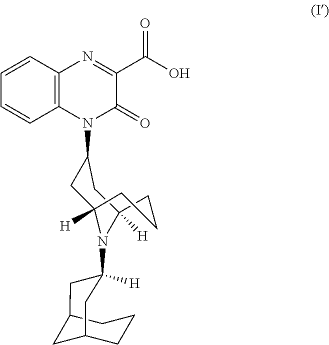
or a pharmaceutically acceptable salt thereof.

11. The method of claim 9 or claim 10, wherein the method comprises administering a pharmaceutically acceptable salt of the compound, wherein the salt is selected from the group consisting of sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, and p-toluenesulfonate salts.

12. The method of any one of claims 9 to 11, wherein the method comprises administering a p-toluenesulfonic acid salt, a sulfate salt, a phosphoric acid salt, or a hydrochloride salt of the compound.

13. The method of any one of claims 9 to 12, wherein a p-toluenesulfonic acid salt of the compound is administered.

14. The method of claim 13, wherein the method comprises administering a compound of formula (IA):

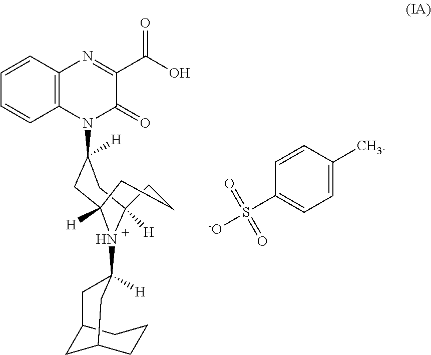

15. The method of any one of claims 1-14, wherein the compound or a pharmaceutically acceptable salt thereof is administered orally, parenterally, intravenously, intramuscularly, buccally, or transdermally.

16. The method of any one of claims 1-15, wherein the compound or a pharmaceutically acceptable salt thereof is administered orally.

17. The method of any one of claims 1-16, wherein the therapeutically effective amount of the compound or a pharmaceutically acceptable salt thereof is from about 0.001 mg to about 300 mg.

18. The method of any one of claims 1-17, wherein the therapeutically effective amount of the compound or a pharmaceutically acceptable salt thereof is from about 0.10 mg to about 10 mg.

19. The method of claim 18, wherein the compound or a pharmaceutically acceptable salt thereof is a compound of formula (IA):

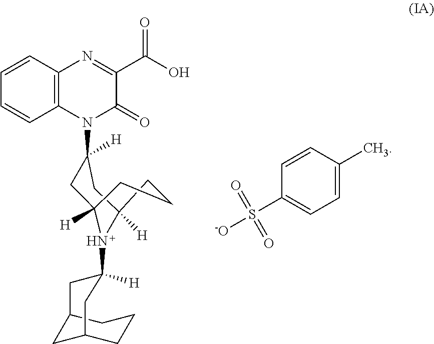

20. The method of any one of claims 1 to 19, wherein the compound or a pharmaceutically acceptable salt thereof is administered once daily.

21. The method of any one of claims 1-20, wherein the compound or a pharmaceutically acceptable salt thereof is administered at night.

22. The method of claim 21, wherein the compound or a pharmaceutically acceptable salt thereof is administered prior to bedtime.

23. The method of any one of claims 16 to 19, wherein the compound or a pharmaceutically acceptable salt thereof is administered twice daily.

24. The method of claim 23, wherein the compound or a pharmaceutically acceptable salt thereof is administered approximately every 12 hours.

25. The method of claim 23 or 24, wherein the method comprises administering a first therapeutically effective amount of the compound or a pharmaceutically acceptable salt thereof during daytime, and administering a second therapeutically effective amount at nighttime prior to bedtime of the human subject.

26. The method of claim 25, wherein the first therapeutically effective amount and the second therapeutically effective amount are the same.

27. The method of claim 25, wherein the first therapeutically effective amount and the second therapeutically effective amount are different.

28. The method of claim 27, wherein the second therapeutically effective amount is about 2-fold greater than the first therapeutically effective amount.

29. The method of claim 27, wherein the second therapeutically effective amount is about 10-fold greater than the first therapeutically effective amount.

30. The method of any one of claims 1-29, wherein the administration of the compound or a pharmaceutically acceptable salt thereof increases a micturition pressure threshold in the human subject by about 30% to 80%.

31. The method of any one of claims 1-30, further comprising administering an effective amount of an antimuscarinic agent to the human subject.

32. The method of claim 31, wherein the antimuscarinic agent is oxybutynin, tolterodine, trospium, solifenacin and darifenacin, or a pharmaceutically acceptable salt of any of the foregoing.

33. A method of treating or preventing overactive bladder syndrome in a human subject in need of such treatment, comprising administering to the human subject a therapeutically effective amount of a compound of formula (I): 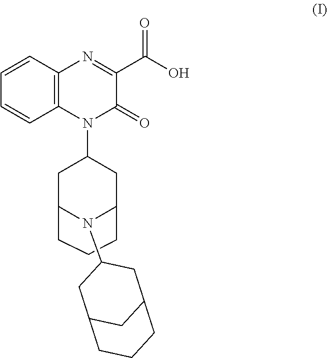 [SMILES: O=C(O)c1nc2ccccc2n(C2CC3CCCC(C2)N3C2CC3CCCC(C3)C2)c1=O]
or a pharmaceutically acceptable salt thereof, wherein the patient additionally suffers from a sleep disorder.

34. The method of claim 33, wherein the compound is a compound of formula (I′): 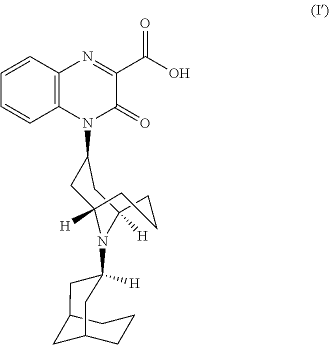
or a pharmaceutically acceptable salt thereof.

35. The method of claim 33 or 34, wherein the method comprises administering a pharmaceutically acceptable salt of the compound, wherein the salt is selected from the group consisting of sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, and p-toluenesulfonate salts.

36. The method of any one of claims 33-35, wherein the pharmaceutically acceptable salt is a p-toluenesulfonic acid salt, a sulfate salt, a phosphoric acid salt, or a hydrochloride salt.

37. The method of any one of claims 33-36, wherein the pharmaceutically acceptable salt a p-toluenesulfonic acid salt.

38. The method of any one of claims 33-37, wherein method comprises administering the compound of formula (IA):

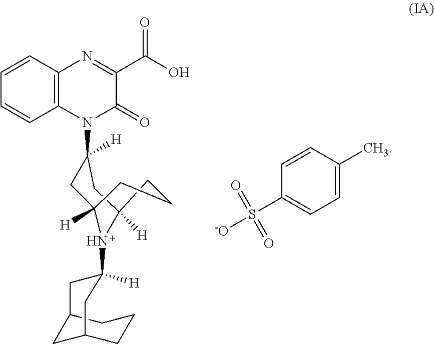

39. The method of any one of claims 33-38, wherein the human subject is a female of 50 years of age or older.

40. A method of treating a sleep disorder in a patient, comprising administering to the patient a therapeutically effective amount of a compound of formula (I): 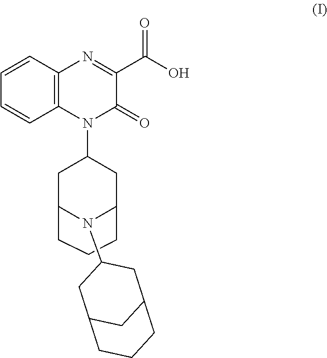 [SMILES: O=C(O)c1nc2ccccc2n(C2CC3CCCC(C2)N3C2CC3CCCC(C3)C2)c1=O]
or a pharmaceutically acceptable salt thereof, wherein said patient also suffers urinary incontinence.

41. The method of claim 40, wherein the compound is a compound of the formula (I′): 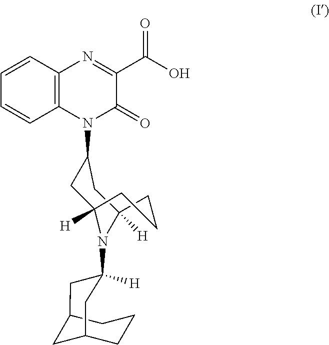
or a pharmaceutically acceptable salt thereof.

42. The method of claim 40 or 41, wherein the method comprises administering a compound of formula (IA):

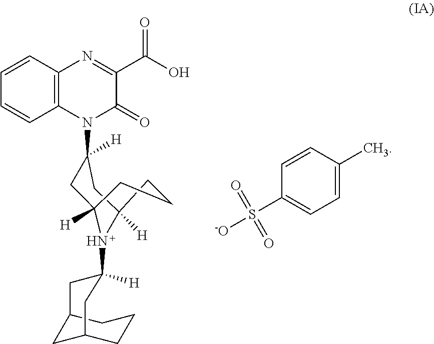

43. The method of any one of claims 40-42, wherein said sleep disorder is an insomnia condition, a hypersomnia condition, a circadian rhythm sleep-wake disorder, an alcohol-induced sleep disorder, insomnia associated with alcohol cessation, or any combination thereof.

44. A method of treating overactive bladder syndrome in a human subject in need thereof, comprising administering to the human subject an effective amount of a compound of formula (I): 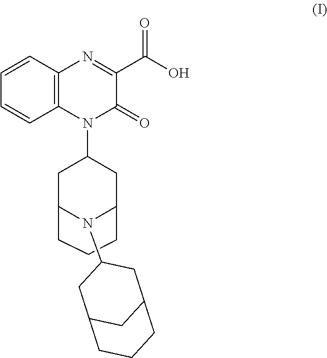 [SMILES: O=C(O)c1nc2ccccc2n(C2CC3CCCC(C2)N3C2CC3CCCC(C3)C2)c1=O]
or a pharmaceutically acceptable salt thereof,
wherein the human subject has a mild or mild to moderate renal impairment.

45. The method of claim 44, wherein the human subject with mild renal impairment has an estimated glomerular filtration rate (eGFR) of about 60 mL/min to about 89 mL/min.

46. The method of claim 44, wherein the human subject with mild to moderate renal impairment has an eGFR of about 45 mL/min to about 59 mL/min.

47. The method of any one of claims 44 to 46, wherein administering the compound of formula (I) to the subject results in at least a mean AUC, Cₘₐₓ, Tₘₐₓ, T_{1/2}, or CL/F that is not statistically different from a corresponding mean AUC, Cₘₐₓ, Tₘₐₓ, T_{1/2}, or CL/F in a similarly situated human subject with no renal insufficiency.

48. The method of any one of claims 44 to 47, wherein administering the compound of formula (I) to the human subject results in at least a mean Ae, Fe, or CL_{R} that is not statistically different from a corresponding mean Ae, Fe, or CL_{R} in a similarly situated human subject with no renal insufficiency.

49. The method of any one of claims 44 to 48, wherein the compound is a compound of formula (I′): 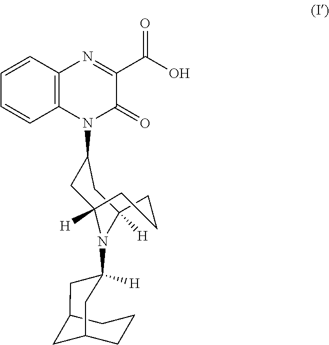
or a pharmaceutically acceptable salt thereof.

50. The method of any one of claims 44 to 49, wherein the method comprises administering a pharmaceutically acceptable salt of the compound of formula (I), wherein the salt is selected from the group consisting of sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, and p-toluenesulfonate salt.

51. The method of any one of claims 44 to 50, wherein the method comprises administering a p-toluenesulfonic acid salt, a sulfate salt, a phosphoric acid salt, or a hydrochloride salt of the compound of formula (I).

52. The method of any one of claims 44 to 51, wherein a p-toluenesulfonic acid salt of the compound of formula (I) is administered.

53. The method of any one of claims 44 to 52, wherein the compound of formula (I) or the pharmaceutically acceptable salt thereof is a compound of formula (IA):

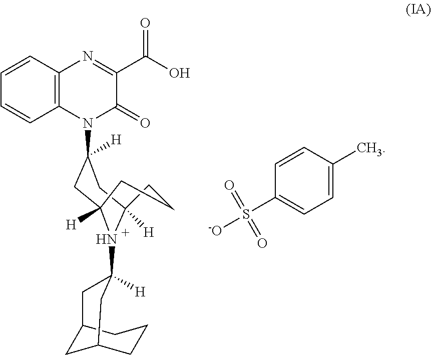

54. The method of claim 53, wherein the compound of formula (IA) is administered orally, parenterally, intravenously, intramuscularly, buccally, or transdermally.

55. The method of claim 53 or 54, wherein the compound of formula (IA) is administered orally.

56. The method of claim 55, wherein the effective amount of the compound of formula (IA) is about 0.10 mg to about 10 mg.

57. The method of any one of claims 53 to 56, wherein the compound of formula (IA) is administered once daily.

58. The method of any one of claims 53 to 57, wherein the compound of formula (IA) is administered at nighttime.

59. The method of any one of claims 53 to 58, wherein the compound of formula (IA) is administered prior to bedtime.

60. The method of any one of claims 53 to 56, wherein the compound of formula (IA) is administered twice daily.

61. The method of claim 60, wherein the compound of formula (IA) is administered approximately every 12 hours.

62. The method of claim 60 or 61, wherein the method comprises administering a first effective amount of the compound of formula (I) or pharmaceutically acceptable salt thereof during daytime, and administering a second effective amount of the compound of formula (I) or pharmaceutically acceptable salt thereof at nighttime prior to bedtime of the human subject.

63. The method of claim 62, wherein the first effective amount is a therapeutically effective amount and is the same as the second effective amount.

64. The method of claim 62, wherein the first effective amount and the second effective amount are different.

65. The method of claim 64, wherein the second effective amount is about 2-fold greater than the first effective amount.

66. The method of claim 64, wherein the second effective amount is about 10-fold greater than the first effective amount.

67. The method of any one of claims 44 to 66, wherein the administration of the compound of formula (I) or a pharmaceutically acceptable salt thereof increases micturition pressure threshold in the human subject by about 30% to 80%.

68. The method of any one of claims 44 to 67, further comprising administering an effective amount of an antimuscarinic agent to the human subject.

69. The method of claim 68, wherein the antimuscarinic agent is oxybutynin, tolterodine, trospium, solifenacin, darifenacin, or a pharmaceutically acceptable salt of any of the foregoing.
